(19) United States
(12) Patent Application Publication
(10) Pub. No.: US 2018/0345006 A1
(43) Pub. Date: Dec. 6, 2018

(54) ELECTRODE APPARATUS

(71) Applicant: CERESTIM LIMITED, London (GB)

(72) Inventor: Alan AMBROSE, London (GB)

(21) Appl. No.: 15/757,780

(22) PCT Filed: Aug. 31, 2016
(86) PCT No.: PCT/GB2016/052688

§ 371 Date: Mar. 6, 2018

(30) Foreign Application Priority Data
Sep. 7, 2015 (GB) 1515831.4

(51) Int. Cl. A61N 1/04; A61B 5/053; A61B 5/00; A61N 1/36; A61B 5/107; G01N 27/02

(52) U.S. Cl. CPC A61N 1/0496; A61B 5/0531; A61B 5/6843; A61N 1/0456; A61B 2018/1472; A61N 1/36031; A61B 5/0536; A61B 5/107; G01N 27/026; A61N 1/0476

(57) ABSTRACT

The invention provides electrode apparatus for non-invasively applying electrical stimulation to a body portion of a human subject by way of a skin interface, the electrode apparatus comprising: an electrode module having: an end for defining an electrolyte application region between the electrode module and the skin interface; and a plurality of electrodes which are electrically couplable or electrically coupled to the skin interface by way of an electrolyte in the said electrolyte application region, the electrodes being spaced apart from each other; and a controller in communication with the electrodes, the controller being configured to individually adjust electrical signals across or between each of the said electrodes and each of one or more pairing electrodes.

### FIELD OF THE INVENTION

The invention relates to electrode apparatus, methods of non-invasively applying electrical stimulation to a body portion of a human subject by way of a skin interface; methods of non-invasively applying electrical stimulation to, or detecting electrical signals from, a body portion of a human subject by way of a skin interface; methods of non-invasively applying a dosage of electrical stimulation to a body portion of a human subject by way of a skin interface; data processing apparatus; methods of estimating a dosage of electrical stimulation impinging on a target treatment region internal to a human body portion; electrode modules for non-invasively applying electrical stimulation to a body portion; and electricity for treatment of a neurological or psychiatric disorder and/or to influence mood and/or cognition.

### BACKGROUND TO THE INVENTION

Neuromodulation is widely used to study and treat the brain (as well as other parts of the body), presenting an attractive alternative for pharmacology treatment. Neuromodulator devices that are on the market include invasive technologies such as deep brain stimulation (DBS), vagus nerve stimulation (VNS), implanted electro-cortical stimulation (IES) and epidural cortical stimulation (ECS). For example, in the United States, the Food and Drug Administration (FDA) has approved DBS for Parkinson's disease, dystonia and obsessive compulsive disorder (OCD) and VNS for epilepsy and depression. Neuromodulators include as well non-invasive technologies such as transcranial magnetic stimulation (TMS), electro-therapy stimulation (CES), transcranial direct current stimulation (tDCS), transcranial alternating current stimulation (tACS), and trigeminal nerve stimulation (TNS). The invasive technologies rank high in spatial resolution but require expensive and risky neurosurgery and are therefore not used in less extreme cases. Therefore there is considerable advantage to be gained if the majority of benefits of the invasive techniques can be achieved but with the lower cost and risk of the non-invasive techniques.

Transcranial Electrical Stimulation (TES), which incorporates tDCS and tACS and other more complex applications, involves the application of small electrical currents to the scalp, generally less than 2 mA, using two or more electrodes. The currents are targeted at particular areas of the brain depending on the result intended or the condition to be treated. For example, for depression, the currents are generally directed at the left dorsolateral prefrontal cortex (DLPFC) (see e.g. Dokos, Socrates, and Colleen K. Loo. “Clinical Pilot Study and Computational Modeling of Bitemporal Transcranial Direct Current Stimulation, and Safety of Repeated Courses of Treatment, in Major Depression”, Journal of ECT, 2015). The currents used may be simple direct currents, simple alternating currents or much more complex signals. A critical component of the TES set-up is the design of the electrodes and the nature of the electrolyte used to bridge the electrode-to-skin gap. The electrodes need to be of sufficient size to keep current density within acceptable ranges—generally held to be less than 100 μA cm⁻² (Liebetanz, David, et al. “Safety limits of cathodal transcranial direct current stimulation in rats.” Clinical Neurophysiology 120.6 (2009): 1161-1167). This implies an electrode area of up to 20 cm² for a stimulation current up to 2 mA. It is known that TES could be used in the treatment of any one or more of: neurological disorders; psychiatric disorders; depression; Parkinson's disease; dystonia; obsessive compulsive disorder; epilepsy; migraine; essential tremor; a sleep disorder; pain; mood disorders; attention deficit disorder; addiction (e.g. alcohol or nicotine addiction); Alzheimer's disease; anxiety; aphasia; autism; auditory disorders; bipolar disorder; cerebral palsy; dysphagia; fibromyalgia; hemiparesis; impairment; injury; multiple sclerosis; obesity; post traumatic stress disorder; schizophrenia; stroke; tinnitus; and Tourette's syndrome. It is also known that TES could be used to influence mood of a subject and/or to improve cognition.

FIG. 1A illustrates a typical TES set-up 1 comprising two saline-soaked sponge electrodes 2, 3 held in place by a head strap 4 on a skin interface of the head 5 of a human subject. Electrical signals are applied to the sponge electrodes via the conductive wires 6, 7 and electrode conductors 8, 9, and to the head by way of saline electrolyte between the electrodes 8, 9 and the skin interface. An alternative embodiment is shown in FIG. 1B where two conductive rubber electrodes 10, 11 are connected to signal wires 6, 7 as before. The electrolyte in this case is electro-paste, and the adhesion of the paste to the skin means that no other fastening method is required. A one-dimensional impedance is typically calculated between the two stimulation electrodes to thereby provide an indication of the impedance between the electrodes. However, in the event of a high impedance value being detected between the electrodes, it is difficult to identify which part of the electrical path between the electrodes is causing the high impedance (e.g. (a) due to the electrolyte, (b) due to the skin-scalp interface, (c) due to the skin, skull, intermediate layers or cortex, (d) due to the direct shunt between the electrodes over the skin). This is because the single impedance value does not separate out the contribution from the various portions of the electrical path. In addition, local changes in the impedance between the electrodes and the skin, which could lead to dangerous concentration of current over a small skin area without significant change in the total impedance, cannot be resolved. In practice, if the impedance value is judged to be too high (say higher, than 10 kΩ) the clinician will perform any combination of (a) removing both electrode assemblies, (b) re-applying electrolyte, and (c) cleaning and lightly abrading the skin. This is done, in a trial and error way, until the impedance falls within limits. Each iteration may take several minutes, and it is difficult to maintain the right level of electrical contact between the electrode and the skin during a stimulation session.

It is also difficult to determine the electrical stimulation dosage impinging on a target treatment region internal to the brain. That is, it is difficult to estimate the exact electrical flux which impinges on the neurons in the target treatment region of the brain. Indeed it is difficult to control the penetration of the applied electrical field to the brain through the skin and other upper layers between the electrode and the brain. Having inaccurate control of dosing implies a risk of accidental over-dosing of stimulation; under-dosing and therefore inadequate stimulation; and simply uncontrolled dosing. This lack of control of effective dose thus presents a significant problem. A particular part of this inexact determination of dose relates to the unknown portion of the applied electrical stimulation which is shunted between the electrodes across the skin and therefore does not penetrate into the brain at all.

Another issue with the TES set-ups shown in FIGS. 1A, 1B is that the only way of identifying discomfort of a patient is by communication with the subject. This means that the clinician must constantly monitor the applied stimulation and manually check whether the subject is experiencing any unwanted side-effects. It also means that side effects may only be addressed once the subject is aware of them.

FIGS. 2A-2C show examples of prior art electroencephalography (EEG) electrodes, which include both wet resistive (FIG. 2A), simple dry capacitive (FIG. 2B) and electrodes which actively amplify detected signals (FIG. 2C). A typical wet resistive EEG electrode consists of a signal wire conductor 20, and an electrode body 22 couplable to a skin interface by an electrolyte, for instance electro-gel, to bridge the gap to the skin. This design of electrode makes largely resistive contact with the skin. An EEG signal is very small, typically 10 to 100 micro volts measured at the scalp, and thus subject to pick-up of electrical noise and other artefacts. The dry capacitive electrode 24 of FIG. 2B can make direct contact with the skin, but the contact is largely capacitive and more subject to noise and artefacts than the wet resistive electrode of FIG. 2A. The actively amplified electrode 26 of FIG. 2C amplifies the signals it detects in order to improve the signal to noise ratio. The objective during EEG acquisition is to achieve the best pick-up of the tiny EEG signals for further signal analysis and processing in the most effective way with the best clarity. EEG signals are used for the purposes of investigation or diagnosis.

As shown in FIG. 3, EEG electrodes are usually used with a head cap 28 made of rubber (or similar flexible material) with holes 30 for the electrodes 22 in pre-determined positions on a standard scale or montage e.g. the 10/20 scale. For wet EEG electrodes (FIG. 2A), electro-gel is generally introduced at the scalp surface with a syringe 32 before the electrode 22 is mounted. Therefore positioning of an entire EEG montage, which may contain as many as 128 electrodes, and establishment and checking of conductivity is very time consuming. In addition, it is time consuming to verify and then maintain the correct impedance between the electrodes and the scalp during the measurement process.

In both existing approaches to TES shown in FIGS. 1A, 1B and the existing approach to EEG shown in FIG. 3, the electrolyte between the electrodes and the skin interface is messy, which leads to inconvenience (and low usability) for the clinician and subject. Electrolytes have different inconvenience factors largely dependent on their viscosity. Saline tends to run down the patients head and neck and electro-gel and electro-paste tend to get caught in the subject's hair. Furthermore, the electrolyte between the electrode and the skin interface is prone to drying out (e.g. low viscosity electrolyte, like saline, may evaporate or flow away from the region between the electrode and the skin interface under gravity), thereby leaving insufficient electrolyte between the electrode and the skin interface. This leads to poor electrical conductivity between electrode and skin interface which in the case of EEG leads to poor signal to noise ratio and, in the case of TES, leads to the risk of increased skin sensitivity and current density applied to the scalp by the electrodes. Current density could increase if (under constant current stimulation and due to the partial drying out of electrolyte between an electrode and the skin interface) the current flowing between the electrode and the skin interface flows through only a partial electrolyte channel between the electrode and the skin interface. The clinician thus needs to be vigilant in monitoring the subject throughout stimulation. Relying on the clinician to be vigilant can be risky as deviations may be missed and the subject exposed to overdoses or unwanted side effects.

These usability challenges have contributed to the stalling of transcranial stimulation technology from making the transition from the research lab to the clinic and onward to treatment in the home. There is therefore a need for solutions to the above mentioned problems.

### SUMMARY OF THE INVENTION

A first aspect of the invention provides electrode apparatus for non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the electrode apparatus comprising: an electrode module having: an (first) end for defining (or configured to define) an electrolyte application region (typically comprising electrolyte in use) between the electrode module and the skin interface; and a plurality of (typically individual) electrodes which are electrically couplable or electrically coupled to the skin interface by way of an electrolyte in the said electrolyte application region, the electrodes being spaced apart from each other; and a controller in (typically electrical) communication with the electrodes (the controller typically comprising one or more computer processors), the controller being configured to individually (typically selectively) adjust (typically alternating current (AC), typically current and/or voltage) electrical signals (e.g. voltage and/or current) across or between each of the said electrodes and each of one or more (e.g. respective) pairing electrodes (e.g. in turn).

It will be understood that typically, in use, electrolyte is provided in the electrolyte application region.

By configuring the controller to individually adjust electrical signals across or between each of the electrodes of the electrode module and each of the said one or more pairing electrodes, the (typically individual) impedance or resistance of one or more localised sub-regions of the electrolyte application region can be determined. This allows the application of electrolyte to be better targeted to the localised sub-regions within the electrolyte application region where it is needed to prevent dry spots from occurring and also to prevent too much electrolyte from being applied (which may leak from the electrolyte application region and cause mess). Additionally or alternatively, the electrical signals applied to selected ones of the electrodes of the electrode module can be adjusted so as to keep the current density at each of one or more (typically each of a plurality of) localised sub-regions of the electrolyte application region within safe limits. This helps to improve safety and comfort of the human subject during a treatment session.

By the controller being configured to individually adjust electrical signals across or between each of the electrodes of the electrode module and a pairing electrode, we do not exclude the possibility that, when the electrical signals across or between each of the electrodes of the electrode module and a pairing electrode are individually adjusted, the electrical signals (e.g. current carried by) across or between one or more of the other electrodes of the electrode module and a pairing electrode are also thereby adjusted indirectly. For example, the controller may individually adjust the electrical current carried between one of the electrodes of the electrode module and a pairing electrode to zero (e.g. by opening a switch in series with the electrode of the electrode module), which may cause each of the other electrodes of the electrode module to carry a greater current to compensate for the fact that the said electrode of the electrode module no longer carries any current (e.g. if the voltages across the electrodes of the electrode module and a or the pairing electrode are the same).

Typically the electrodes of the electrode module are arranged in a two dimensional or three dimensional array. That is, the electrode module typically comprises electrodes spaced from each other in two dimensions and/or electrodes spaced from each other in three dimensions. It may be that, when a three dimensional array of electrodes is provided, that a first plurality of electrodes are spaced from each other only in two dimensions, and that a second plurality of electrodes different from the first plurality are spaced from each other in three dimensions.

Typically the controller is configured to individually (typically selectively) adjust (typically alternating current (AC), typically current and/or potential) electrical signals applied to each of the said electrodes of the electrode module.

Typically the controller is configured to individually (typically selectively) determine (e.g. measure) one or more electrical parameters (e.g. current, potential) at each of the said electrodes of the electrode module.

It may be that the controller is provided in the electrode module. More typically the controller is distributed between a plurality of locations. It may be that at least part of the controller is provided in the electrode module. It may be that at least part of the controller is provided in a second electrode module distinct from the (first) electrode module. It may be that at least part of the controller is provided outside of an electrode module (e.g. in a desktop, laptop or tablet computer or in a portable electronic communications device such as a smartphone). It may be that the controller is implemented in hardware or in software, but more typically the controller is implemented in a combination of hardware and software.

It may be that the controller is configured to adjust any one or more of the amplitude, frequency, waveform shape, spectral content, signal pattern of the (typically alternating current (AC), typically current and/or voltage) electrical signals across or between each of the said electrodes of the (first) electrode module and the said each of one or more pairing electrodes.

Typically the electrodes of the electrode module are fixedly (mechanically) coupled to each other. Typically the positions of the electrodes of the electrode module are fixed relative to each other. Typically the electrodes of the electrode module are mechanically coupled to each other.

Typically the electrode module comprises an electrode housing. Typically the electrodes of the electrode module are mechanically coupled to the electrode housing. It may be that the electrode housing houses some or all of the electrodes of the electrode module. Typically the electrode housing houses at least part of the controller.

Typically the said (first) end of the electrode module comprises a surface for defining the electrolyte application region (typically comprising electrolyte in use) between the electrode module and the skin interface.

Typically the said (first) end of the electrode module comprises the electrodes of the (first) electrode module. It may be that some or all of the electrodes of the electrode module are spaced from the said surface of the said (first) end of the electrode module. It may be that the said surface is substantially planar. Typically some or all of the electrodes of the electrode module are spaced from the said surface of the said (first) end of the electrode module in a direction perpendicular to a or the plane in which the said surface extends. It may be that, in use, one or more or each of the electrodes of the electrode module are closer to the skin interface than the said surface of the said (first) end of the electrode module (or at least the portion of the said surface of the said (first) end to which the said electrodes are mechanically coupled) is to the skin interface.

Typically the said (first) end of the electrode module is an (first) end of the electrode housing. Typically the said surface of the electrode module is a surface of the electrode housing.

It may be that the controller is configured to determine a spatial distribution of current flow within the said electrolyte application region by: individually adjusting electrical signals across or between each of two or more (or each) of the said electrodes of the electrode module and each of one or more (e.g. respective) pairing electrodes (e.g. in turn); determining (e.g. measuring) one or more respective electrical parameters (e.g. current, voltage and/or impedance) which are responsive to the adjusted electrical signals; and determining the spatial distribution of current flow within the said electrolyte application region from the said determined (e.g. measured) electrical parameters.

It may be that the controller is configured to determine the said spatial distribution of current flow within the said electrolyte application region by: determining (e.g. measuring) a parameter indicative of at least the magnitude of the current flowing within (e.g. current density at) each of a plurality of localised sub-regions of the electrolyte application region from the said determined (measured) electrical parameters.

It may be that, for each localised sub-region, the said parameter indicative of at least the magnitude of the current flowing within the localised sub-regions is indicative of a magnitude of the current flowing in that localised sub-region relative to the magnitude of the current flowing in one or more or each of the other localised sub-regions of the said plurality. Alternatively, the said parameter is indicative of an absolute magnitude of the current flowing within the localised sub-region (e.g. in Amperes).

By determining parameters indicative of at least the magnitude of electrical current flowing in (e.g. current density at) each of the said localised sub-regions, the spatial current (e.g. current density) distribution in the electrolyte application region can be readily determined. It can be determined (e.g. by the controller) whether (and where) the localised current flow (e.g. localised current density) at any of the localised sub-regions across the electrolyte application region exceeds a (e.g. safe or recommended) threshold. This allows the application of additional electrolyte to be better targeted to the localised sub-regions within the electrolyte application region where the current flow (e.g. current density) exceeds a predetermined (e.g. safe or recommended) threshold. Additionally or alternatively, the (e.g. amplitude of the) current applied to localised sub-regions having a current flow (e.g. current density) which exceeds the said threshold can be reduced by adjusting the electrical signals applied to individual electrode(s) supplying current to the sub-region. This helps to improve the safety of the apparatus.

It will be understood that, by a localised sub-region, we mean a portion of the electrolyte application region less than the entire electrolyte application region.

Typically one or more or each of the said localised sub-regions comprises the skin interface.

It may be that the estimated current flow (e.g. current density) is different at two or more of the said localised sub-regions.

Typically each said localised sub-region comprises one or more respective said electrodes of the (first) electrode module. Typically the controller is configured to determine the said parameter indicative of the current flowing within each of the said localised sub-regions by: individually adjusting electrical signals across or between at least one electrode of the localised sub-region and each of one or more pairing electrodes; determining (e.g. measuring) one or more electrical parameters (e.g. current, voltage and/or impedance) which are responsive to the adjusted electrical signals; and determining the said parameter indicative of the current flowing within the localised sub-regions from the determined (e.g. measured) electrical parameters.

It may be that the number of localised sub-regions equals the number of electrodes of the electrode module.

It may be that one or more or each of the electrodes are provided on (typically elongate) a respective axial member (typically having a distal end which extends to or beyond or from the said surface of the electrode module).

It may be that the number of localised sub-regions equals the number of axial members of the electrode module, each of the localised sub-regions comprising a said axial member.

Typically the electrode module has a second end opposite the said (first) end. Typically two or more (or the electrodes of one or more pairs) of the electrodes of the (first) electrode module (e.g. a said electrode and a said pairing electrode) are spaced apart from each other in a direction having a component parallel to the line of shortest distance extending between the first and second ends of the (first) electrode module. Typically two or more (or the electrodes of one or more pairs) of the electrodes of the electrode module are spaced from each other in a direction perpendicular to a plane in which the said surface of the electrode module extends. Typically two or more of the electrodes of the (first) electrode module (e.g. a said electrode and a said pairing electrode) are spaced apart from each other in a direction having a component perpendicular to the line of shortest distance extending between the first and second ends of the (first) electrode module. Typically two or more of the electrodes of the (first) electrode module (e.g. a said electrode and a said pairing electrode) are spaced apart from each other across the said (first) end of the electrode module. Typically two or more of the electrodes of the (first) electrode module (e.g. a said electrode and a said pairing electrode) are spaced apart from each other in a direction having a component parallel to the line of shortest distance extending between the first and second ends of the (first) electrode module and having a component perpendicular to the line of shortest distance extending between the first and second ends of the (first) electrode module.

It may be that two or more of the electrodes of the (first) electrode module (e.g. a said electrode and a said pairing electrode) are provided (e.g. mounted) on the same axial member. It may be that two or more of the said electrodes (e.g. a said electrode and a said pairing electrode) are provided (e.g. mounted) co-axially (optionally concentrically) on the same axial member. It may be that the axial members are spaced from each other in a direction having a component perpendicular to the line of shortest distance extending between the first and second ends of the (first) electrode module. It may be that the axial members have longitudinal axes which are parallel to the line of shortest distance extending between the first and second ends of the (first) electrode module.

It may be that one or more or each of the electrodes of the (first) electrode module are annular. It may be that each of one or more or all of the annular electrodes of the (first) electrode module are mounted to a respective axial member by way of an annulus of the annular electrode (e.g. the annulus may receive the axial member). It may be that the electrodes comprise electrical conductors. It may be that the electrodes comprise metal. It may be that the electrodes comprise a conductive elastomer (e.g. elastomer comprising conductive material).

It may be that the controller is configured to selectively adjust electrical signals applied to the said electrodes of the (first) electrode module by way of a multi-channel signal generator.

Typically the controller is configured to selectively measure electrical signals from each of the said electrodes of the (first) electrode module (e.g. by way of a multi-way switch).

It may be that the controller is configured to determine the spatial distribution of electrical current within the electrolyte application region by individually (typically selectively) adjusting electrical signals (e.g. electrical signals predetermined in accordance with a dosage regime) already being applied between one or more of the electrodes of the (first) electrode module and the said one or more pairing electrodes.

It may be that the controller is configured to apply electrical stimulation to the body portion (e.g. in accordance with a predetermined dosage regime) by applying electrical signals to the skin interface by way of electrical signals applied between the said electrodes of the (first) electrode module and the said pairing electrode(s).

It may be that the controller is configured to determine an (typically individual, typically electrical) impedance or (typically electrical) resistance of a localised sub-region of the electrolyte application region by: individually (typically selectively) adjusting (e.g. applying, increasing (e.g. an amplitude of), decreasing (e.g. an amplitude of) or removing) electrical signals (e.g. voltage/current) across or between a said electrode of the (first) electrode module (e.g. an electrode provided in or adjacent to (e.g. partially defining) the said localised sub-region of the electrolyte application region) and each of one or more (typically each of a plurality of) pairing electrodes; determining (e.g. measuring) one or more electrical parameters which are responsive to the adjusted electrical signals; and determining the impedance or resistance of the localised sub-region from the said determined (e.g. measured) electrical parameters.

By determining the impedance or resistance of a localised sub-region of the electrolyte application region, the impedance or resistance between the electrode module and the skin interface can be determined at a greater resolution than from a single impedance or resistance measurement of the electrolyte application region as a whole. This allows the current flowing in (e.g. current density at), and the impedance of, the localised sub-region to be better determined and controlled. This significantly reduces the possibility of the localised current density within the electrolyte application region reaching dangerous levels, thereby improving the safety of the apparatus.

It will be understood that by determining the impedance or resistance of a localised sub-region we include the possibility of determining any parameters (or groups of parameters) indicative of the impedance or resistance of the localised sub-region, such as admittance or conductance.

It may be that the said impedance or resistance of the localised sub-region comprises the impedance or resistance between a said electrode (e.g. a said electrode located in the localised sub-region) and the skin interface or between a said electrode (e.g. a said electrode located in the localised sub-region) and a said pairing electrode.

It may be that the impedance or resistance of the localised sub-region comprises the impedance or resistance between two electrodes of the (first) electrode module. For example, it may be that the one or more pairing electrodes comprises another electrode of the electrode module. In this case, it may be that the impedance or resistance of the localised sub-region comprises the impedance or resistance between the said electrode and the said other electrode of the electrode module.

It may be that the controller is configured to determine (e.g. measure) the said electrical parameters in response to one or more test signals (the test signals being applied by adjusting the said electrical signals across or between the electrode and the said one or more pairing electrodes).

It may be that the controller is configured to determine (e.g. measure) the said electrical parameters in response to each of a plurality of test signals of different frequencies (or a (e.g. single) test signal comprising multiple frequencies) to thereby determine the frequency response of the said impedance of the localised sub-region. For example it may be that the controller is configured to apply a plurality of different electrical signals in turn between the said electrode and each of the said one or more pairing electrodes, each of the said different electrical signals having different frequency content, and to determine (e.g. measure) the said one or more electrical parameters responsive to each of the said different electrical signals to thereby determine a frequency response of the impedance of the localised sub-region. It may be that the controller is configured to determine the presence of one or more materials (e.g. electrolyte, air, hair) in the localised sub-region from the said frequency response. It may be that the controller is configured to output an (e.g. audible, visual or tactile) indication of the said material(s) determined to be present in the localised sub-region.

It may be that the electrode module is a first electrode module. It may be that the electrode apparatus comprises a second electrode module, the second electrode module comprising an (first) end for defining (or configured to define) a second electrolyte application region (typically comprising electrolyte in use) between the second electrode module and the skin interface; and one or more electrodes which are electrically couplable or electrically coupled to the skin interface by way of an electrolyte in the said second electrolyte application region. Typically the second electrode module comprises a plurality of electrodes. Typically the electrodes of the second electrode module are spaced apart from each other. It will be understood that typically, in use, electrolyte is provided in the second electrolyte application region. The second electrode module may have any (or any combination) of the features of the first electrode module discussed herein.

Typically the electrodes of the (first) electrode module are electrically insulated from each other within the (first) electrode module (typically they are brought into electrical communication with each other by electrolyte provided in the electrolyte application region), albeit it may be that the controller is capable of electrically coupling sub-sets of (or all of) the electrodes of the (first) electrode module together so that they can be treated as a single electrode (e.g. by applying the same voltage and/or current to each of the electrodes of the sub-set, or to each of the electrodes of the (first) electrode module). Typically there is no fixed electrical coupling between the said electrodes of the electrode module. Typically the said electrodes of the (first) electrode module are configured so that electrical signals to each of the said electrodes can be adjusted individually (and typically selectively). Typically the said electrodes of the (first) electrode module are configured so that the electrical potential of each of the said electrodes can be adjusted individually (typically selectively, typically independently of the electrical potentials of the other electrodes). Typically the said electrodes of the (first) electrode module are configured so that the electrical current flowing through each of the said electrodes can be adjusted individually (and typically selectively).

It may be that the pairing electrode(s) comprise one or more (or each) of the other electrode(s) of the (first) electrode module and/or one or more (or each of the) electrodes of the second electrode module. The said pairing electrode(s) may comprise (or consist of) a plurality of electrode elements of the second electrode module electrically coupled together such that they can be treated as a single electrode.

It may be that the said one or more pairing electrodes comprises a plurality of pairing electrodes. Typically the said plurality of pairing electrodes comprises one or more electrodes spaced from the said electrode in each of first and second dimensions. Typically, the said plurality of pairing electrodes comprises one or more electrodes spaced from the said electrode in each of first, second and third dimensions.

It may be that the controller is configured to determine the (typically individual, typically electrical) impedance or (typically electrical) resistance of the localised sub-region of the electrolyte application region by individually (typically selectively, typically independently) adjusting (e.g. applying, increasing (e.g. an amplitude of), decreasing (e.g. an amplitude of) or removing) electrical signals across or between the said electrode of the (first) electrode module and each of a plurality of pairing electrodes, typically in turn (e.g. across or between the said electrode and each of a plurality of pairing electrodes of the electrode module and/or each of a plurality of pairing electrodes of the second electrode module in turn). Typically the controller is further configured to: determine (e.g. measure), in each case, one or more respective electrical parameters which are responsive to the adjusted electrical signals; and determine the impedance or resistance of the localised sub-region from the said determined (e.g. measured) electrical parameters.

It may be that the said impedance or resistance is determined by mathematical optimisation of a mathematical model of the impedance of the electrolyte application region (e.g. it may be that a mathematical impedance model is iteratively optimised by reference to the determined electrical parameters, typically until an objective function of the mathematical model meets one or more accuracy criteria). For example, it may be that the controller is configured to: provide an initial impedance model of the electrolyte application region (which may be assume a uniform impedance across the electrolyte application region); individually (typically selectively) apply electrical signals between each of the said electrodes and each of one or more (typically each of two or more) pairing electrodes (e.g. in turn); in each case determining (e.g. measuring) a voltage across and/or a current flowing between each of the said electrodes of the (first) electrode module and the said one or more pairing electrodes; and adjust the impedance model in accordance with (e.g. to better conform to) the said measured voltages across and/or currents flowing between each of the said electrodes of the (first) electrode module and the said one or more pairing electrodes.

It may be that the one or more electrical parameters the controller is configured to determine (e.g. measure) comprise the voltage across and/or the current flowing between the said electrode of the (first) electrode module and the (e.g. respective) pairing electrode(s). In this case, the controller is typically configured to determine the impedance or resistance of the localised sub-region from the voltage and/or current measurements between the said electrode of the (first) electrode module and the (e.g. respective) pairing electrode(s). For example, it may be that the electrical signals are applied by a constant current source, in which case the controller is configured to adjust the electrical signals between the electrode and the pairing electrode by adjusting the voltage across the electrode and the said pairing electrode. In this case, it may be that the controller is configured to measure the voltage across the electrode and the pairing electrode and to determine the impedance or resistance between them from the measured voltage and the (known) constant current output by the constant current source. In some cases, the controller may also be configured to measure the current flowing between the electrode and the pairing electrode, and to determine the impedance or resistance from the measured voltage and current across and between the electrode and pairing electrode.

Alternatively, it may be that the electrical signals are applied by a constant voltage source, in which case the controller is configured to adjust the electrical signals between the electrode and the pairing electrode by adjusting the current flowing between the electrode and the said pairing electrode. In this case, it may be that the controller is configured to measure the current flowing between the electrode and the pairing electrode and to determine the impedance or resistance between them from the known (constant) voltage applied by the voltage source and the measured current flowing between the electrode and the pairing electrode. In some cases, the controller may also be configured to measure the voltage across the electrode and the pairing electrode, and to determine the impedance or resistance from the measured voltage and current across and between the electrode and pairing electrode.

Alternatively it may be that the controller is configured to apply (typically the same, typically AC) electrical signals between each of the said plurality of electrodes of the (first) electrode module and one or more pairing electrodes (typically simultaneously). Typically the controller is configured to determine (e.g. measure) a first (e.g. total) voltage across and/or a first (e.g. total) current flowing between the said electrodes and the one or more pairing electrodes. Typically the controller is configured to then individually (typically selectively) adjust (e.g. remove) electrical signals applied between the said electrode of the (first) electrode module and the one or more pairing electrodes. The controller is typically configured to determine (e.g. measure) a second (e.g. total) voltage across and/or a second (e.g. total) current flowing between the said one or more pairing electrodes and the said electrodes of the (first) electrode module. It may be that the controller is configured to compare the first and second voltage and/or current measurements to determine the impedance or resistance of the localised sub-region of the electrolyte application region. Accordingly, it may be that the one or more parameters determined (e.g. measured) by the controller include a current difference between the first and second currents or a voltage difference between the first and second voltages. In this case, the controller is typically configured to determine the impedance or resistance of the localised sub-region from the said current and/or voltage difference. For example, for a constant current source, it may be that the controller is configured to measure first and second voltages as above, and to determine the impedance or resistance of the localised sub-region by determining the difference between the first and second voltages and dividing the said difference by the (known) constant current output by the constant current source. For a constant voltage source, it may be that the controller is configured to measure first and second currents as above, and to determine the impedance or resistance of the localised sub-region by determining the difference between the first and second currents and dividing the (known) constant voltage output by the constant voltage source by the said difference. It may be that the controller is further configured to compare the determined impedance or resistance with a predetermined threshold impedance or resistance, to thereby determine whether the determined impedance or resistance is acceptable.

It may be that individual electrodes, or sub-sets of electrodes, of the (first) or each electrode module are physically and/or electrically segregated from other individual electrodes or sub-sets of electrodes of that module (e.g. by electrically insulating walls extending between them, which typically form a seal with the skin interface). It may be that the walls are hexagonal in shape (e.g. when viewed in plan along a line of shortest distance between the first and second ends of the electrode module). It may be that the said walls define said localised sub-regions of the electrolyte application region.

Typically the impedance or resistance of the localised sub-region is indicative of the current flowing within the localised sub-region.

It may be that the controller is configured to determine (e.g. a magnitude of) a current flowing within (e.g. the current density of) the said localised sub-region from the said determined impedance or resistance of the localised sub-region.

It may be that the one or more pairing electrodes comprises a plurality of pairing electrodes, each of the said plurality of pairing electrodes being other said electrodes of the (first) electrode module. It may be that the said plurality of pairing electrodes comprises one or more electrodes which neighbour the said electrode (or provided closest to the said electrode) and one or more electrodes remote from the said electrode (e.g. one or more other said electrodes of the electrode module being provided closer to the said electrode than the said remote electrode, or one or more said other electrodes of the electrode module being provided between the said electrode and the said remote electrode). It may be that the controller is configured to determine a current flow (e.g. current density) in each of one or more (preferably in each of two or more) localised sub-regions of the electrolyte application region by comparing impedance or resistance values measured between the said electrode and the said plurality of pairing electrodes.

For the avoidance of doubt, the term “electrode” is used herein to include a single physical electrode element or a plurality of physical electrode elements which are (typically electrically) coupled together such that they can be treated as a single electrode.

It may be that the electrical signals across or between the electrode and each of the one or more pairing electrodes are adjusted in accordance with one or more test signals. In this case, it may be that the controller is configured to determine (e.g. measure) the said one or more electrical parameters while each of the said test signals are being applied (e.g. between the electrode of the electrode module and a pairing electrode), and optionally for a short time thereafter until the effects of the test signal vanish.

It may be that the controller is configured to determine the (typically individual) impedance or resistance of each of a plurality of localised sub-regions of the electrolyte application region by: individually adjusting (e.g. individually applying, increasing (e.g. an amplitude of), decreasing (e.g. an amplitude of) or removing) electrical signals across or between each of the said plurality of the said electrodes of the electrode module and each of one or more respective (typically each of a plurality of respective) pairing electrodes; determining (e.g. measuring) one or more respective electrical parameters which are responsive to the adjusted electrical signals; and determining the impedance or resistance of each of the localised sub-regions from the respective determined (e.g. measured) electrical parameters.

It may be that the impedances or resistances of the localised sub-regions are determined relative to each other. Additionally or alternatively it may be that the impedances or resistances of the localised sub-regions are determined absolutely (e.g. in Ohms).

By determining the impedance or resistance of each of a plurality of localised sub-regions of the electrolyte application region, the impedance or resistance between the electrode module and the skin interface can be determined at a greater resolution than a single impedance of the electrolyte application region as a whole. This allows the current flow through (e.g. current density of) the localised sub-regions to be determined and/or the impedance or resistance of the localised sub-regions to be better controlled. This significantly reduces the possibility of the localised current density within the electrolyte application region reaching dangerous levels, thereby improving the safety of the apparatus.

It may be that the impedance or resistance of each of the said localised sub-regions comprise the impedance or resistance between a said electrode (e.g. an electrode located in the localised sub-region) and the skin interface.

It may be that the impedance or resistance of each of the said localised sub-regions comprise the impedance or resistance between a said electrode (e.g. located in the localised sub-region) and another said electrode of the (first) electrode module. For example, it may be that, in each case, the one or more respective pairing electrodes comprise one or more other electrodes of the electrode module. In this case, it may be that the impedances or resistances of the said localised sub-regions comprise the impedances or resistances between the said electrode and each of the said other electrode(s) of the electrode module.

It may be that the controller is configured to determine the (typically individual) impedance or resistance of each of the said localised sub-regions of the electrolyte application region by individually adjusting electrical signals applied between each of the electrodes of the (first) electrode module and the said each of one or more respective pairing electrodes in turn. It may be that the controller is configured to determine the (typically individual) impedance or resistance of the said localised sub-regions by individually adjusting electrical signals applied between each of one or more electrodes of the (first) electrode module provided in or adjacent to (e.g. partially defining) the said localised sub-region and the said each of one or more respective pairing electrodes (e.g. in turn). Typically the controller is further configured to: determine (e.g. measure), in each case, one or more respective electrical parameters which are responsive to the adjusted electrical signals; and determine the impedance or resistance of the respective localised sub-region from the said determined (e.g. measured) electrical parameters.

It may be that the controller is configured to determine the (typically individual, typically electrical) impedance or (typically electrical) resistance of each of the plurality of localised sub-regions by individually (typically selectively) adjusting (e.g. applying, increasing (e.g. an amplitude of), decreasing (e.g. an amplitude of) or removing) electrical signals across or between each of the said electrodes of the (first) electrode module and a pairing electrode (e.g. across or between each of the said electrodes and another electrode of the (first) electrode module or an electrode of the second electrode module). For example, it may be that the controller is configured to individually adjust a voltage across, and/or a current flowing between, each of the said electrodes of the (first) electrode module and a pairing electrode (e.g. across or between the each of the said electrodes of the (first) electrode module and another electrode of the (first) electrode module or an electrode of the second electrode module, where provided), typically in turn. The pairing electrode may comprise (or consist of) a plurality of electrode elements of the second electrode module electrically coupled together such that they can be treated as a single electrode. It may be that the controller is configured to determine the (typically individual, typically electrical) impedance or (typically electrical) resistance of each of a plurality of localised sub-regions of the electrolyte application region by individually (typically selectively, typically independently) adjusting (e.g. applying, increasing (e.g. an amplitude of), decreasing (e.g. an amplitude of) or removing) electrical signals across or between each of the said electrodes and the same pairing electrode.

It may be that the controller is configured to determine the (typically individual, typically electrical) impedance or (typically electrical) resistance of each of a plurality of localised sub-regions of the electrolyte application region by individually (typically selectively, typically independently) adjusting (e.g. applying, increasing (e.g. an amplitude of), decreasing (e.g. an amplitude of) or removing) electrical signals across or between each of the said electrodes and each of a plurality of respective pairing electrodes (e.g. across or between each of the said electrodes and each of a plurality of the other electrodes of the (first) electrode module and/or each of a plurality of respective pairing electrodes of the second electrode module), typically in turn. Typically, in each case, the said plurality of respective pairing electrodes comprises one or more electrodes spaced from the said electrode in each of first and second dimensions. Typically, the said plurality of respective pairing electrodes comprises one or more electrodes spaced from the said electrode in each of first, second and third dimensions.

It may be that the said impedances or resistances of the localised sub-regions are determined by mathematical optimisation of a mathematical model of the said impedances or resistances (e.g. it may be that a mathematical impedance model is iteratively optimised by reference to the determined electrical parameters, typically until an objective function of the mathematical impedance model meets one or more accuracy criteria).

It may be that the said parameters determined (e.g. measured) by the controller from which the impedances or resistances of the localised sub-regions can be derived comprise the voltage across and/or the current flowing between each of the said electrodes of the (first) electrode module and the respective pairing electrode(s). In this case, the controller is typically configured to determine the impedance or resistance of the localised sub-regions from the voltage and/or current measurements between each of the said electrodes of the (first) electrode module and the respective pairing electrode(s). For example, it may be that the electrical signals are applied by a constant current source, in which case the controller is configured to adjust the electrical signals between each of the said plurality of electrodes and the respective pairing electrode(s) by adjusting the voltage across the said electrode and the said pairing electrode. In this case, it may be that the controller is configured to measure the voltage across the electrode and the pairing electrode and to determine the impedance or resistance between them from the measured voltage and the (known) constant current output by the constant current source. In some cases, the controller may also be configured to measure the current flowing between the electrode and the pairing electrode, and to determine the impedance or resistance from the measured voltage and current across and between the electrode and pairing electrode.

Alternatively, it may be that the electrical signals are applied by a constant voltage source, in which case the controller is configured to adjust the electrical signals between the electrode and the pairing electrode by adjusting the current flowing between the electrode and the said pairing electrode. In this case, it may be that the controller is configured to measure the current flowing between the electrode and the pairing electrode and to determine the impedance or resistance between them from the known (constant) voltage applied by the voltage source and the measured current flowing between the electrode and the pairing electrode. In some cases, the controller may also be configured to measure the voltage across the electrode and the pairing electrode, and to determine the impedance or resistance from the measured voltage and current across and between the electrode and pairing electrode.

Alternatively it may be that the controller is configured to apply (typically the same) electrical signals across or between the electrodes of the (first) electrode module and a pairing electrode (typically simultaneously). Typically the controller is configured to determine (e.g. measure) a first (e.g. total) voltage across and/or a first (e.g. total) current flowing between the said electrodes and the pairing electrode. Typically the controller is configured to then individually (typically selectively) adjust (e.g. remove) electrical signals across or between each of the said electrodes of the (first) electrode module and the pairing electrode in turn. For each adjusted electrical signal, the controller is typically configured to determine (e.g. measure) respective second (e.g. total) voltages across and/or a second (e.g. total) currents flowing between the pairing electrode and the said electrodes of the (first) electrode module. It may be that the controller is configured to compare the first and second voltage and/or current measurements to determine the impedance or resistance of each of the said localised sub-regions of the electrolyte application region. For example, for a constant current source, it may be that the controller is configured to measure first and respective second voltages as above, and to determine the impedance or resistance of a respective localised sub-region by determining the difference between the first and respective second voltages and dividing the said difference by the (known) constant current output by the constant current source. For a constant voltage source, it may be that the controller is configured to measure first and respective second currents as above, and to determine the impedance or resistance of a respective localised sub-region by determining the difference between the first and respective second currents and dividing the (known) constant voltage output by the constant voltage source by the said difference. It may be that the controller is further configured to compare the determined impedances or resistances with a predetermined threshold impedance or resistance, to thereby determine whether each of the determined impedances or resistances are acceptable.

It may be that individual electrodes, or sub-sets of electrodes, of the (first) or each electrode module are physically and/or electrically segregated from other individual electrodes or sub-sets of electrodes of that module (e.g. by electrically insulating walls extending between them, which typically form a seal with the skin interface). It may be that the walls are hexagonal in shape (e.g. when viewed in plan along a line parallel to the line of shortest distance between the first and second ends of the electrode module). It may be that the said walls define said localised sub-regions of the electrolyte application region.

Where the controller is configured to individually adjust electrical signals applied between a said electrode of the (first) electrode module and a pairing electrode by increasing (e.g. an amplitude of) existing electrical signals applied between them, it may be that the (e.g. peak or r.m.s.) amplitude of the increase is less than 50% of the (e.g. peak or r.m.s.) amplitude of the existing electrical (e.g. stimulation) signals applied to that electrode, preferably less than 25%, more preferably less than 10%.

It may be that the controller is configured to determine (e.g. measure) the said electrical parameters in response to respective test signals (the test signals being applied by adjusting the said electrical signals across or between each of the said electrodes and the said one or more respective pairing electrodes, e.g. in turn).

It may be that the controller is configured to determine (e.g. measure) the said electrical parameters in response to each of a plurality of test signals of different frequencies (or a (e.g. single) test signal comprising a plurality of frequencies) to thereby determine the frequency response of the said impedance of the localised sub-region. For example it may be that the controller is configured to apply a plurality of different electrical signals in turn between each of the said electrodes and each of the said one or more respective pairing electrodes, each of the said different electrical signals having different frequency content, and to determine (e.g. measure) the said one or more electrical parameters responsive to each of the said different electrical signals to thereby determine frequency responses of the impedances of each of the localised sub-regions. It may be that the controller is configured to determine the presence of one or more materials (e.g. electrolyte, air, hair) in the said localised sub-regions from the said frequency responses. It may be that the controller is configured to output an (e.g. audible, visual or tactile) indication of the said material(s) determined to be present in the said localised sub-regions.

It may be that the electrical signals across or between each of the electrodes and each of the one or more respective pairing electrodes are adjusted in accordance with one or more test signals. In this case, it may be that the controller is configured to determine (e.g. measure) the said one or more electrical parameters while each of the said test signals are being applied (e.g. between an electrode of the electrode module and a pairing electrode).

Typically the impedances or resistances of the localised sub-regions are indicative of the currents flowing within the said localised sub-regions.

It may be that the controller is configured to determine (e.g. the magnitudes of the) respective current flows within (e.g. the current densities of) each of the said localised sub-regions from the said determined impedances or resistances of the localised sub-regions.

Typically the electrodes of the (first) electrode module are configured such that they are provided in the electrolyte application region in use. It may be that the electrodes of the first electrode module are configured to be in electrical communication with each other by way of the electrolyte in the electrolyte application region.

It may be that, for each said electrode, the one or more respective pairing electrodes comprises a plurality of pairing electrodes, each of the said plurality of pairing electrodes being other said electrodes of the (first) electrode module. It may be that the said plurality of pairing electrodes comprises one or more electrodes which neighbour the said electrode (or provided closest to the said electrode) and one or more electrodes remote from the said electrode (e.g. one or more other said electrodes of the electrode module being provided closer to the said electrode than the said remote electrode, or one or more said other electrodes of the electrode module being provided between the said electrode and the said remote electrode). It may be that the controller is configured to determine a current flow (e.g. current density) in each of one or more (preferably in each of two or more) localised sub-regions of the electrolyte application region by comparing impedance or resistance values measured between the said electrode and the said plurality of pairing electrodes.

It may be that the impedances or resistances of the said localised sub-regions of the electrolyte application region are determined from the said one or more determined electrical parameters or from a change in said one or more determined electrical parameters. It may be that the impedances or resistances are determined from a determined (e.g. measured) current change or a determined (e.g. measured) voltage change.

It may be that the controller is configured to: receive geometry data representing a geometry (e.g. size and/or shape) of the body portion, the body portion comprising a target treatment region internal to the body portion (for example the target treatment region comprising a portion of a human brain); receive impedance data indicative of one or more (typically electrical) impedances or resistances (typically data indicative of impedances of one or more or two or more different types of human tissue such as skin, bone, brain, portions of the brain) of the said body portion; determine electric field data representing an (typically three dimensional) electric field through the body portion, which is responsive to an electrical stimulation applied by electrical signals between one or more of the electrodes of the electrode module and one or more pairing electrodes, taking into account the said geometry data and the impedance data; and determine a dosage of electrical stimulation impinging on the target treatment region from the electric field data.

By taking into account the geometry data and the impedance data in the determination of the electric field applied through the body portion, the dosage of electrical stimulation impinging on the target treatment region (e.g. per unit stimulation applied to the skin interface of the body portion) can be determined more accurately. It can thus be better ensured that a safe dosage of electrical stimulation is impinging on the target treatment region at all times. It can also be determined whether the electrical stimulation impinging on the target treatment region is in accordance with an intended dosage regime.

Typically the geometry data comprises data representative of the size and/or shape of the body portion. Typically the geometry data comprises a mathematical model and/or image of the body portion. For example it may be that the geometry data comprises an image of the body portion obtained by any one of magnetic resonance imaging, computed tomography, electrical impedance tomography or electrical impedance spectroscopy. Alternatively, it may be that the geometry data comprises analytical data defining a simplified geometry of the body portion.

Typically the geometry data represents a three dimensional geometry (e.g. three dimensional size and/or shape) of the human body portion.

For example it may be that the geometry data comprises one or more concentric spheres representing the human head. The geometry data may comprise two or more concentric spheres (e.g. three or four concentric spheres), each sphere representing a different portion of the human head (e.g. brain, skull, scalp).

Alternatively, it may be that the geometry data comprises an image of the body portion obtained by any one or more of magnetic resonance imaging, computed tomography, electrical impedance tomography, electrical impedance spectroscopy.

It may be that the geometry data is specific to a human subject comprising the said body portion. In other cases, it may be that the geometry data is not specific to a human subject.

Typically the geometry data represents a geometry of both an external portion of the body portion and an internal portion of the body portion. For example, the geometry data may represent a geometry of a scalp of a human head and a brain internal to the human head (and typically of the skull and one or more layers between the skull and the brain).

It may be that the geometry data comprises a model or image of the body portion which is not specific to the said human subject. It may be that the geometry data is derived by adjusting an initial model of the body portion (e.g. a model or image of the body portion which is not specific to the said human subject) in accordance with one or more measurements specific to the human subject (e.g. head size and shape, which may be derived from an image of the body portion of the said human subject, such as an image obtained by Magnetic Resonance Imaging and/or a Computed Tomography scan of the target treatment region of the human subject). It may be that the geometry data comprises a model or image of the body portion which is specific to the said human subject.

It may be that the impedance data assumes that the tissue of the body portion is homogeneous (and therefore has the same impedance properties throughout the body portion). More typically, the impedance data comprises impedances of two or more different types of human tissue of the body portion.

It may be that the controller is configured to: apply electrical stimulation to the body portion by adjusting electrical signals applied between the one or more of the electrodes of the electrode module and one or more pairing electrodes; and determine the dosage of electrical stimulation impinging on the target treatment region responsive to the said electrical stimulation applied to the body using the geometry data and the impedance data (e.g. from electric field data derived from the geometry data and the impedance data). It may be that the controller is further configured to adjust the electrical stimulation applied to the body portion by adjusting electrical signals across or between the one or more of the electrodes of the electrode module and the said pairing electrodes responsive to the determined dosage (e.g. to increase or decrease the dosage to better match a predetermined dosage regime or to reduce physiological stress to the human subject).

Typically the controller is configured to determine the electric field data and/or the said dosage of electrical stimulation impinging on the target treatment region taking into account the electrical signals applied across or between the said electrodes and the said pairing electrodes.

Typically the controller is configured to determine the said electric field data taking into account a geometry of the electrode module(s). For example, the controller is configured to determine the said electric field data taking into account a surface area of the electrodes of the electrode modules in contact with the skin interface.

It may be that at least a portion of the geometry data is determined by electrical impedance tomography or electrical impedance spectroscopy of the body portion using the electrodes of the electrode module.

It may be that the impedance data comprises data indicative of an impedance or resistance of a first type of human tissue external to the body portion and data indicative of an impedance or resistance of a second type of human tissue internal to the body portion.

Typically the impedance data comprises data indicative of impedances or resistances of different types of human tissue along an electrical transmission path through the body portion (e.g. between two or more reference positions, the reference positions typically being on an external surface of the body portion, e.g. between one or more of the said electrodes of the electrode module and one or more of the said pairing electrodes).

It may be that the controller is configured to receive impedance data associated with the said geometry data. For example, it may be that the impedance data comprises two or more impedance values, each of which is associated with a different part of the body portion represented by the geometry data.

It may be that the controller is configured to: determine electric field data indicative of an electrical field through the body portion responsive to an electrical stimulation applied to the body portion by the electrodes using the geometry data and the impedance data by: using the said geometry data and the impedance data to mathematically model (e.g. using Maxwell's equations) the electric field applied through the body portion as a function of position responsive to the said electrical stimulation (e.g. per unit stimulation applied to the skin interface of the body portion).

Typically the controller is configured to mathematically model (e.g. using Maxwell's equations) the electric field applied through the body portion as a function of position responsive to the said electrical stimulation using two or more reference positions, each representing a position of an electrode module (or one or more electrodes of an electrode module) on the body portion to and from which the electrical stimulation is provided by way of the skin interface. Typically controller is configured to use the said reference positions in the mathematical modelling process.

It may be that the controller is configured to mathematically model the electric field through the body portion responsive to the said electrical stimulation by mathematically modelling the quasi-static conduction (QSC) approximation to Maxwell's equations. It may be that the controller is configured to mathematically model the electric field through the body portion responsive to the said electrical stimulation by solving the forward problem (i.e. the computation of the electric field distribution in the body portion (e.g. head) resulting from the application of currents to the skin interface (e.g. the scalp)) of the quasi-static conduction (QSC) approximation to Maxwell's equations. It may be that the boundary conditions for the forward problem comprise any one or more (or each) of the following: measured voltages and/or currents at one or more or each of the electrodes; any known voltages and currents determined during measurement of the current shunted across the surface of the skin interface; an assumption that no current flows from the skin into the surrounding air; and an assumption that no current disperses from the head and into the neck.

It may be that the controller is configured to determine a (e.g. instantaneous) dosage of electrical stimulation impinging on the target treatment region responsive to the electrical stimulation by: determining an (typically mathematical, typically three dimensional) impedance model indicative of the impedance or resistance of the body portion as a function of position from the said geometry data and the said impedance data; and using the said impedance model to determine the dosage of electrical stimulation impinging on the target treatment region (e.g. by deriving the electric field data from the impedance model and determining the dosage of stimulation applied to the target treatment region from the electric field data).

It may be that the controller is configured to receive an estimate of an electrical current shunted across the skin interface between the electrode module and a second electrode module, and to determine the dosage of electrical stimulation impinging on the target treatment region from the said determined electric field data taking into account the said estimate of the said electrical current shunted across the skin interface.

It may be that the electric field data is representative of an electric field through each of a plurality of voxels (i.e. discrete volumes) of within the body portion.

Typically the controller is configured to determine a (e.g. instantaneous) dosage of electrical stimulation impinging on the target treatment region by volume integration of the determined electric field through the target treatment region (e.g. the sum of the determined electric field through each of a plurality of voxels representing the target treatment region).

It may be that the controller is further configured to determine a total dosage of electrical stimulation impinging on the target treatment region by time integration of a plurality of said determined instantaneous dosages.

It may be that the controller is configured to determine an (typically mathematical, typically three dimensional) impedance model indicative of the impedance or resistance of the body portion as a function of position by: individually adjusting (e.g. individually applying, increasing (e.g. an amplitude of), decreasing (e.g. an amplitude of) or removing) electrical signals across or between each of the said plurality of the said electrodes of the electrode module and each of one or more respective (typically each of a plurality of respective) pairing electrodes; determining (e.g. measuring) one or more electrical parameters indicative of one or more respective impedances or resistances of the body portion; and determining the impedance model from the said determined (e.g. measured) parameters.

Typically the said one or more pairing electrodes comprise one or more electrodes of a or the second electrode module electrically coupled to the head by a second electrolyte application region between a (first) end of the second electrode module and the skin interface.

Typically the controller is configured to receive geometry data representing a geometry (e.g. size and/or shape) of the body portion (see above) and to determine the said impedance model of the body portion taking into account the said geometry data.

It may be that the impedance model is not specific to the said human subject, but preferably the impedance model is specific to the human subject. It may be that the controller is configured to receive said geometry data specific to the human subject and it may be that the controller is configured to use the geometry data to determine the impedance model.

The controller may be configured to derive from the impedance model a model of voltage and/or electric field and/or current and/or current density through the body portion responsive to an electrical stimulation applied between the said electrodes of the electrode module and the said one or more pairing electrodes.

Typically the controller is configured to determine a (e.g. instantaneous) dosage of electrical stimulation impinging on the target treatment region by volume integration of the determined electric field through the target treatment region (e.g. the sum of the determined electric field through each of a plurality of voxels representing the target treatment region).

It may be that the controller is further configured to determine a total dosage of electrical stimulation impinging on the target treatment region by time integration of a plurality of said determined instantaneous dosages.

Typically the controller is configured to determine the said electric field data taking into account a geometry of the electrode module(s). For example, the controller is configured to determine the said electric field data taking into account a surface area of the electrodes of the electrode modules in contact with the skin interface.

It may be that the controller is configured to: determine electric field data representing an (typically three dimensional) electric field through the body portion, which is responsive to an electrical stimulation applied by electrical signals between one or more of the electrodes of the electrode module and one or more pairing electrodes, taking into account the impedance model; and determine from the electric field data a (e.g. instantaneous) dosage of electrical stimulation impinging on a target treatment region internal to the body portion (for example the target treatment region comprising a portion of a human brain) responsive to an electrical stimulation applied to the skin interface by the said electrodes.

Typically the controller is configured to determine the electric field data and/or the said dosage of electrical stimulation impinging on the target treatment region taking into account the electrical signals applied across or between the said electrodes and the said pairing electrodes.

It may be that the impedance model is further indicative of the impedance or resistance of the electrolyte application region between the (first) electrode module and the skin interface. It may be that the impedance model is further indicative of the impedance or resistance of an electrolyte application region between a second electrode module and the skin interface.

It may be that the impedance model is further indicative of the impedances or resistances of each of a plurality of localised sub-regions of the electrolyte application region between the (first) electrode module and the skin interface. It may be that the impedance model is further indicative of the impedances or resistances of each of a plurality of localised sub-regions of the electrolyte application region between the second electrode module and the skin interface.

It may be that the controller is configured to: provide an initial impedance model; and (e.g. iteratively) adjust (e.g. customise) the initial impedance model by individually (typically selectively) applying electrical signals between each of the said electrodes and each of one or more (typically each of two or more) pairing electrodes (e.g. in turn), in each case measuring a voltage across and/or a current flowing between each of the said electrodes of the (first) electrode module and the said one or more pairing electrodes, and adjusting the impedance model in accordance with (e.g. to better conform to) the said measured voltages across and/or currents flowing between each of the said electrodes of the (first) electrode module and the said one or more pairing electrodes.

It may be that the controller is configured to provide the initial impedance model by: receiving geometry data representing a geometry (e.g. size and/or shape) of the body portion, the body portion comprising a target treatment region internal to the body portion (for example the target treatment region comprising a portion of a human brain); receiving impedance data indicative of one or more (typically electrical) impedances or resistances (typically data indicative of impedances of one or more or two or more different types of human tissue such as skin, bone, brain, portions of the brain) of the said body portion; and deriving the initial (typically three dimensional) impedance model (e.g. an initial impedance model which may not be specific to the said human subject) representing the (typically electrical) impedance or resistance of the body portion as a function of position from the geometry data and the impedance data.

In cases where the impedance model is further indicative of the impedance or resistance of the electrolyte application region(s), the initial impedance model may assume that the impedance or resistance across the electrolyte application region is uniform.

Typically the pairing electrodes comprise one or more electrodes of the second electrode module. This can help to ensure that the electrical signals applied between the electrodes of the (first) electrode module and the pairing electrodes flow through the head (and not just the electrolyte application region).

As mentioned above, it may be that individual electrodes, or sub-sets of electrodes, of the (first) or each electrode module are physically and/or electrically segregated from other individual electrodes or sub-sets of electrodes of that module (e.g. by electrically insulating walls extending between them, which typically form a seal with the skin interface). Again, this helps to ensure that electrical signals applied between the electrodes of the (first) electrode module flow through the head (and not just between electrodes through the electrolyte application region), which helps to better characterise the impedance within the head, thereby allowing more accurate estimate of the dosage of electrical stimulation applied to the target treatment region. It may be that the walls are hexagonal in shape (e.g. when viewed in plan along a line of shortest distance between the first and second ends of the electrode module). It may be that the said walls define said localised sub-regions of the electrolyte application region.

It may be that the controller is configured to adjust the model iteratively (e.g. each time a voltage and/or current measurement is made between an electrode of the (first) electrode module and a pairing electrode following an individual adjustment to the electrical signals across or between the said electrode of the (first) electrode module and the said pairing electrode) in accordance with (e.g. to better conform to) the said measured voltage across and/or a current flowing between each of the said electrodes and the said one or more pairing electrodes. The controller may be configured to adjust the model in accordance with one or more suitable mathematical optimisation techniques (e.g. steepest descent, downhill simplex or simulated annealing). It may be that the controller is configured to perform said one or more mathematical optimisation techniques until one or more accuracy criteria of an objective function are satisfied (e.g. until a least squares error between the measured voltages and currents (or one or more parameters derived therefrom) and the corresponding voltages and currents predicted by the model is less than a predetermined threshold).

It may be that the controller is configured to adjust the impedance model by: individually (typically selectively) applying electrical signals between each of the said electrodes and each of one or more (typically each of two or more) pairing electrodes (e.g. in turn), the electrical signals comprising (AC) electrical signals of different frequencies; determining (e.g. measuring) a frequency response of the impedance to the said electrical signals of a voltage across and/or a current flowing between each of the said electrodes of the (first) electrode module and the said one or more pairing electrodes; and adjusting the impedance model in accordance with (e.g. to better conform to) the said determined (e.g. measured) voltages across and/or currents flowing between each of the said electrodes of the (first) electrode module and the said one or more pairing electrodes.

By applying signals having different frequencies between the said electrodes and the said pairing electrodes, the impedances of different types of human tissue can be identified by virtue of their characteristic frequency responses. Indeed, the controller is typically configured to determine the impedances of different types of human tissue of the body portion from the voltages measured across and/or a current measured flowing between each of the said electrodes and the said one or more pairing electrodes in response to the said signals, and to adjust the model accordingly.

It may be that the impedance model is based on a discrete function, an analytical function or a function defined as points on a finite-element mesh.

Typically the controller is configured to dynamically update the impedance model over time (typically by repeatedly adjusting signals applied between the electrodes of the (first) electrode module and the pairing electrode(s) and making and processing the voltage and/or current measurements as used in the generation of the model).

It may be that the controller is configured to determine a dosage of electrical stimulation impinging on the target treatment region using predetermined data indicative of the position of the target treatment region within the body portion.

For example, the target treatment region may comprise a portion of a human brain internal to a head portion of a human body. In this case, it may be that the predetermined data indicative of the position of the target treatment region within the body portion may comprise data (e.g. a mathematical model or image) indicative of the typical position of the said portion of the human brain within the human brain (e.g. with reference to the geometry data). It may be that the predetermined data is adjusted (e.g. scaled) in accordance with the said geometry data. Thus, it may be that the predetermined data is customised for the human subject.

The target treatment region of the body portion is typically the region of the body portion to which an electrical stimulation dosage regime applied to the electrodes is targeted. For example, the target treatment region of the body portion for the treatment of depression is the dorsolateral prefrontal cortex (DLPFC) of the brain.

It may be that the controller is configured to adjust electrical signals applied to one or more (typically to two or more or each) of the electrodes of the (first and/or second) electrode module(s) (typically to thereby adjust the shape of the electric field applied to the body portion by the electrode module(s)) to thereby adjust (e.g. better focus) the electrical stimulation impinging on the target treatment region, typically responsive to the determined dosage of electrical stimulation impinging on the target treatment region (e.g. to increase or decrease the level of stimulation impinging on the target treatment region in accordance with a dosage regime). For example, the controller may be configured to increase the current carried by the first electrode module and to decrease the current carried by the second electrode module responsive to the determined dosage of electrical stimulation impinging on the target treatment region. Additionally or alternatively, the controller may be configured to change which electrodes of the (first and/or second) electrode module(s) are used to apply stimulation to the body portion by way of the skin interface (e.g. in order to better focus the stimulation on the target treatment region).

It may be that the electrode module is a first electrode module and the electrode apparatus further comprises: a second electrode module having: an (first) end for defining a second electrolyte application region between the second electrode module and the skin interface, the second electrode module comprising one or more electrodes which are electrically couplable or electrically coupled to the skin interface by way of an electrolyte in the said second electrolyte application region; and one or more shunt measurement conductors in (typically electrical) communication with the controller, wherein the controller is configured to: measure one or more electrical parameters (e.g. voltage and/or current and/or impedance or resistance) between one or more electrodes of the first electrode module and one or more of the shunt measurement conductors; and determine a current shunted across the skin interface between the first and second electrode modules taking into account the said one or more measured electrical parameters. It will be understood that typically, in use, electrolyte is provided in the second electrolyte application region.

Typically the controller is configured to (typically selectively, typically individually) adjust electrical signals across or between one or more of the electrodes of the first electrode module and one or more electrodes of the second electrode module.

Typically the controller is configured to determine the current shunted across the skin interface between the first and second electrode modules in response to electrical signals applied between the electrode(s) of the first and second electrode modules taking into account the said one or more measured electrical parameters.

Typically the first electrode module comprises one or more or each of the shunt measurement conductors.

Typically one or more or each of the shunt measurement conductors are provided on or adjacent to the said (first) end of the first electrode module.

Typically one or more or each of the said one or more shunt measurement conductors are configured to be provided in the first electrolyte application region.

Typically one or more or each of the shunt measurement conductors are provided between the electrode(s) of the first electrode module and an edge of the said (first) end of the first electrode module.

Typically the edge of the said (first) end of the first electrode module is provided at or adjacent to the perimeter of the said (first) end of the first electrode module.

Typically the one or more shunt measurement conductors are provided closer to the edge (e.g. the perimeter) of the said (first) end of the first electrode module than the said electrode(s) of the first electrode module are to the said edge.

It may be that one or more or each of the shunt measurement conductors are provided around the one or more electrodes of the first electrode module (or at least projected positions of the electrode(s) onto a plane comprising the said shunt measurement conductors) in a curved, arced, semi-circular or circular arrangement.

It may be that one or more or each of the said shunt measurement conductors substantially surround the electrode(s) of the first electrode module (or at least projected positions of the electrode(s) of the first electrode module onto a plane comprising the said shunt measurement conductors) in two dimensions.

It may be that the controller is configured to: apply one or more electrical (typically AC) test signals (typically an electrical current) between one or more electrodes of the first electrode module and one or more of the shunt measurement conductors; measure one or more electrical parameters across or between (typically a voltage across) the said electrodes of the first electrode module and the said shunt measurement conductors responsive to the said test signal; and determine the said current shunted across the skin interface between the first and second electrode modules taking into account the said one or more measured electrical parameters.

Typically the controller is configured to determine an impedance of the electrical path between the said electrodes of the first electrode module and the said shunt measurement conductors from the said measured electrical parameters, and to determine the said current shunted across the skin interface between the first and second electrode modules taking into account the said determined impedance.

It may be that the test signals are superimposed on electrical stimulation signals (e.g. already being) applied between the electrodes of the first and second electrode modules. The test signals may be applied by, for example, increasing or decreasing (e.g. amplitudes of) electrical stimulation signals (e.g. already being) applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed.

Alternatively, the test signals may be applied in the absence of electrical stimulation signals between the electrodes of the first and second electrode modules.

It may be that the controller is configured to: apply one or more (second) electrical (typically AC, typically current) test signals between the said electrodes of the first electrode module and the said electrodes of the second electrode module; measure one or more electrical parameters (e.g. voltage and/or current) between the said one or more electrodes of the first electrode module and the one or more electrodes of the second electrode module; and determine the said current shunted across the skin interface between the first and second electrode modules further taking into account the said one or more electrical parameters (e.g. voltage and/or current and/or impedance or resistance) measured across or between the said one or more electrodes of the first electrode module and the one or more electrodes of the second electrode module.

It may be that the (second) test signals are superimposed on electrical stimulation signals (e.g. already being) applied between the electrodes of the first and second electrode modules. The second test signals may be applied by, for example, increasing or decreasing (e.g. amplitudes of) electrical stimulation signals (e.g. already being) applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed.

Alternatively, the second test signals may be applied in the absence of electrical stimulation signals between the electrodes of the first and second electrode modules.

It may be that the (first) test signals are applied between the said electrodes of the first electrode module and the said shunt measurement conductors prior to or after the (second) test signals applied across or between one or more of the electrodes of the first electrode module and one or more electrodes of the second electrode module.

Typically the one or more shunt measurement conductors comprises a plurality of shunt measurement conductors spaced apart from each other (typically such that one or more electrical current paths are provided across the skin interface between the said one or more electrodes and the said one or more pairing electrodes which does not pass through any of the one or more shunt measurement conductors).

By spacing the shunt measurement conductors apart from each other, the shunt measurement conductors can be made smaller in size (while still spreading out over a given surface area) to thereby reduce the effect of the shunt measurement conductors on the current shunted along the skin interface from the electrodes.

Typically the plurality of shunt measurement conductors comprises a plurality of shunt measurement conductors spaced apart from each other adjacent to the said edge of the said (first) end of the first electrode module (e.g. a plurality of shunt measurement conductors spaced apart from each other adjacent to the said edge of the said (first) end of the first electrode module).

It may be that each of a plurality of the shunt measurement conductors are spaced equally from the said one or more electrodes of the first electrode module.

It may be that the one or more shunt measurement conductors comprises one or more first shunt measurement conductors and one or more second shunt measurement conductors, the first shunt measurement conductors being positioned closer to the electrodes of the first electrode module than the second shunt measurement conductors are to the electrodes of the first electrode module.

Typically the controller is configured to measure one or more electrical parameters at (one or more or each or all of) the first shunt measurement conductors distinctly from (one or more or each or all of) the second shunt measurement conductors.

Typically the first and second shunt measurement conductors are arranged such that one or more first shunt measurement conductors and one or more second shunt measurement conductors can both detect a current shunted along the skin interface in response to electrical signals applied between electrodes of the first and second electrode modules.

Typically the controller is configured to determine the direction of a current shunted across the skin interface by determining (e.g. measuring) one or more electrical parameters (e.g. current flowing) between the first and second shunt measurement conductors.

It may be that the controller is configured to determine the said current shunted across the skin interface of the said body portion by measuring an electrical parameter across or between (e.g. current flowing between or voltage across) one or more of the first shunt measurement conductors and one or more of the second shunt measurement conductors.

Typically the one or more first shunt measurement conductors and the one or more second shunt measurement conductors are provided in curved, arced, semi-circular or circular arrangements (typically around the electrodes of the first electrode module).

Typically the one or more first shunt measurement conductors comprises a first plurality of shunt measurement conductors and the one or more second shunt measurement conductors comprises a second plurality of shunt measurement conductors.

Typically, within each of the first and second pluralities of shunt measurement conductors, the shunt measurement conductors are spaced apart from each other (typically such that one or more electrical current paths are provided across the skin interface between the first and second electrode modules which do not pass through any of the one or more shunt measurement conductors of the said first and second pluralities).

Typically one or more of the first shunt measurement conductors and one or more of the second shunt measurement conductors are provided on a straight line extending between one or more of the electrodes of the first electrode module and an edge of the said (first) end of the first electrode module (typically along the (first) end of the first electrode module).

It may be that the controller is configured to estimate a dosage of electrical stimulation impinging on a or the target treatment region of (typically internal to) the body portion in response to electrical signals applied between the electrode(s) of the first and second electrode modules taking into account the determined current shunted across the skin interface (between the first and second electrode modules).

The current shunted across the skin interface can be used to more accurately determine a dosage of electrical stimulation impinging on a target treatment region of the body portion (e.g. internal to the body portion). This helps to improve safety, and to ensure that an accurate dosage is applied (e.g. in accordance with a dosage regime) to the target treatment region of the body portion.

It may be that the second electrode module comprises one or more of the said shunt measurement conductor(s).

Typically the one or more shunt measurement conductors are provided on or adjacent to the said (first) end of the second electrode module. Typically the one or more shunt measurement conductors of the second electrode module are provided between the electrode(s) of the second electrode module and an edge of the said (first) end of the second electrode module. Typically the edge of the said (first) end of the second electrode module is provided at or adjacent to the perimeter of the said (first) end of the second electrode module. The shunt measurement conductor(s) of the second electrode module may have any of the features of the shunt measurement conductor(s) of the first electrode module.

Typically the controller is configured to measure one or more electrical parameters (e.g. voltage and/or current and/or impedance or resistance) between one or more electrodes of the second electrode module and the shunt measurement conductors of the second electrode module; and determine the said current shunted across the skin interface between the first and second electrode modules taking into account the said one or more measured electrical parameters between one or more electrodes of the second electrode module and the shunt measurement conductors of the second electrode module.

Typically the controller is configured to: apply one or more electrical (typically AC) (third) test signals (typically an electrical current) between one or more electrodes of the second electrode module and one or more of the shunt measurement conductors of the second electrode module; measure one or more electrical parameters across or between (typically a voltage across) the said electrodes of the second electrode module and the said shunt measurement conductors of the second electrode module responsive to the said (third) test signal; and determine the said current shunted across the skin interface between the first and second electrode modules taking into account the said one or more measured electrical parameters across or between the said electrodes of the second electrode module and the said shunt measurement conductors of the second electrode module.

It may be that the (third) test signals are applied across or between the said electrodes of the second electrode module and the said shunt measurement conductors of the second electrode module prior to or after the (first) test signals applied across or between the said electrodes of the first electrode module and the said shunt measurement conductors and prior to or after the (second) test signals applied across or between one or more of the electrodes of the first electrode module and one or more electrodes of the second electrode module.

Typically the controller is configured to determine an impedance of the electrical path between the said electrode(s) of the second electrode module and the said shunt measurement conductor(s) of the second electrode module from the said measured electrical parameter(s), and to determine a current shunted across the skin interface between the first and second electrode modules taking into account the said determined impedance.

It may be that the (third) test signals are superimposed on electrical stimulation signals applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed. The test signals may be applied by, for example, increasing or decreasing (e.g. amplitudes of) electrical stimulation signals applied between the electrodes of the first and second electrode modules.

Alternatively, the (third) test signals may be applied in the absence of electrical stimulation signals between the electrodes of the first and second electrode modules.

It may be that the controller is configured to measure electrical signals (e.g. voltage, current) between one or more shunt measurement conductors of the first electrode module and one or more shunt measurement conductors of the second electrode module.

It may be that the first and second electrode modules each comprise one or more shunt measurement conductor(s), and the controller is configured to determine the said current shunted across the skin interface of the said body portion taking into account one or more electrical parameters (e.g. voltage and/or current and/or impedance or resistance) measured across or between one or more shunt measurement conductors of the first electrode module and one or more shunt measurement conductors of the second electrode module.

It may be that the controller is configured to determine multiple values for the current shunted across the skin interface between the said one or more electrodes of the first and second electrode modules. It may be that the controller is configured to determine an average (e.g. mean) value from the said multiple values. It may be that the controller is configured to discard outlier values prior to any averaging (that is, it may be that the controller is configured to not include outlier values in the average value).

It may be that the controller is configured to adjust electrical signals applied to one or more electrodes of one or both of the first and second electrode modules (typically to thereby adjust the shape the electric field impinging on the target treatment region of the body portion by the electrodes, typically responsive to the said determined current shunted across the skin interface between the said one or more electrodes of the first and second electrode modules exceeding a threshold) to thereby reduce the current shunted across the skin interface between the first and second electrode modules.

This helps to provide a more targeted dosage of electrical stimulation to the target treatment region of the body portion, and helps to reduce irritation to the skin interface.

It may be that the controller is configured to selectively dispense electrolyte into (typically from one or more electrolyte reservoirs, typically by way of one or more electrolyte delivery lines extending between the said reservoir(s) and) the said electrolyte application region, and/or to selectively remove electrolyte from, the electrolyte application region.

By selectively dispensing electrolyte from the electrolyte reservoir(s) to, and/or selectively removing electrolyte from, the electrolyte application region, it can be ensured that the quantity of electrolyte in the electrolyte application region is correctly controlled. Typically the one or more electrolyte reservoirs are provided in or on the electrode module. By providing the reservoir(s) in the electrode module, a more compact and user friendly arrangement can be provided which makes the electrode apparatus more suitable for use outside of a laboratory or hospital environment (e.g. at the home of the human subject).

It may be that the controller is configured to employ a closed-loop control system to control the selective dispensation the electrolyte reservoir(s) to and/or removal of electrolyte from the electrolyte application region.

By providing a closed-loop control system to control the selective dispensation of electrolyte from the electrolyte reservoir(s) to, and/or removal of electrolyte from, the electrolyte application region, the correct quantity of electrolyte can be provided to the electrolyte application region, thereby reducing or preventing dry-spots within the electrolyte application region and preventing leakage of excess electrolyte from the electrolyte application region. This provides a more convenient apparatus for and reduces mess during the application of electrical stimulation to the human subject.

It may be that the controller (typically a or the closed loop control system) is provided with feedback, the controller being configured to selectively dispense electrolyte to, and/or remove electrolyte from, the electrolyte application region responsive to the said feedback.

It may be that the feedback is indicative of a distribution of electrolyte in the electrolyte application region.

It may be that the feedback is indicative of a degree of contact between the electrolyte and the electrode(s) of the electrode module.

It may be that the feedback is indicative of a quantity of electrolyte in the electrolyte application region or at one or more (typically two or more) localised sub-regions of the electrolyte application region.

It may be that the feedback is indicative of current flow through (e.g. current density in) each of one or more (typically each of two or more) localised sub-regions of the electrolyte application region. It may be that the feedback is indicative of the spatial current distribution within the electrolyte application region.

It may be that the feedback is indicative of impedances or resistances of one or more (typically two or more) localised sub-regions of the electrolyte application region. The feedback may comprise an estimated quantity of electrolyte to be added to and/or removed from the electrolyte application region, or to or from each of a plurality of localised sub-regions of the electrolyte application region. The said estimate may be derived from impedances or resistances of one or more localised sub-regions of the electrolyte application region. The said estimate(s) may be derived from an (e.g. computer generated, typically mathematical, typically dynamically updated) impedance model of the impedance or resistance of each of the localised sub-regions (e.g. as a function of position).

It may be that the controller is configured to dispense electrolyte from the electrolyte reservoir(s) to the electrolyte application region responsive to a determination from the said feedback that an impedance or resistance or current density of the electrolyte application region is outside of an acceptable range.

It may be that the controller is configured to dispense electrolyte from the electrolyte reservoir(s) to, and/or remove electrolyte from, the electrolyte application region by way of one or more (typically two or more) electrolyte ducts provided at, and/or extending through, the said end of the (first) electrode module.

It may be that the controller is configured to dispense electrolyte to, and/or to remove electrolyte from, the electrolyte application region by way of a plurality of electrolyte ducts which are spaced apart from each other across the said (first) end of the electrode apparatus (typically in a direction having a component perpendicular to the line of shortest distance between the said end and a or the second end of the (first) electrode module opposite the said (first) end).

It may be that the plurality of ducts are arranged in a concentric arrangement or in a spiral shape on the said end of the electrode module.

Typically each of a plurality of the electrodes of the (first) electrode module is provided adjacent to a different electrolyte duct.

It may be that the controller is configured to selectively dispense electrolyte from the electrolyte reservoir(s) to, and/or to selectively remove electrolyte from, the electrolyte application region through each of the said electrolyte ducts individually.

Typically the controller is configured to selectively dispense electrolyte from the electrolyte reservoir(s) to, and/or selectively remove electrolyte from, each of a plurality of localised sub-regions within the electrolyte application region individually.

Typically the controller is configured to selectively dispense electrolyte from the reservoir(s) to, and/or selectively remove electrolyte from, a localised sub-region of the electrolyte application region (typically through one or more of the said electrolyte ducts) responsive to feedback specific to that sub-region, e.g. responsive to the feedback providing an indication that there is insufficient or too much electrolyte in that sub-region respectively (typically whilst not dispensing electrolyte into, and/or removing electrolyte from, one or more other localised sub-regions of the electrolyte application region, such as localised sub-regions of the electrolyte application region which are determined to have the correct quantity of electrolyte).

The said feedback specific to the said sub-region may comprise an impedance or resistance between the skin interface and an electrode provided in (or adjacent to) the localised sub-region or an impedance or resistance between two electrodes provided in the localised sub-region.

Typically the electrolyte ducts are configured to be in fluid communication with the electrolyte application region in use.

Typically the controller is configured to individually dispense electrolyte from the electrolyte reservoir(s) to, and/or individually remove electrolyte from, the electrolyte application region through each of the said two or more electrolyte ducts individually by opening an electronically controllable valve associated with that duct. It may be that the controller is configured to individually restrict electrolyte from flowing from the electrolyte reservoir(s) to the electrolyte application region through each of the said two or more electrolyte ducts individually by closing the said electronically controllable valve associated with that duct. Typically the said electronically controllable valves are operable to allow electrolyte to flow from the reservoir(s) to the electrolyte application region through the ducts with which they are associated (or vice versa) when they are in the open position, and to restrict electrolyte from flowing from the reservoir(s) to the electrolyte application region through the ducts with which they are associated when they are in the closed position.

It may be that the controller is configured to selectively dispense electrolyte into, and/or selectively remove electrolyte from, the electrolyte application region by way of one or more electrolyte ducts, each of the electrolyte ducts being provided by (e.g. extending through) a respective axial member extending to or through the said end of the electrode module.

Typically the said axial members are the axial members to which electrodes may be mounted (see above).

It may be that the controller is configured to selectively dispense electrolyte into, and/or selectively remove electrolyte from, each of a plurality of localised sub-regions of the electrolyte application region individually by way of an electrolyte duct of a said axial member provided in or adjacent to the said localised sub-region.

It may be that the apparatus further comprises one or more electrolyte flow directors in communication with the controller, the controller being configured to selectively dispense electrolyte to, and/or selectively remove electrolyte from, the electrolyte application region by activating one or more of the electrolyte flow directors or a respective electrolyte flow director.

Typically the controller is configured to selectively and individually dispense electrolyte from each of the plurality of electrolyte ducts by activating one or more of the electrolyte flow directors or a respective electrolyte flow director.

Each of the electrolyte flow directors may comprise a (e.g. air) pressure gradient generator configurable to selectively provide a (positive (to dispense electrolyte) or negative (to remove electrolyte)) pressure gradient between one or more of the reservoir(s) and one or more of the said electrolyte ducts. Typically the pressure gradient generator comprises a pump. However, it will be understood that any suitable alternative pressure gradient generator could be employed. For example the pressure gradient generator may comprise any one or more of: a piezo-electric pump; a pressurised gas reservoir configurable to selectively apply to the required pressure gradient; off-gassing from a chemical reaction; or gaseous expansion caused by heating. Alternatively, one or more of the electrolyte flow directors may comprise a selective gravity feed, which can be activated by way of one or more mechanical switches or one or more of the said electronically controlled valves.

It may be that the pressure gradient generator is operable to provide positive and negative pressure gradients in different operating modes (e.g. positive gradient in first mode, negative gradient in second mode). It may be that the controller is configured to control whether the pressure gradient generator provides a positive or negative pressure gradient (e.g. by controlling a mode of operation of the pressure gradient generator). It may be that the controller is configured to remove electrolyte from the electrolyte application region by way of one or more electrolyte ducts in communication with the electrolyte application region by applying a negative pressure gradient to the duct by way of the pressure gradient generator. It may be that the controller is configured to remove electrolyte from the electrolyte application region by way of each of a plurality of electrolyte ducts individually by applying a negative pressure gradient to the duct by way of the pressure gradient generator and by opening the electronically controlled valve associated with that duct. It may be that the pressure gradient generator is configured to direct the electrolyte removed from the electrolyte application region to one or more (e.g. one or more of the said) electrolyte reservoirs (for later use).

It may be that the controller is configured to dispense electrolyte to the electrolyte application region by way of one or more electrolyte ducts in communication with the electrolyte application region by applying a positive pressure gradient to the ducts by way of the pressure gradient generator. It may be that the controller is configured to dispense electrolyte from the electrolyte application region by way of each of a plurality of electrolyte ducts individually by applying a positive pressure gradient to the ducts by way of the pressure gradient generator and by opening the electronically controlled valve associated with that duct.

Typically the electrode module comprises the electrolyte flow director(s).

It may be that the one or more electrolyte reservoirs are re-fillable.

Additionally or alternatively the one or more electrolyte reservoirs may be replaceable.

It may be that the one or more electrolyte reservoirs are disposable.

It may be that the controller is configured to adjust (e.g. reduce an amplitude of) electrical signals applied across or between one or more selected electrodes of the (first) electrode module and one or more pairing electrodes responsive to a determination that the impedance or resistance or a current density between one or more of the said electrodes of the (first) electrode module and the skin interface exceeds a predetermined (e.g. safety) threshold.

This helps to improve the safety of the apparatus for application of electrical stimulation to the human subject.

It may be that the apparatus further comprises electrolyte containment apparatus for restricting leakage of electrolyte from the electrolyte application region.

This helps to reduce mess during application of electrical stimulation to the human subject, which in turn improves convenience for the human subject.

It may be that the electrolyte containment apparatus comprises an electrolyte absorber provided on the said (first) end of the electrode module.

Typically the electrolyte absorber at least partially (preferably fully) surrounds at least some of (preferably all of) the electrodes of the electrode module.

Typically the electrolyte absorber is provided around at least part of the (preferably the entire) perimeter of the said (first) end of the electrode module.

It may be that the electrolyte containment apparatus comprises a seal provided on the said (first) end of the electrode module for restricting leakage of electrolyte from the electrolyte application region.

Typically the seal extends around at least part of (preferably the entire) perimeter of the said (first) end of the electrode module.

It may be that the electrolyte containment apparatus comprises a pressure gradient generator in communication with the said (first) end of the electrode module for restricting leakage of electrolyte from the electrolyte application region.

It may be that the pressure gradient generator is configured or configurable to apply a negative pressure gradient between an internal portion of the electrode module (e.g. an electrolyte reservoir provided in the electrode module) and the said (first) end of the electrode module so as to restrict leakage of electrolyte from the electrolyte application region.

It may be that the pressure gradient generator comprises a (e.g. mechanical) pump. It may be that the pressure gradient generator comprises a vacuum pump. It may be that the pressure gradient generator employs any one of: a static pressure reservoir; gas absorption from a chemical reaction; gaseous contraction caused by cooling; or a capillary feed.

Preferably the pressure gradient generator is configured to direct electrolyte from the electrolyte application region to one or more electrolyte reservoirs (typically provided in the electrode module) for later re-use.

It may be that the controller is provided in communication with the pressure gradient generator and is configured to use the pressure gradient generator to selectively apply a (e.g. negative) pressure gradient to restrict leakage of electrolyte from the electrolyte application region. It may be that the electrode apparatus comprises a user control for manually selectively applying a (e.g. negative) pressure gradient to restrict leakage of electrolyte from the electrolyte application region.

It may be that the electrolyte containment apparatus comprises a porous seal provided on the said (first) end of the electrode module for restricting leakage of electrolyte from the electrolyte application region and a pressure gradient generator in communication with the said seal, the pressure gradient generator configured or configurable to apply a (typically negative) pressure gradient between one or more holes in the porous seal and an or the electrolyte reservoir to thereby restrict leakage of electrolyte from the electrolyte application region.

It may be that the electrolyte containment apparatus comprises a plurality of walls provided at (typically extending from the surface of) the (first) end of the electrode module, the said walls defining localised sub-regions within the electrolyte application region and being configured to restrict electrolyte (and typically current) leakage from (or between) the said localised sub-regions when the said (first) end of the electrode module is installed on the skin interface. Typically each of the localised sub-regions comprises one or more electrodes of the (first) electrode module. Typically each of the localised sub-regions comprises one or more (e.g. a single) axial member on which one or more electrodes are mounted. Typically each of the localised sub-regions comprises one or more (e.g. a single) electrolyte duct through which electrolyte can be dispensed into the localised sub-region. By restricting electrolyte and current leakage from localised sub-regions within the electrolyte application region, the current density in each of the localised sub-regions can be more easily controlled.

It may be that the apparatus further comprises one or more sensors configured to measure one or more physiological stress indicators indicative of a physiological stress of the human subject (typically the said physiological stress being responsive to, and/or caused by, the electrical stimulation applied to the subject by way of the electrodes), wherein the controller is configured to: receive the said one or more measured stress indicators from the said sensors; determine whether one or more physiological stress criteria are met taking into account the measured physiological stress indicators; and provide an output responsive to a determination that the said physiological stress criteria are met.

By (typically automatically) detecting one or more physiological stress indicators indicative of a physiological stress of the human subject, action can be taken to reduce or prevent any discomfort experienced by the human subject during an application of electrical stimulation. While this is suitable for a controlled application environment such as a laboratory or hospital, it is particularly suitable for use of the electrode apparatus away from medical supervision, such as in the home of the subject.

It may be that one or more or each of the sensors are provided in or on (e.g. the said end of) the electrode module.

It may be that one or more or each of the sensors are couplable or coupled to the body portion.

It may be that one or more or each of the sensors are couplable or coupled to one or more second body portions of the human subject different from the said body portion.

It may be that one or more or each of the sensors are comprised in a hand-held or wearable electronic device of the human subject (e.g. a personal, typically portable, electronic communications device of the human subject).

It may be that the one or more sensors comprise one or more or each of the electrodes of the (first) electrode module. For example, the controller may be configured to use one or more electrodes of the (first) electrode module in an electroencephalography (EEG) mode in order to measure one or more physiological stress indicators indicative of a physiological stress of the human subject. EEG can be used, for example, to detect the onset of a migraine in the human subject (e.g. by detecting an aura).

It may be that the electrode apparatus further comprises an input device (e.g. a personal, typically portable, electronic communications device of the human subject) by which the human subject can manually enter one or more physiological stress indicators (which are typically taken into account by the controller when determining whether the said physiological stress criteria are met).

It may be that the electrode apparatus comprises a plurality of sensors spaced from each other at the said end of the electrode module (typically in a direction having a component perpendicular to a line of shortest distance between the said end of the electrode module and a second end of the electrode module opposite the said (first) end), each of the sensors being configured to measure (the same or different) physiological stress indicators of the subject.

It may be that the electrode apparatus comprises a plurality of sensors, each of which is configured to measure a said physiological stress indicator at a different localised sub-region of the electrolyte application region.

It may be that the controller is configured to determine a value of a function taking into account the measured physiological stress indicator(s). It may be that the controller is configured to determine that the physiological stress criteria are met if the determined value of the function is outside of an acceptable range (e.g. beyond a limit).

It may be that the controller is configured to determine whether each of the measured physiological stress indicators meets one or more respective physiological stress criteria and to determine that the physiological stress criteria are met responsive to a determination that one or more (or two or more or each) of the measured physiological stress indicators meet the said respective physiological stress criteria.

It may be that the electrode apparatus comprises first and second sensors, the first sensor being configured to measure a first said physiological stress indicator of the human subject and the second sensor being configured to measure a second said physiological stress indicator of the human subject different from the first physiological stress indicator.

It may be that the first said physiological stress indicator is an indicator of a first physiological stress of the subject and the second said physiological stress indicator is an indicator of a second physiological stress of the subject different from the first physiological stress.

It may be that the output provided responsive to the determination that the said physiological stress criteria are met comprises one or more signals which cause a visual, audible and/or tactile notification (e.g. a notification that the said physiological stress criteria are met), such as a warning or an alarm.

It may be that the output provided responsive to the determination that the said physiological stress criteria are met comprises one or more signals for reducing the physiological stress of the human subject.

It may be that the output provided responsive to the determination that the said physiological stress criteria are met comprises one or more signals which adjust the electrical stimulation applied to the body portion by way of the said electrodes.

Typically the electrical stimulation applied to the body portion is adjusted by reducing the amplitude of the electrical signals applied to one or more of the electrodes.

It may be that the controller is configured to adjust the electrical stimulation applied to the body portion by adjusting electrical signals applied to each of two or more electrodes.

For example, it may be that the controller is configured to increase a current carried by a first electrode of the electrode module and to decrease a current carried by a second electrode of the electrode module or vice versa.

In another example (where first and second electrode modules are provided), the controller is configured to increase a current carried by the electrodes of the first electrode module (as a whole) and to decrease a current carried by the electrodes of the second electrode module (as a whole) or vice versa.

It may be that the output provided responsive to the determination that the said physiological stress criteria are met comprises one or more signals which adjust a current distribution between electrodes of the electrode module, or adjust a current distribution between the (first) electrode module and the second electrode module.

It may be that the controller is configured to adjust the electrical stimulation applied to the body portion by adjusting any one or more of the following aspects of the electrical signals applied to one or more of the electrodes: the waveform; frequency content; and polarisation (e.g. by applying a DC offset).

It may be that the output provided responsive to the determination that the said physiological stress criteria are met comprises a signal which causes electrical stimulation being applied to the body portion to be aborted (i.e. turned off).

It may be that the output provided responsive to the determination that the said physiological stress criteria are met comprises a signal which causes electrolyte to be selectively dispensed to the electrolyte application region.

It may be that the output provided responsive to a determination that one or more first physiological stress criteria are met comprises a signal which causes electrolyte to be selectively dispensed to the electrolyte application region or a notification to be provided and the output provided responsive to a determination that one or more second physiological stress criteria different from the first physiological stress criteria are met comprises a signal which causes the electrical stimulation applied to the body portion by the electrodes to be adjusted (e.g. reduced) or aborted.

It may be that each of one or more (typically each of two or more) of the said sensors are configured to measure a physiological stress indicator specific to a respective localised sub-region of the electrolyte application region, wherein the controller is configured to determine whether one or more localised physiological stress criteria are met taking into account the measured physiological stress indicator and to provide an output specific to the said localised sub-region responsive to a determination that said one or more localised physiological stress criteria specific to that sub-region are met.

For example it may be that the controller is configured to provide an output which reduces a physiological stress (e.g. skin sensitivity) specific to a said localised sub-region by individually (and typically selectively) dispensing electrolyte, or reducing electrical stimulation applied by one or more of the electrodes (e.g. by individually adjusting electrical signals applied to one or more electrodes), responsive to a determination that one or more of the said localised physiological stress criteria are met.

It may be that the said one or more sensors comprise one or more sensors configured to measure a physiological stress indicator which comprises a physiological parameter of the body portion (e.g. on the skin interface).

It may be that the said one or more sensors comprise a pH sensor configured to measure a pH of the skin interface.

It may be that the said one or more sensors comprise a temperature sensor configured to measure a temperature of the skin interface.

It may be that the one or more sensors comprise one or more sensors configured to measure one or more physiological stress indicators indicative of a pre-ictal state of the subject (e.g. precursors to fit, migraine or skin lesions).

For example, the one or more sensors may comprise one or more blood pressure sensors.

Additionally or alternatively, the one or more sensors may comprise one or more heart rate monitors configured to determine a heart rate (or changes in the heart rate) of the human subject.

Additionally or alternatively, the one or more sensors may comprise one or more sensors of blood oxygen saturation (such as a pulse oximeter). It may be that the blood oxygen saturation sensor is configured to determine changes in blood oxygen saturation levels (e.g. changes indicative of a pre-ictal state of the subject (e.g. that the subject is about to experience a fit)).

By detecting one or more physiological stress indicators indicative of a pre-ictal state of the human subject, corrective action can be taken before the human subject experiences discomfort.

It may be that the one or more sensors comprise one or more or each of the electrodes of the electrode module configured to operate in an electroencephalography (EEG) mode.

For example, the controller may be configured to use one or more electrodes of the electrode module in an electroencephalography (EEG) mode in order to measure one or more physiological stress indicators indicative of the onset of a migraine in the human subject (e.g. by detecting an aura).

Detection of a migraine aura from the electrodes in EEG mode (see above) may also be considered to be detection of a pre-ictal state of the subject.

It may be that the one or more sensors comprise one or more sensors configured to measure one or more physiological stress indicators indicative of a skin sensitivity of the human subject.

It may be that the said one or more sensors comprise one or more colourimeters configured to measure a parameter indicative of a colour of the body portion (e.g. of the skin interface).

For example the said one or more sensors may comprise one or more light sources (e.g. laser or LED) and one or more light detectors (e.g. photodiode, phototransistor, image detector such as a camera or infrared camera) configured to detect light of a wavelength emitted by the light source. Typically the light source is configured to emit light towards the skin interface. Typically the light detector is configured to detect light emitted by the light source which has been reflected from the skin interface.

It may be that the said one or more colourimeters are configured to measure a parameter indicative of a red or infrared colour of the body portion (e.g. of the skin interface).

The said light source(s) may comprise a light source configured to emit light having a wavelength in the region 620 nm to 750 nm (red light), or in the infrared region. This allows the colourimeter to measure a parameter indicative of the redness of the skin interface, which is a useful (and typically reliable) indicator of the physiological stress of the subject.

It may be that skin redness is a pre-cursor to skin lesions forming. Accordingly, it may be that the colourimeter is a sensor configured to measure a physiological stress indicator (e.g. redness of the skin) indicative of a pre-ictal state of the human subject (e.g. redness of the skin may be a pre-cursor to skin lesions forming).

It may be that the one or more sensors comprise one or more movement sensors.

For example, it may be that the said one or more movement sensors comprise any one or more of the following: accelerometer; gyroscope; magnetometer. By detecting movements which are indicative of a physiological stress (e.g. slumping, shaking, seizure, having a fit) of the subject, it can be determined whether the subject is experiencing a said physiological stress.

The one or more movement sensors may comprise one or more sensors for indirectly detecting movements which are indicative of a physiological stress (e.g. slumping, shaking, seizure, having a fit) of the subject. For example, the movement sensors may comprise any one or more of: heart rate monitor; heart rate variability oximeter; blood pressure detector; temperature sensor; and an electroencephalogram (EEG).

Additionally or alternatively, the one or more sensors may comprise one or more movement sensors (e.g. accelerometer, gyroscope) configured to detect movements indicative of a pre-ictal state of the human subject (e.g. pre-epileptic fit).

It may be that the said one or more sensors are in (e.g. wired or more preferably wireless) data communication with the controller.

It may be that the controller is configured to display the detected level of comfort of the subject visually, either for a medical professional or for the subject themselves (or both).

A second aspect of the invention provides a method of non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the method comprising: providing an electrode module having an (first) end and a plurality of electrodes, the electrodes being spaced apart from each other; defining an electrolyte application region (typically comprising electrolyte in use) between the electrode module and the skin interface using the said end of the electrode module; electrically coupling the said electrodes to the skin interface by providing electrolyte in the said electrolyte application region; and individually (typically selectively) adjusting (typically alternating current (AC), typically current and/or voltage) electrical signals across or between each of the said electrodes and each of one or more pairing electrodes.

A third aspect of the invention provides a method of non-invasively applying (or configured to non-invasively apply) a dosage of electrical stimulation to a body portion (typically to a target treatment region of a body portion internal to the body portion, such as a brain or a portion of the brain) of a human subject by way of a skin interface (e.g. a skin interface of the subject's scalp), the method comprising: providing an electrode module having an (first) end and a plurality of electrodes, the electrodes being spaced apart from each other; defining an electrolyte application region (typically comprising electrolyte in use) between the electrode module and the skin interface using the said end of the electrode module; electrically coupling the said electrodes to the skin interface by providing electrolyte in the said electrolyte application region; applying a dosage of electrical stimulation to the body portion by applying electrical signals to each of the said electrodes; and individually (typically selectively) adjusting (typically alternating current (AC), typically current and/or voltage) electrical signals across or between each of the said electrodes and each of one or more pairing electrodes.

A fourth aspect of the invention provides non-transitory computer-readable medium computer readable carrier storing computer program code for individually (typically selectively) adjusting (typically alternating current (AC), typically current and/or voltage) electrical signals across or between each of the said electrodes of the electrode apparatus of the first aspect of the invention and each of one or more pairing electrodes.

A fifth aspect of the invention provides electrode apparatus for non-invasively applying (or configured to non-invasively apply) electrical stimulation to or detecting electrical signals from a body portion (typically to or from a target treatment region of a body portion internal to the body portion, such as a brain or a portion of the brain) of a human subject by way of a skin interface (e.g. a skin interface of the subject's scalp), the electrode apparatus comprising: an electrode module having: an (first) end for defining an electrolyte application region (typically comprising electrolyte in use) between the electrode module and the skin interface; and one or more electrodes which are electrically couplable or electrically coupled to the skin interface by way of an electrolyte in the said electrolyte application region; one or more electrolyte reservoirs containing electrolyte for electrically coupling the electrode(s) to the skin interface; and a controller configured to selectively dispense electrolyte from the electrolyte reservoir(s) to the electrolyte application region and/or to selectively remove electrolyte from the electrolyte application region.

It will be understood that typically, in use, electrolyte is provided in the electrolyte application region.

Typically the one or more electrolyte reservoirs are provided in or on the electrode module. By providing the reservoir(s) in or on the electrode module, a more compact and user friendly arrangement can be provided which makes the electrode apparatus more suitable for use outside of a laboratory or hospital environment (e.g. at the home of the human subject). By selectively dispensing electrolyte from the electrolyte reservoir(s) to and/or selectively removing electrolyte from the electrolyte application region, the correct quantity of electrolyte can be provided to the electrolyte application region, thereby reducing or preventing dry-spots from occurring within the electrolyte application region and preventing leakage of excess electrolyte from the electrolyte application region. This provides a more convenient apparatus for and reduces mess during the application of electrical stimulation to the human subject.

It may be that the controller is configured to employ a closed-loop control system to control the selective dispensation of electrolyte from the electrolyte reservoir(s) to, and/or the selective removal of electrolyte from, the electrolyte application region.

It may be that the controller (typically a or the closed loop control system) is provided with feedback, the controller being configured to selectively dispense electrolyte to, and/or selectively remove electrolyte from, the electrolyte application region responsive to the said feedback.

It may be that the feedback is indicative of a distribution of electrolyte in the electrolyte application region.

It may be that the feedback is indicative of a degree of contact between the electrolyte and one or more of the electrode(s) of the electrode module.

It may be that the feedback is indicative of a quantity of electrolyte in the electrolyte application region or at one or more (typically two or more) localised sub-regions of the electrolyte application region.

It may be that the feedback is indicative of (e.g. a magnitude of) a current flow through (e.g. current density in) each of one or more (typically each of two or more) localised sub-regions of the electrolyte application region. It may be that the feedback is indicative of a spatial current distribution within the electrolyte application region.

It may be that the feedback is indicative of one or more impedances or resistances of the electrolyte application region.

It may be that the feedback is indicative of impedances or resistances of one or more (typically two or more) localised sub-regions of the electrolyte application region. The feedback may comprise an estimated quantity of electrolyte to be added to and/or removed from the electrolyte application region, or to or from each of a plurality of localised sub-regions of the electrolyte application region. The said estimate may be derived from impedances or resistances of one or more (typically two or more) localised sub-regions of the electrolyte application region. The said estimate(s) may be derived from an (e.g. computer generated, typically mathematical, typically dynamically updated) impedance model of the impedance or resistance of each of the localised sub-regions (e.g. as a function of position).

It may be that the controller is configured to selectively dispense electrolyte from the electrolyte reservoir(s) to the electrolyte application region responsive to a determination from the said feedback that an impedance or resistance of the electrolyte application region is outside of an acceptable range.

Typically the controller is configured to selectively dispense electrolyte from the electrolyte reservoir(s) to, and/or selectively remove electrolyte from, the electrolyte application region, or to the said localised sub-region of the electrolyte application region, by way of one or more electrolyte delivery lines.

It may be that the controller is configured to dispense electrolyte from the electrolyte reservoir(s) to, and/or remove electrolyte from, the electrolyte application region by way of one or more (typically two or more) electrolyte ducts extending to or through the said end of the electrode module.

Typically the electrolyte ducts are configured to be in fluid communication with the electrolyte application region in use.

Typically a plurality of electrolyte ducts is provided. Typically, the electrolyte ducts are spaced from each other across the said end of the electrode module (typically in a direction having a component perpendicular to the line of shortest distance between the said end of the electrode module and a second end opposite the said end). It may be that the ducts are arranged in a concentric arrangement or in a spiral shape on the said end of the electrode module.

It may be that the controller is configured to selectively dispense electrolyte from the electrolyte reservoir(s) to, and/or to selectively remove electrolyte from, the electrolyte application region through each of the said electrolyte ducts individually.

Typically the controller is configured to selectively dispense electrolyte from the electrolyte reservoir(s) to, and/or to selectively remove electrolyte from, the electrolyte application region through each of a plurality of electrolyte ducts individually by opening an electronically controllable valve associated with that duct. It may be that the controller is configured to selectively restrict electrolyte from flowing from the electrolyte reservoir(s) to the electrolyte application region through each of the said plurality of electrolyte ducts individually by closing a or the said electronically controllable valve associated with that duct. Typically the said electronically controllable valves are operable to allow electrolyte to flow from the reservoir(s) to the electrolyte application region (or vice versa) through the ducts with which they are associated when they are in the open position, and to restrict electrolyte from flowing from the reservoir(s) to the electrolyte application region through the ducts with which they are associated when they are in the closed position.

It may be that the controller is configured to selectively dispense electrolyte from the electrolyte reservoir(s) to, and or to selectively remove electrolyte from, each of a plurality of localised sub-regions within the electrolyte application region individually.

It may be that the electrode module comprises a plurality of electrodes spaced from each other across the said end of the electrode module (typically in a direction perpendicular to the line of shortest distance between the said end of the electrode module and a second end of the electrode module opposite the first end or in a direction parallel to the line of shortest distance between the said end of the electrode module and a second end of the electrode module opposite the first end, or both).

It may be that the said plurality of electrodes comprises a two dimensional array of electrodes. It may be that the said plurality of electrodes comprises a three dimensional array of electrodes.

It may be that each of a plurality of the electrodes of the electrode module are provided adjacent to a different electrolyte duct.

It may be that the controller is configured to selectively dispense electrolyte from the electrolyte reservoir(s) to, and/or to selectively remove electrolyte from, a selected localised sub-region of the electrolyte application region through one or more of the said electrolyte ducts (e.g. by opening electronically controllable valves associated with the said one or more ducts), responsive to feedback specific to that localised sub-region (e.g. indicative of an impedance or resistance of that sub-region being outside an acceptable range).

It may be that the controller is configured to selectively dispense electrolyte from the reservoir(s) to, and/or to selectively remove electrolyte from, one or more selected localised sub-regions of the electrolyte application region (typically through one or more electrolyte ducts) responsive to feedback specific to those sub-regions (typically whilst not dispensing electrolyte into one or more other localised sub-regions of the electrolyte application region).

For example, it may be that the said feedback specific to a said sub-region provides an indication that there is insufficient, or too much, electrolyte in that sub-region respectively.

For example, the said feedback specific to the said sub-region may be indicative of an impedance or resistance between the skin interface and an electrode (or between two of the said electrodes) provided in the said localised sub-region.

It may be that the said feedback specific to a said sub-region is indicative of a current flow through (e.g. current density in) the said sub-region.

It will be understood that whether there is sufficient electrolyte in the electrolyte application region (or in one or more localised sub-regions of the electrolyte application region) may be determined by determining a difference between the measured impedances (e.g. of the localised sub-regions) of the electrolyte application region and a target impedance profile (e.g. a target impedance mathematical surface or volume) for the electrolyte application region.

It may be that the controller is configured to selectively dispense electrolyte into and/or selectively remove electrolyte from, the electrolyte application region by way of one or more electrolyte ducts, each of the electrolyte ducts being provided by a respective axial member extending to or through the said end of the electrode module.

Typically a plurality of electrolyte ducts are provided, each being provided by a respective axial member of a plurality of axial members extending to or through the said end of the electrode module.

Typically the axial members have longitudinal axes parallel to the line of shortest distance between the said end of the electrode module and a second end of the electrode module opposite the first end.

Typically the axial members are conical or frusto-conical in shape. Typically the narrower ends of the conical or frusto-conical axial members comprise the electrolyte ducts.

Typically the controller is configured to selectively dispense electrolyte into, and/or to selectively remove electrolyte from, each of a plurality of localised sub-regions of the electrolyte application region individually by way of an electrolyte duct of a said axial member provided in or adjacent to the said localised sub-region.

It may be that at least one of the said one or more electrodes is mounted to a said axial member.

It may be that the said one or more electrodes comprises a plurality of electrodes, each of which is mounted to a said different one of the said axial members.

It may be that one or more or each of the electrodes are annular. It may be that each of a plurality of (or each of) the annular electrodes is mounted to one of the said axial members by way of an annulus of the annular electrode (e.g. the annulus may receive the axial member).

Where the axial members are conical or frusto-conical, the annular electrodes are typically offset back from the electrolyte ducts. Typically the annular electrodes are mounted to sections of the conical or frusto-conical axial members having greater perimeters than the electrolyte ducts.

It may be that two or more (e.g. annular) electrodes are mounted to one axial member, the said two or more electrodes being offset from each other along an (e.g. longitudinal) axis of the axial member. It may be that two or more (e.g. annular) electrodes are mounted to each of a plurality of axial members, the said two or more electrodes being offset from each other along the axis of the axial member in each case.

Typically the electrodes comprise electrical conductors. It may be that the electrodes comprise metal. It may be that the electrodes comprise a conductive elastomer (e.g. elastomer comprising conductive material).

Typically the electrode apparatus comprises one or more electrolyte flow directors in (typically electrical) communication with the controller, the controller being configured to selectively dispense electrolyte from the electrolyte reservoir(s) to, and/or selectively remove electrolyte from, the electrolyte application region by activating one or more of the electrolyte flow directors or a respective one of the electrolyte flow directors.

It may be that the apparatus further comprises one or more electrolyte flow directors in (typically electrical) communication with the controller, the controller being configured to selectively dispense electrolyte from the electrolyte reservoir(s) to each of the electrolyte ducts individually by activating one or more of the electrolyte flow directors or a respective one of the electrolyte flow directors.

Typically the controller is configured to selectively and individually dispense electrolyte from each of the plurality of electrolyte application ducts by activating one or more of the electrolyte flow directors or a respective electrolyte flow director.

Each of the electrolyte flow directors may comprise a (e.g. air) pressure gradient generator configurable to selectively provide a (positive (to dispense electrolyte) or negative (to remove electrolyte)) pressure gradient between the reservoir and one or more of the said electrolyte application ducts. Typically the pressure gradient generator comprises a pump. However, it will be understood that any suitable alternative pressure gradient generator could be employed. For example the pressure gradient generator may comprise any one or more of: a piezo-electric pump; a pressurised gas reservoir configurable to selectively apply to the required pressure gradient; off-gassing from a chemical reaction; or gaseous expansion caused by heating. Alternatively, one or more of the electrolyte flow directors may comprise a selective gravity feed, which can be activated by way of one or more mechanical switches or one or more of the said electronically controlled valves.

It may be that the pressure gradient generator is operable to provide positive and negative pressure gradients in different operating modes. It may be that the controller is configured to control whether the pressure gradient generator provides a positive or negative pressure gradient (e.g. by controlling a mode of operation of the pressure gradient generator). It may be that the controller is configured to remove electrolyte from the electrolyte application region by way of one or more electrolyte ducts in communication with the electrolyte application region by applying a negative pressure gradient to the duct by way of the pressure gradient generator. It may be that the controller is configured to remove electrolyte from the electrolyte application region by way of each of a plurality of electrolyte ducts individually by applying a negative pressure gradient to the duct by way of the pressure gradient generator and by opening electronically controlled valves associated with those ducts. It may be that the pressure gradient generator is configured to direct the electrolyte removed from the electrolyte application region to one or more (e.g. one or more of the said) electrolyte reservoirs.

It may be that the controller is configured to dispense electrolyte to the electrolyte application region by way of one or more electrolyte ducts in communication with the electrolyte application region by applying a positive pressure gradient to the ducts by way of the pressure gradient generator. It may be that the controller is configured to dispense electrolyte from the electrolyte application region by way of each of a plurality of electrolyte ducts individually by applying a positive pressure gradient to the ducts by way of the pressure gradient generator and by opening electronically controlled valves associated with those ducts.

Typically the electrode module comprises the electrolyte flow directors.

It may be that the one or more electrolyte reservoirs are re-fillable.

Additionally or alternatively the one or more electrolyte reservoirs may be replaceable.

It may be that the one or more electrolyte reservoirs are disposable.

It may be that the controller is provided in the electrode module. More typically the controller is distributed between a plurality of locations. It may be that at least part of the controller is provided in the electrode module. It may be that part of the controller is provided outside of the electrode module. It may be that the controller is implemented in hardware or in software, but more typically the controller is implemented in a combination of hardware and software.

A sixth aspect of the invention provides a method of non-invasively applying electrical stimulation to or detecting electrical signals from a body portion (typically to or from a target treatment region of a body portion internal to the body portion, such as a brain or a portion of the brain) of a human subject by way of a skin interface (e.g. a skin interface of the subject's scalp), the method comprising: defining an electrolyte application region (typically comprising electrolyte in use) between an end of an electrode module and the skin interface, the said electrode module comprising one or more electrodes; providing one or more electrolyte reservoirs containing electrolyte for electrically coupling the electrode(s) to the skin interface; and electrically coupling the said one or more electrodes to the skin interface by selectively dispensing electrolyte from the electrolyte reservoir(s) to the electrolyte application region.

A seventh aspect of the invention provides electrode apparatus for non-invasively applying (or configured to non-invasively apply) electrical stimulation to or detecting electrical signals from a body portion (typically to or from a target treatment region of a body portion internal to the body portion, such as a brain or a portion of the brain) of a human subject by way of a skin interface (e.g. a skin interface of the subject's scalp), the electrode apparatus comprising: an electrode module having: an (first) end for defining (or which defines) an electrolyte application region (typically comprising electrolyte in use) between the electrode module and the skin interface; one or more electrodes which are electrically couplable or electrically coupled to the skin interface by way of an electrolyte in the said electrolyte application region; and electrolyte containment apparatus configured to restrict leakage of electrolyte from the electrolyte application region.

It will be understood that typically, in use, electrolyte is provided in the electrolyte application region.

By providing electrolyte containment apparatus in the electrode module, electrolyte leakage is reduced (or even eliminated) which makes the electrode apparatus more suitable (and convenient) for use outside of a controlled laboratory or hospital environment (e.g. at a home of the human subject).

It may be that the electrolyte containment apparatus comprises an electrolyte absorber provided on the said (first) end of the electrode module.

It may be that the electrode module comprises a plurality of electrodes electrically couplable or electrically coupled to the skin interface by way of an electrolyte in the said electrolyte application region.

It may be that the electrolyte absorber at least partially (preferably fully) surrounds at least some of (preferably all of) the electrodes of the electrode module.

Typically the electrolyte absorber is provided around at least part of the (preferably the entire) perimeter of the said (first) end of the electrode module (e.g. at an edge of the said end of the electrode module).

It may be that the electrolyte containment apparatus comprises a seal provided on the said (first) end of the electrode module for restricting leakage of electrolyte from the electrolyte application region.

Typically the seal extends around at least part of (preferably the entire) perimeter of the said (first) end of the electrode module. Typically the seal is configured to form a seal between the said end of the electrode module and the skin interface to restrict leakage of electrolyte from the electrolyte application region.

It may be that the electrolyte containment apparatus comprises a pressure gradient generator in fluid communication with the said (first) end of the electrode module (e.g. by way of one or more holes in the said (first) end of the electrode module) for restricting leakage of electrolyte from the electrolyte application region.

It may be that the pressure gradient generator is configured or configurable to apply a negative pressure gradient between the electrode module (e.g. including one or more electrolyte reservoirs provided in the electrode module) and the said (first) end of the electrode module so as to restrict leakage of electrolyte from the electrolyte application region.

It may be that the pressure gradient generator comprises a (e.g. mechanical) pump. It may be that the pressure gradient generator comprises a vacuum pump. It may be that the pressure gradient generator employs any one of: a static pressure reservoir; gas absorption from a chemical reaction; gaseous contraction caused by cooling; or a capillary feed.

Preferably the pressure gradient generator is configured to direct electrolyte from the electrolyte application region to one or more electrolyte reservoirs (typically provided in the electrode module) for later re-use.

It may be that the electrode apparatus comprises a controller in communication with the pressure gradient generator for selectively applying a (e.g. negative) pressure gradient to restrict leakage of electrolyte from the electrolyte application region. It may be that the electrode apparatus comprises a user control for selectively applying a (e.g. negative) pressure gradient to restrict leakage of electrolyte from the electrolyte application region.

It may be that the electrolyte containment apparatus comprises a porous seal provided on the said (first) end of the electrode module for restricting leakage of electrolyte from the electrolyte application region and a pressure gradient generator in communication with the said seal, the pressure gradient generator configured or configurable to apply a (typically negative) pressure gradient between one or more holes in the porous seal and the electrode module (typically including an electrolyte reservoir of the electrode module) to thereby restrict leakage of electrolyte from the electrolyte application region.

It may be that the electrolyte containment apparatus comprises a plurality of walls provided at (typically extending from a surface of) the (first) end of the electrode module, the said walls defining localised sub-regions within the electrolyte application region and being configured to restrict electrolyte (and typically current) leakage from the said localised sub-regions when the said (first) end of the electrode module is installed on the skin interface.

It may be that each of the localised sub-regions comprises one or more electrodes of the electrode module.

It may be that each of the localised sub-regions comprises one or more (e.g. a single) axial member on which one or more electrodes of the electrode module are mounted.

It may be that each of the localised sub-regions comprises one or more (e.g. a single) electrolyte duct through which electrolyte can be dispensed into the localised sub-region.

By restricting electrolyte and current leakage from localised sub-regions within the electrolyte application region, the current density in each of the localised sub-regions can be more easily controlled.

An eighth aspect of the invention provides a method of non-invasively applying (or configured to non-invasively apply) electrical stimulation to or detecting electrical signals from a body portion (typically to or from a target treatment region of a body portion internal to the body portion, such as a brain or a portion of the brain) of a human subject by way of a skin interface (e.g. a skin interface of the subject's scalp), the method comprising: defining an electrolyte application region (typically comprising electrolyte in use) between an end of an electrode module and the skin interface, the said electrode module comprising one or more electrodes; electrically coupling the said electrode(s) to the skin interface by way of an electrolyte provided in the said electrolyte application region; and restricting leakage of electrolyte from the electrolyte application region.

A ninth aspect of the invention provides electrode apparatus for non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the electrode apparatus comprising: an electrode module having: an (first) end for defining an electrolyte application region (typically comprising electrolyte in use) between the electrode module and the skin interface; and one or more electrodes which are electrically couplable or electrically coupled to the skin interface by way of an electrolyte in the said electrolyte application region; a controller configured to apply electrical stimulation to the body portion by way of the one or more electrodes; and one or more sensors configured to measure one or more physiological stress indicators indicative of a physiological stress of the human subject (typically the said physiological indicators being responsive to the electrical stimulation applied to the subject by way of the electrodes), wherein the controller is further configured to: receive the said one or more measured stress indicators from the said sensors; determine whether one or more physiological stress criteria are met taking into account the measured physiological stress indicators; and provide an output responsive to a determination that the said physiological stress criteria are met.

It will be understood that typically, in use, electrolyte is provided in the electrolyte application region.

By (typically automatically) detecting one or more physiological stress indicators indicative of a physiological stress of the human subject, action can be taken to reduce or prevent any discomfort experienced by the human subject during an application of electrical stimulation. While this is suitable for a controlled application environment such as a laboratory or hospital, it is particularly suitable for use of the electrode apparatus away from medical supervision, such as in the home of the subject.

It may be that one or more or each of the sensors are provided in or on (e.g. the said end of) the electrode module.

It may be that one or more or each of the sensors are couplable or coupled to the body portion.

It may be that one or more or each of the sensors are couplable or coupled to one or more second body portions of the human subject different from the said body portion.

It may be that one or more or each of the sensors are comprised in a hand-held or wearable electronic device of the human subject (e.g. a personal, typically portable, electronic communications device of the human subject).

It may be that the one or more sensors comprise one or more or each of the electrodes of the electrode module. For example, the controller may be configured to use one or more electrodes of the electrode module in an electroencephalography (EEG) mode in order to measure one or more physiological stress indicators indicative of a physiological stress of the human subject. EEG can be used, for example, to detect the onset of a migraine in the human subject (e.g. by detecting an aura).

It may be that the electrode apparatus further comprises an input device (e.g. a personal, typically portable, electronic communications device of the human subject) by which the human subject can manually enter one or more physiological stress indicators (which are typically taken into account by the controller when determining whether the said physiological stress criteria are met).

It may be that the electrode apparatus comprises a plurality of sensors spaced from each other at the said end of the electrode module (typically in a direction having a component perpendicular to a line of shortest distance between the said end of the electrode module and a second end of the electrode module opposite the said (first) end), each of the sensors being configured to measure (the same or different) respective physiological stress indicators of the subject.

It may be that the electrode apparatus comprises a plurality of sensors, each of which is configured to measure a physiological stress indicator at (e.g. specific to) a different localised sub-region of the electrolyte application region.

It may be that the controller is configured to determine a value of a function taking into account the measured physiological stress indicators. It may be that the controller is configured to determine that the physiological stress criteria are met if the determined value of the function is outside of an acceptable range (e.g. beyond a limit).

It may be that the controller is configured to determine whether each of the measured physiological stress indicators meets one or more respective physiological stress criteria and to determine that the physiological stress criteria are met responsive to a determination that one or more (or two or more or each) of the measured physiological stress indicators meet the said respective physiological stress criteria.

It may be that the electrode apparatus comprises first and second sensors, the first sensor being configured to measure a first said physiological stress indicator of the human subject and the second sensor being configured to measure a second said physiological stress indicator of the human subject different from the first physiological stress indicator.

It may be that the first said physiological stress indicator is an indicator of a first physiological stress of the subject and the second said physiological stress indicator is an indicator of a second physiological stress of the subject different from the first physiological stress.

It may be that the output provided responsive to the determination that the said physiological stress criteria are met comprises one or more signals which cause a visual, audible and/or tactile notification (e.g. a notification that the said physiological stress criteria are met), such as a warning or an alarm.

It may be that the output provided responsive to the determination that the said physiological stress criteria are met comprises one or more signals for reducing the physiological stress of the human subject.

It may be that the output provided responsive to the determination that the said physiological stress criteria are met comprises one or more signals which adjust the electrical stimulation applied to the body portion by way of the one or more electrodes.

Typically the said electrical stimulation applied to the body portion by way of the one or more electrodes is adjusted to thereby reduce the said physiological stress of the human subject.

It may be that the electrical stimulation applied to the body portion is adjusted by reducing the amplitude of the electrical signals applied to one or more of the electrodes.

It may be that the controller is configured to adjust the electrical stimulation applied to the body portion by adjusting electrical signals applied to each of two or more electrodes.

It may be that the electrode apparatus comprises a plurality of electrodes spaced apart from each other (typically across the electrolyte application region in use) and wherein the controller is configured to adjust the electrical stimulation applied to the body portion by adjusting electrical signals applied to each of two or more of the electrodes.

For example, it may be that the controller is configured to increase a current carried by a first electrode of the electrode module and to decrease a current carried by a second electrode of the electrode module or vice versa.

In another example the electrode apparatus comprises a second electrode module having: an (first) end for defining a second electrolyte application region (typically comprising electrolyte in use) between the electrode module and the skin interface; one or more electrodes which are electrically couplable or electrically coupled to the skin interface by way of an electrolyte in the said second electrolyte application region, the electrodes being in communication with the controller. In this case, it may be that the controller is configured to increase a current carried by the electrodes of the (first) electrode module (as a whole) and to decrease a current carried by the electrodes of the second electrode module (as a whole) or vice versa. It will be understood that typically, in use, electrolyte is provided in the second electrolyte application region.

It may be that the controller is configured to reduce the physiological stress of the subject by adjusting a current distribution between electrodes of the electrode module, or by adjusting a current distribution between the electrode module and the second electrode module, responsive to the determination that the one or more physiological stress criteria are met (e.g. a function of the measured physiological stress indicators has a value which is outside of an acceptable range).

It may be that the controller is configured to adjust the electrical stimulation applied to the body portion by adjusting any one or more of the following aspects of the electrical signals applied to one or more of the electrodes: the waveform; frequency content; and polarisation (e.g. by applying a DC offset).

It may be that the output provided responsive to the determination that the said physiological stress criteria are met comprises a signal which causes electrical stimulation being applied to the body portion to be aborted.

It may be that the output provided responsive to the determination that the said physiological stress criteria are met comprises a signal which causes electrolyte to be selectively dispensed to the electrolyte application region.

It may be that the output provided responsive to a determination that first physiological stress criteria are met comprises a signal which causes electrolyte to be selectively dispensed to the electrolyte application region or a notification to be provided and the output provided responsive to a determination that second physiological stress criteria different from the first physiological stress criteria are met comprises a signal which causes the electrical stimulation applied to the body portion by the electrodes to be adjusted (e.g. reduced).

It may be that each of one or more (typically each of two or more) of the said sensors are configured to measure a physiological stress indicator specific to a respective localised sub-region of the electrolyte application region, wherein the controller is configured to determine whether one or more localised physiological stress criteria are met taking into account the measured physiological stress indicator and to provide an output specific to the said localised sub-region responsive to a determination that said one or more localised physiological stress criteria specific to that sub-region are met.

It may be that the said one or more sensors comprise one or more sensors configured to measure a physiological stress indicator which comprises a physiological parameter of the body portion (e.g. on the skin interface).

It may be that the one or more sensors comprise one or more sensors configured to measure one or more physiological stress indicators indicative of a skin sensitivity of the human subject.

It may be that the said one or more sensors comprise one or more colourimeters configured to measure a parameter indicative of a colour of the body portion (e.g. of the skin interface).

For example the said one or more sensors may comprise one or more light sources (e.g. laser or LED) and one or more light detectors (e.g. photodiode, phototransistor, image detector such as a camera or infrared camera) configured to detect light of a wavelength emitted by the light source. Typically the light source is configured to emit light towards the skin interface. Typically the light detector is configured to detect light emitted by the light source which has been reflected from the skin interface.

It may be that the said one or more colourimeters are configured to measure a parameter indicative of a red or infrared colour of the body portion (e.g. of the skin interface).

The said light source(s) may comprise a light source configured to emit light having a wavelength in the region 620 nm to 750 nm (red light), or in the infrared region. This allows the colourimeter to measure a parameter indicative of the redness of the skin interface, which is a useful (and typically reliable) indicator of the physiological stress of the subject.

It may be that skin redness is a pre-cursor to skin lesions forming. Accordingly, it may be that the colourimter is a sensor configured to determine a physiological stress indicator (e.g. redness of the skin) indicative of a pre-ictal state of the human subject (e.g. redness of the skin may be a pre-cursor to skin lesions forming).

It may be that the said one or more sensors comprise a pH sensor configured to measure a pH of the skin interface.

It may be that the said one or more sensors comprise a temperature sensor configured to measure a temperature of the skin interface.

It may be that the one or more sensors comprise one or more sensors configured to measure one or more physiological stress indicators indicative of a pre-ictal state of the subject.

It may be that the one or more sensors comprise one or more sensors configured to measure one or more physiological stress indicators indicative of a precursor to fit, migraine or skin lesion.

For example, the one or more sensors may comprise one or more blood pressure sensors configured to determine the blood pressure of the human subject.

Additionally or alternatively, the one or more sensors may comprise one or more heart rate monitors configured to determine the heart rate (or changes in the heart rate) of the human subject.

Additionally or alternatively, the one or more sensors may comprise one or more movement sensors (e.g. accelerometer, gyroscope) configured to detect movements indicative of a pre-ictal state of the human subject (e.g. pre-epileptic fit).

Additionally or alternatively, the one or more sensors may comprise one or more sensors of blood oxygen saturation (such as a pulse oximeter). It may be that the blood oxygen saturation sensor is configured to determine changes in blood oxygen saturation levels indicative of a pre-ictal state of the subject (e.g. changes in blood oxygen saturation levels may be a pre-cursor to a fit).

By detecting one or more physiological stress indicators indicative of a pre-ictal state of the human subject, corrective action can be taken before the human subject experiences discomfort.

It may be that the one or more sensors comprise one or more movement sensors.

For example, it may be that the said one or more movement sensors comprise any one or more of the following: accelerometer; gyroscope; magnetometer. By detecting movements which are indicative of a physiological stress (e.g. slumping, shaking, seizure, having a fit) of the subject, it can be determined whether the subject is experiencing a said physiological stress.

The one or more movement sensors may comprise one or more sensors for indirectly detecting movements which are indicative of a physiological stress (e.g. slumping, shaking, seizure, having a fit) of the subject. For example, the movement sensors may comprise any one or more of: heart rate monitor; heart rate variability oximeter; blood pressure detector; temperature sensor; and an electroencephalogram (EEG).

It may be that the one or more sensors comprise one or more or each of the electrodes of the electrode module configured to operate in an electroencephalography (EEG) mode.

For example, the controller may be configured to use one or more electrodes of the electrode module in an electroencephalography (EEG) mode in order to measure one or more physiological stress indicators indicative of the onset of a migraine in the human subject (e.g. by detecting an aura).

Detection of a migraine aura from the electrodes in EEG mode (see above) may also be considered to be detection of a pre-ictal state of the subject (i.e. pre-cursor to migraine).

It may be that the said one or more sensors are in (e.g. wired or more preferably wireless) data communication with the controller.

It may be that the controller is provided in the electrode module. More typically the controller is distributed between a plurality of locations. It may be that at least part of the controller is provided in the electrode module. It may be that part of the controller is provided outside of the electrode module. It may be that the controller is implemented in hardware or in software, but more typically the controller is implemented in a combination of hardware and software.

A tenth aspect of the invention provides a method of non-invasively applying (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the method comprising: defining an electrolyte application region (typically comprising electrolyte in use) between an end of an electrode module and the skin interface, the electrode module comprising one or more electrodes; electrically coupling the one or more electrodes to the skin interface by way of an electrolyte provided in the said electrolyte application region; applying electrical stimulation to the body portion by way of the electrode(s); measuring one or more physiological stress indicators indicative of a physiological stress of the human subject (typically the said physiological stress being responsive to, and/or caused by, electrical stimulation applied to the subject by way of the electrodes); determining whether one or more physiological stress criteria are met taking into account the measured physiological stress indicators; and providing an output responsive to a determination that the said physiological stress criteria are met.

An eleventh aspect of the invention provides electrode apparatus for non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the electrode apparatus comprising: a first electrode module having: an (first) end for defining a first electrolyte application region between the first electrode module and the skin interface, the first electrode module comprising one or more electrodes which are electrically couplable or electrically coupled to the skin interface by way of an electrolyte in the said first electrolyte application region; a second electrode module having: an (first) end for defining a second electrolyte application region between the second electrode module and the skin interface, the second electrode module comprising one or more electrodes which are electrically couplable or electrically coupled to the skin interface by way of an electrolyte in the said second electrolyte application region; one or more shunt measurement conductors; and a controller configured to: determine (e.g. measure) one or more electrical parameters (e.g. voltage and/or current and/or impedance or resistance) between one or more electrodes of the first electrode module and one or more of the shunt measurement conductors; and determine a current shunted across the skin interface between the first and second electrode modules taking into account the said one or more determined electrical parameters.

It will be understood that typically, in use, electrolyte is provided in the first and second electrolyte application regions.

Typically the controller is configured to (typically selectively, typically individually) adjust electrical signals across or between one or more of the electrodes of the first electrode module and one or more electrodes of the second electrode module.

Typically the controller is configured to determine the current shunted across the skin interface between the first and second electrode modules in response to electrical signals applied between the electrode(s) of the first and second electrode modules taking into account the said one or more determined (e.g. measured) electrical parameters.

Typically the first electrode module comprises one or more or each of the shunt measurement conductors.

It may be that one or more or each of the shunt measurement conductors are provided on or adjacent to the said (first) end of the first electrode module.

Typically one or more or each of the said one or more shunt measurement conductors are configured to be provided in the first electrolyte application region.

It may be that one or more or each of the shunt measurement conductors are provided between the electrode(s) of the first electrode module and an edge of the said (first) end of the first electrode module.

Typically the edge of the said (first) end of the first electrode module is provided at or adjacent to the perimeter of the said (first) end of the first electrode module.

Typically the one or more shunt measurement conductors are provided closer to the edge (e.g. the perimeter) of the said (first) end of the first electrode module than the said electrode(s) of the first electrode module are to the said edge.

It may be that one or more or each of the shunt measurement conductors are provided around the one or more electrodes of the first electrode module (or at least projected positions of the electrode(s) onto a plane comprising the said shunt measurement conductors) in a curved, arced, semi-circular or circular arrangement.

It may be that one or more or each of the said shunt measurement conductors substantially surround the electrode(s) of the first electrode module (or at least projected positions of the electrode(s) of the first electrode module onto a plane comprising the said shunt measurement conductors) in two dimensions.

It may be that the controller is configured to: apply one or more electrical (typically AC) test signals (typically an electrical current) between one or more electrodes of the first electrode module and one or more of the shunt measurement conductors; determine (e.g. measure) one or more electrical parameters across or between (typically a voltage across) the said electrodes of the first electrode module and the said shunt measurement conductors responsive to the said test signal; and determine the said current shunted across the skin interface between the first and second electrode modules taking into account the said one or more determined (e.g. measured) electrical parameters.

Typically the controller is configured to determine an impedance of an electrical path between the said electrodes of the first electrode module and the said shunt measurement conductors from the said determined (e.g. measured) electrical parameters, and to determine the said current shunted across the skin interface between the first and second electrode modules taking into account the said determined impedance.

It may be that the test signals are superimposed on electrical stimulation signals applied between the electrodes of the first and second electrode modules. The test signals may be applied by, for example, increasing or decreasing (e.g. amplitudes of) electrical stimulation signals applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed.

Alternatively, the test signals may be applied in the absence of electrical stimulation signals between the electrodes of the first and second electrode modules.

It may be that the controller is configured to: apply (second) electrical (typically AC, typically current) test signals between the said electrodes of the first electrode module and the said electrodes of the second electrode module; determine (e.g. measure) one or more electrical parameters (e.g. voltage and/or current) between the said one or more electrodes of the first electrode module and the one or more electrodes of the second electrode module; and determine the said current shunted across the skin interface between the first and second electrode modules further taking into account the said one or more electrical parameters (e.g. voltage and/or current and/or impedance or resistance) determined (e.g. measured) across or between the said one or more electrodes of the first electrode module and the one or more electrodes of the second electrode module.

It may be that the (second) test signals are superimposed on electrical stimulation signals applied between the electrodes of the first and second electrode modules. The second test signals may be applied by, for example, increasing or decreasing (e.g. amplitudes of) electrical stimulation signals applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed.

Alternatively, the second test signals may be applied in the absence of electrical stimulation signals between the electrodes of the first and second electrode modules.

It may be that the (first) test signals are applied between the said electrodes of the first electrode module and the said shunt measurement conductors prior to or after the (second) test signals applied across or between one or more of the electrodes of the first electrode module and one or more electrodes of the second electrode module.

It may be that the one or more shunt measurement conductors comprises a plurality of shunt measurement conductors spaced apart from each other (typically such that one or more electrical current paths are provided across the skin interface between the said one or more electrodes and the said one or more pairing electrodes which does not pass through any of the one or more shunt measurement conductors).

By spacing the shunt measurement conductors apart from each other, the shunt measurement conductors can be made smaller in size (while still spreading out over a given surface area) to thereby reduce the effect of the shunt measurement conductors on the current shunted along the skin interface from the electrodes can be reduced.

Typically the plurality of shunt measurement conductors comprises a plurality of shunt measurement conductors spaced apart from each other adjacent to the said edge of the said (first) end of the first electrode module (e.g. a plurality of shunt measurement conductors spaced apart from each other adjacent to the said edge of the said (first) end of the first electrode module).

It may be that each of a plurality of the shunt measurement conductors are spaced equally from the said one or more electrodes of the first electrode module.

It may be that the one or more shunt measurement conductors comprises one or more first shunt measurement conductors and one or more second shunt measurement conductors, the first shunt measurement conductors being positioned closer to the electrodes of the first electrode module than the second shunt measurement conductors are to the electrodes of the first electrode module.

Typically the controller is configured to measure one or more electrical parameters at (one or more or each or all of) the first shunt measurement conductors distinctly from (one or more or each or all of) the second shunt measurement conductors.

Typically the first and second shunt measurement conductors are arranged such that one or more first shunt measurement conductors and one or more second shunt measurement conductors can both detect a current shunted along the skin interface in response to electrical signals applied between electrodes of the first and second electrode modules.

Typically the controller is configured to determine the direction of a current shunted across the skin interface by determining (e.g. measuring) one or more electrical parameters (e.g. current flowing between) between the first and second shunt measurement conductors.

It may be that the controller is configured to determine the said current shunted across the skin interface of the said body portion by measuring an electrical parameter (e.g. current flowing between or voltage across) between or across one or more of the first shunt measurement conductors and one or more of the second shunt measurement conductors.

Typically the one or more first shunt measurement conductors and the one or more second shunt measurement conductors are provided in curved, arced, semi-circular or circular arrangements (typically around the electrodes of the first electrode module).

Typically the one or more first shunt measurement conductors comprises a first plurality of shunt measurement conductors and the one or more second shunt measurement conductors comprises a second plurality of shunt measurement conductors.

Typically, within each of the first and second pluralities of shunt measurement conductors, the shunt measurement conductors are spaced apart from each other (typically such that one or more electrical current paths are provided across the skin interface between the first and second electrode modules which do not pass through any of the one or more shunt measurement conductors of the said first and second pluralities).

Typically one or more of the first shunt measurement conductors and one or more of the second shunt measurement conductors are provided on a straight line extending between one or more of the electrodes of the first electrode module and an edge of the said (first) end of the first electrode module (typically along the (first) end of the first electrode module).

It may be that the controller is configured to estimate a dosage of electrical stimulation impinging on a or the target treatment region of (typically internal to) the body portion in response to electrical signals applied between the electrode(s) of the first and second electrode modules taking into account the determined current shunted across the skin interface.

The current shunted across the skin interface can be used to more accurately determine a dosage of electrical stimulation impinging on a target treatment region of the body portion (e.g. internal to the body portion). This helps to improve safety, and to ensure that an accurate dosage is applied (e.g. in accordance with a dosage regime) to the target treatment region of the body portion.

It may be that the second electrode module comprises one or more of the said shunt measurement conductor(s).

Typically the one or more shunt measurement conductors of the selected electrode module are provided on or adjacent to the said (first) end of the second electrode module. Typically the one or more shunt measurement conductors of the second electrode module are provided between the electrode(s) of the second electrode module and an edge of the said (first) end of the second electrode module. Typically the edge of the said (first) end of the second electrode module is provided at or adjacent to the perimeter of the said (first) end of the second electrode module. The shunt measurement conductor(s) of the second electrode module may have any of the features of the shunt measurement conductor(s) of the first electrode module.

It may be that the controller is configured to determine (e.g. measure) one or more electrical parameters (e.g. voltage and/or current and/or impedance or resistance) between one or more electrodes of the second electrode module and the shunt measurement conductors of the second electrode module; and determine the said current shunted across the skin interface between the first and second electrode modules taking into account the said one or more determined (e.g. measured) electrical parameters between one or more electrodes of the second electrode module and the shunt measurement conductors of the second electrode module.

Typically the controller is configured to: apply one or more electrical (typically AC) (third) test signals (typically an electrical current) between one or more electrodes of the second electrode module and one or more of the shunt measurement conductors of the second electrode module; determine (e.g. measure) one or more electrical parameters across or between (typically a voltage across) the said electrodes of the second electrode module and the said shunt measurement conductors of the second electrode module responsive to the said (third) test signal; and determine the said current shunted across the skin interface between the first and second electrode modules taking into account the said one or more determined (e.g. measured) electrical parameters across or between the said electrodes of the second electrode module and the said shunt measurement conductors of the second electrode module.

It may be that the (third) test signals are applied across or between the said electrodes of the second electrode module and the said shunt measurement conductors of the second electrode module prior to or after the (first) test signals applied across or between the said electrodes of the first electrode module and the said shunt measurement conductors and prior to or after the (second) test signals applied across or between one or more of the electrodes of the first electrode module and one or more electrodes of the second electrode module.

Typically the controller is configured to determine an impedance of the electrical path between the said electrode(s) of the second electrode module and the said shunt measurement conductor(s) of the second electrode module from the said measured electrical parameter(s), and to determine the said current shunted across the skin interface between the first and second electrode modules taking into account the said determined impedance.

It may be that the (third) test signals are superimposed on electrical stimulation signals applied between the electrodes of the first and second electrode modules. The test signals may be applied by, for example, increasing or decreasing (e.g. amplitudes of) electrical stimulation signals applied between the electrodes of the first and second electrode modules. It may be that the electrical signals on which the test signals are superimposed comprise electrical signals providing a therapeutic dosage of electrical stimulation to the body portion. By superimposing test signals on electrical stimulation signals (e.g. already being) applied between the electrodes of the first and second electrode modules, the electrical stimulation treatment does not need to be stopped in order for the test signal measurements to be performed.

Alternatively, the (third) test signals may be applied in the absence of electrical stimulation signals between the electrodes of the first and second electrode modules.

It may be that the controller is configured to measure electrical signals (e.g. voltage, current) between one or more shunt measurement conductors of the first electrode module and one or more shunt measurement conductors of the second electrode module.

It may be that the first and second electrode modules each comprise one or more shunt measurement conductor(s), and wherein the controller is configured to determine the said current shunted across the skin interface of the said body portion taking into account one or more electrical parameters (e.g. voltage and/or current and/or impedance or resistance) determined (e.g. measured) across or between one or more shunt measurement conductors of the first electrode module and one or more shunt measurement conductors of the second electrode module.

It may be that the controller is configured to determine multiple values for the current shunted across the skin interface between the said one or more electrodes of the first and second electrode modules. It may be that the controller is configured to determine an average (e.g. mean) value from the said multiple values. It may be that the controller is configured to discard outlier values prior to any averaging (that is, it may be that the controller is configured to not include outlier values in the average value).

It may be that the controller is configured to adjust electrical signals applied to one or more electrodes of one or both of the first and second electrode modules (typically to thereby adjust the shape the electric field impinging on the target treatment region of the body portion by the electrodes, typically responsive to the said determined current shunted across the skin interface between the said one or more electrodes of the first and second electrode modules exceeding a threshold) to thereby reduce the current shunted across the skin interface between the first and second electrode modules.

This helps to provide a more targeted dosage of electrical stimulation to the target treatment region of the body portion, and helps to reduce irritation to the skin interface.

A twelfth aspect of the invention provides a method of non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the method comprising: defining a first electrolyte application region between an end of a first electrode module and the skin interface, the first electrode module comprising one or more electrodes; electrically coupling the said one or more electrodes of the first electrode module to the skin interface by providing an electrolyte in the said first electrolyte application region; defining a second electrolyte application region between an end of a second electrode module and the skin interface, the second electrode module comprising one or more electrodes; electrically coupling the said one or more electrodes of the second electrode module to the skin interface by providing an electrolyte in the said first electrolyte application region; providing one or more shunt measurement conductors; measuring one or more electrical parameters (e.g. current, potential) between one or more electrodes of the first electrode module and one or more of the shunt measurement conductors; and determining a current shunted across the skin interface between the first and second electrode modules taking into account the said one or more measured electrical parameters.

A thirteenth aspect of the invention provides data processing apparatus comprising a computer processor, the data processing apparatus being configured to: receive geometry data representing a geometry of a human body portion (e.g. a human head or a portion of a human head) comprising a target treatment region internal to the body portion (for example the target treatment region comprising a human brain or a portion of a human brain); receive impedance data indicative of one or more (typically electrical) impedances or resistances (typically data indicative of impedances or resistances of two or more different types of human tissue, such as skin, bone, brain, portions of the brain) of the said body portion; determine electric field data representing an (typically three dimensional) electrical field through the body portion, which is responsive to an electrical stimulation applied to the body portion by way of a skin interface of the body portion, taking into account the geometry data and the impedance data; and determine a dosage of electrical stimulation impinging on the target treatment region from the electric field data.

By taking into account the geometry data and the impedance data in the determination of the electric field applied through the body portion, the dosage of electrical stimulation impinging on the target treatment region (e.g. per unit stimulation applied to the skin interface of the body portion) can be determined more accurately. It can thus be better ensured that a safe dosage of electrical stimulation is impinging on the target treatment region at all times. It can also be determined whether the electrical stimulation impinging on the target treatment region is in accordance with an intended dosage regime.

It will be understood that the data processing apparatus is configured to apply said electrical stimulation to the said body portion (e.g. in parallel with the determination of the said electric field data representing the said electrical field through the body portion) by way of one or more electrodes in electrical communication with the skin interface (e.g. by way of an electrolyte), such that the said electric field data represents an estimate of the electrical field through the body portion responsive to the electrical stimulation applied to the body portion, and the determined dosage is an estimate of the dosage actually impinging on the target treatment region.

It may be that the geometry data comprises a mathematical model and/or image of the body portion.

It may be that the geometry data represents a three dimensional geometry of the human body portion.

For example it may be that the geometry data comprises one or more concentric spheres representing the human head. The geometry data may comprise two or more concentric spheres (e.g. three or four concentric spheres), each sphere representing a different portion of the human head (e.g. brain, skull, scalp).

Alternatively, it may be that the geometry data comprises an image of the body portion obtained by any one of magnetic resonance imaging, computed tomography, electrical impedance tomography, electrical impedance spectroscopy.

It may be that the geometry data is specific to a human subject comprising the said body portion. In other cases, it may be that the geometry data is not specific to a human subject.

It may be that the geometry data represents a geometry of both an external portion of the body portion and an internal portion of the body portion.

For example, the geometry data may represent a geometry of a scalp of a human head and a brain internal to the human head.

It may be that the impedance data comprises data indicative of (typically electrical) an impedance or resistance of a first type of human tissue external to the body portion and data indicative of an impedance or resistance of a second type of human tissue internal to the body portion.

Typically the impedance data comprises data indicative of impedances or resistances of different types of human tissue of along an electrical transmission path through the body portion (e.g. between two or more reference positions, the reference positions typically being on an external surface of the body portion).

It may be that the data processing apparatus is further configured to: determine electric field data representing an electrical field applied through the body portion responsive to an electrical stimulation applied to the body portion by way of a skin interface of the body portion taking into account the geometry data and the impedance data by: using the said geometry data and the impedance data to mathematically model (typically using Maxwell's equations) an electric field through the body portion responsive to the said electrical stimulation (e.g. as a function of position).

Typically the data processing apparatus is configured to mathematically model (e.g. using Maxwell's equations) the electric field applied through the body portion as a function of position responsive to the said electrical stimulation using two or more reference positions, each representing a position of an electrode module (or one or more electrodes of an electrode module) on the body portion to and from which the electrical stimulation is provided by way of the skin interface. Typically the data processing apparatus is configured to use the said reference positions in the mathematical modelling process.

Typically the data processing apparatus is configured to determine the said electric field data taking into account a geometry of the electrode module(s). For example, the data processing apparatus is configured to determine the said electric field data taking into account a surface area of the electrodes of the electrode modules in contact with the skin interface.

It may be that the data processing apparatus is configured to determine a (e.g. instantaneous) dosage of electrical stimulation impinging on the target treatment region responsive to the electrical stimulation by: determining an (typically mathematical, typically three dimensional) impedance model indicative of the impedance or resistance of the body portion as a function of position from the said geometry data and the said impedance data; and using the said impedance model to determine the dosage of electrical stimulation impinging on the target treatment region (e.g. by deriving the electric field data from the impedance model and determining the dosage of stimulation applied to the target treatment region from the electric field data).

It may be that the impedance model is not specific to the said human subject, but preferably the impedance model is specific to the human subject. It may be that the data processing apparatus is configured to receive said geometry data specific to the human subject and it may be that the data processing apparatus is configured to use the geometry data to determine the impedance model.

It may be that the data processing apparatus is further configured to determine a dosage of electrical stimulation impinging on the target treatment region from the determined electric field data using predetermined data indicative of the position of the target treatment region within the body portion.

For example, the target treatment region may comprise a portion of a human brain internal to a head portion of a human body. In this case, it may be that the predetermined data indicative of the position of the target treatment region within the body portion may comprise data (e.g. a mathematical model or image) indicative of the typical position of the said portion of the human brain within the human brain.

It may be that the data processing apparatus is further configured to: provide electrical signals between an electrode (e.g. of an electrode module) and a pairing electrode to thereby apply electrical stimulation to the body portion by way of the skin interface; determine electric field data representing the electrical field applied through the body portion responsive to the said electrical stimulation applied to the body portion taking into account the geometry data and the impedance data; and determine a dosage of electrical stimulation impinging on the target treatment region from the electric field data.

It may be that the data processing apparatus is further configured to adjust the electrical signals applied between the electrode and the said pairing electrode to thereby adjust the electrical stimulation applied to the body portion responsive to the determined dosage of electrical stimulation impinging on the target treatment region (e.g. to increase or reduce the electrical stimulation applied to the target treatment region).

It may be that the data processing apparatus is further configured to receive an estimate of an electrical current shunted across the skin interface between the said electrode and the pairing electrode, the data processing apparatus being further configured to determine the dosage of electrical stimulation impinging on the target treatment region from the said determined electric field data taking into account the said estimate of the said electrical current shunted across the skin interface.

It may be that data processing apparatus is configured to mathematically model the electric field through the body portion responsive to the said electrical stimulation by mathematically modelling the quasi-static conduction (QSC) approximation to Maxwell's equations. It may be that the data processing apparatus is configured to mathematically model the electric field through the body portion responsive to the said electrical stimulation by solving the forward problem (i.e. the computation of the electric field distribution in the body portion (e.g. head) resulting from the application of currents to the skin interface (e.g. the scalp)) of the quasi-static conduction (QSC) approximation to Maxwell's equations. It may be that the boundary conditions for the forward problem comprise any one or more (or each) of the following: measured voltages and/or currents at each of the electrodes; any known voltages and currents determined during measurement of the current shunted across the surface of the skin interface; an assumption that no current flows from the skin into the surrounding air; and an assumption that no current disperses from the head and into the neck.

It may be that the electric field data is representative of an electric field through each of a plurality of voxels (i.e. discrete volumes) of within the body portion.

It may be that the data processing apparatus is further configured to determine a dosage of electrical stimulation (e.g. applied by transcranial stimulation) impinging on the target treatment region by volume integration of the determined electric field through the target treatment region (e.g. the sum of the determined electric fields through each of a plurality of voxels representing the target treatment region).

It may be that the data processing apparatus is further configured to determine a total dosage of electrical stimulation impinging on the target treatment region by time integration of a plurality of said determined instantaneous dosages.

A fourteenth aspect of the invention provides a method of estimating a dosage of electrical stimulation impinging on a target treatment region internal to a human body portion (for example the target treatment region may comprise a human brain or a portion of a human brain internal to a head portion), the method comprising: providing geometry data representing a geometry of the human body portion comprising the target treatment region internal to the body portion; providing impedance data indicative of one or more (typically electrical) impedances or resistances (typically data indicative of impedances or resistances of two or more different types of human tissue, such as skin, bone, brain, portions of the brain) of the said body portion; determining an (typically three dimensional) electrical field applied through the body portion, which is responsive to an electrical stimulation applied to the body portion by way of a skin interface of the body portion, taking into account the geometry data and the impedance data; and determining a dosage of electrical stimulation impinging on the target treatment region from the determined electric field data.

It may be that the geometry data comprises a mathematical model and/or image of the body portion.

It may be that the geometry data represents a three dimensional geometry of the human body portion.

It may be that the impedance data is indicative of (typically electrical) impedances or resistances of two or more different types of human tissue (such as skin, bone, brain, portions of the brain) of the said body portion.

It may be that the impedance data comprises data indicative of an impedance or resistance of a first type of human tissue external to the body portion and data indicative of an impedance or resistance of a second type of human tissue internal to the body portion.

It may be that the method further comprises: determining an electrical field applied through the body portion responsive to an electrical stimulation applied to the body portion taking into account the geometry data and the impedance data by using the said geometry data and the impedance data to mathematically model (typically using Maxwell's equations) the electric field applied through the body portion responsive to the said electrical stimulation (e.g. as a function of position).

It may be that the method further comprises determining a dosage of electrical stimulation impinging on the target treatment region from the determined electric field data using predetermined data indicative of the position of the target treatment region within the body portion.

It may be that the method further comprises: providing electrical signals between an electrode and a pairing electrode to thereby apply electrical stimulation to the body portion by way of the skin interface; determining electric field data representing the electrical field applied through the body portion responsive to the electrical stimulation applied to the body portion by the electrodes taking into account the geometry data and the impedance data; and determining a dosage of electrical stimulation impinging on the target treatment region from the electric field data.

It may be that the method further comprises adjusting the electrical signals applied between the electrode and the pairing electrode to thereby adjust the electrical stimulation applied to the body portion responsive to the determined dosage of electrical stimulation impinging on the target treatment region.

It may be that the method further comprises receiving an estimate of an electrical current shunted across the skin interface between the said electrode and the pairing electrode; and determining the dosage of electrical stimulation impinging on the target treatment region from the said determined electric field data taking into account the said estimate of the electrical current shunted across the skin interface.

It may be that the method further comprises determining a dosage of electrical stimulation (e.g. applied by transcranial stimulation) impinging on the target treatment region by volume integration of the electric field data relating to the target treatment region (e.g. the determined electric field through each of a plurality of voxels representing the target treatment region).

It may be that the method further comprises determining a total dosage of electrical stimulation impinging on the target treatment region by time integration of a plurality of said determined instantaneous dosages.

A fifteenth aspect of the invention provides an electrode module for non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the electrode module having: an (first) end for defining (or configured to define) an electrolyte application region (typically comprising electrolyte in use) between the electrode module and the skin interface; and a plurality of (typically individual) electrodes which are electrically couplable or electrically coupled to the skin interface by way of an electrolyte in the said electrolyte application region, the electrodes being spaced apart from each other, wherein the said electrodes are configured so that electrical signals (e.g. voltage and/or current) to each of the said electrodes can be adjusted individually (and typically selectively).

It will be understood that typically, in use, electrolyte is provided in the electrolyte application region.

Typically the electrodes of the (first) electrode module are configured so that the electrical potential of each of the said electrodes can be adjusted individually (typically selectively, typically independently of the potentials of the other electrodes). Typically the electrodes of the (first) electrode module are configured so that the electrical current flowing through each of the said electrodes can be adjusted individually (and typically selectively).

Typically there is no fixed electrical coupling between the electrodes of the electrode module so that electrical signals (e.g. voltage and/or current) to each of the said electrodes can be adjusted individually (and typically selectively).

Typically the electrode module further comprises one or more shunt measurement conductors. It may be that one or more or each of the shunt measurement conductors are provided between the electrodes of the electrode module and an edge of the said (first) end of the electrode module.

Typically the electrode module is provided as part of an electrode apparatus further comprising a controller configured to individually (and typically selectively) adjust electrical signals applied to each of the said plurality of electrodes. Typically the controller is further configured to individually (and typically selectively) measure electrical signals from each of the said plurality of electrodes.

A sixteenth aspect of the invention provides an electrode module for non-invasively applying (or configured to non-invasively apply) (e.g. transcranial) electrical stimulation to a body portion (typically to a target treatment region of a body portion internal to the body portion, such as a brain or a portion of the brain) of a human subject by way of a skin interface (e.g. a skin interface of the subject's scalp), the electrode module having: an (first) end for defining (or configured to define) an electrolyte application region (typically comprising electrolyte in use) between the electrode module and the skin interface; one or more (typically individual) electrodes which are electrically couplable or electrically coupled to the skin interface by way of an electrolyte in the said electrolyte application region; and one or more shunt measurement conductors provided between the said electrode(s) and an edge of the said (first) end of the electrode module.

It will be understood that typically, in use, electrolyte is provided in the electrolyte application region.

The invention also extends to electrode apparatus for treatment of neurological disorders, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of psychiatric disorders, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of depression, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of Parkinson's disease, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of dystonia, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of obsessive compulsive disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of epilepsy, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of migraine, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of essential tremor, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of a sleep disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of pain, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of mood disorders, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for influencing mood of a subject, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for improving cognition, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of attention deficit disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of addiction, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of alcohol addiction, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of Alzheimer's disease, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of anxiety, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of aphasia, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of autism, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of auditory disorders, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of bipolar disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of cerebral palsy, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of dysphagia, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of fibromyalgia, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of hemiparesis, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of impairment, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of injury, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of multiple sclerosis, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of nicotine addiction, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of obesity, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of post traumatic stress disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of schizophrenia, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of stroke, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of tinnitus, the electrode apparatus comprising the electrode apparatus according to any of the first, fifth, seventh, ninth and eleventh aspects of the invention or the electrode module according to the fifteenth or sixteenth aspects of the invention.

The invention also extends to electrode apparatus for treatment of Tourette's syndrome, the electrode apparatus comprising the electrode apparatus according to any of the first, fifth, seventh, ninth and eleventh aspects of the invention or the electrode module according to the fifteenth or sixteenth aspects of the invention.

The invention also extends to electricity for treatment of neurological disorders, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of psychiatric disorders, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of depression, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of Parkinson's disease, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of dystonia, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of obsessive compulsive disorder, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of epilepsy, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of migraine, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of essential tremor, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of a sleep disorder (e.g. insomnia), the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of pain, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of mood disorders, the electricity being applied by the electrode apparatus according to any of the first, fifth, seventh, ninth and eleventh aspects of the invention or the electrode module according to the fifteenth or sixteenth aspects of the invention.

The invention also extends to electricity to influence mood of a subject, the electricity being applied by the electrode apparatus according to any of the first, fifth, seventh, ninth and eleventh aspects of the invention or the electrode module according to the fifteenth or sixteenth aspects of the invention.

The invention also extends to electricity for improving cognition, the electricity being applied by the electrode apparatus according to any of the first, fifth, seventh, ninth and eleventh aspects of the invention or the electrode module according to the fifteenth or sixteenth aspects of the invention.

The invention also extends to electrode apparatus for treatment of attention deficit disorder, the electrode apparatus comprising the electrode apparatus according to any of the first, fifth, seventh, ninth and eleventh aspects of the invention or the electrode module according to the fifteenth or sixteenth aspects of the invention.

The invention also extends to electricity for treatment of addiction, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of alcohol addiction, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of Alzheimer's disease, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of anxiety, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of aphasia, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of autism, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of auditory disorders, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of bipolar disorder, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of cerebral palsy, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of dysphagia, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of fibromyalgia, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of hemiparesis, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of impairment, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of injury, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of multiple sclerosis, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of nicotine addiction, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of obesity, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of post traumatic stress disorder, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of schizophrenia, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of stroke, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of tinnitus, the electricity being applied by the electrode apparatus according to any of the first, fifth, seventh, ninth and eleventh aspects of the invention or the electrode module according to the fifteenth or sixteenth aspects of the invention.

The invention also extends to electricity for treatment of Tourette's syndrome, the electricity being applied by the electrode apparatus according to any of the first, fifth, seventh, ninth and eleventh aspects of the invention or the electrode module according to the fifteenth or sixteenth aspects of the invention.

Any of the methods according to the second, third, sixth, eighth, tenth, twelfth and fourteenth aspects of the invention can be used in the treatment of neurological disorders.

Any of the methods according to the second, third, sixth, eighth, tenth, twelfth and fourteenth aspects of the invention can be used in the treatment of psychiatric disorders.

Any of the methods according to the second, third, sixth, eighth, tenth, twelfth and fourteenth aspects of the invention can be used in the treatment of depression.

Any of the methods according to the second, third, sixth, eighth, tenth, twelfth and fourteenth aspects of the invention can be used in the treatment of Parkinson's disease.

Any of the methods according to the second, third, sixth, eighth, tenth, twelfth and fourteenth aspects of the invention can be used in the treatment of dystonia.

Any of the methods according to the second, third, sixth, eighth, tenth, twelfth and fourteenth aspects of the invention can be used in the treatment of obsessive compulsive disorder.

Any of the methods according to the second, third, sixth, eighth, tenth, twelfth and fourteenth aspects of the invention can be used in the treatment of epilepsy.

Any of the methods according to the second, third, sixth, eighth, tenth, twelfth and fourteenth aspects of the invention can be used in the treatment of migraine.

Any of the methods according to the second, third, sixth, eighth, tenth, twelfth and fourteenth aspects of the invention can be used in the treatment of essential tremor.

Any of the methods according to the second, third, sixth, eighth, tenth, twelfth and fourteenth aspects of the invention can be used in the treatment of a sleep disorder.

Any of the methods according to the second, third, sixth, eighth, tenth, twelfth and fourteenth aspects of the invention can be used in the treatment of pain.

Any of the methods according to the second, third, sixth, eighth, tenth, twelfth and fourteenth aspects of the invention can be used in the treatment of mood disorders.

Any of the methods according to the second, third, sixth, eighth, tenth, twelfth and fourteenth aspects of the invention can be used to influence mood of a subject.

Any of the methods according to the second, third, sixth, eighth, tenth, twelfth and fourteenth aspects of the invention can be used to improve cognition.

Any of the methods according to the second, third, sixth, eighth, tenth, twelfth and fourteenth aspects of the invention can be used in the treatment of attention deficit disorder.

Any of the methods according to the second, third, sixth, eighth, tenth, twelfth and fourteenth aspects of the invention can be used in the treatment of addiction.

Any of the methods according to the second, third, sixth, eighth, tenth, twelfth and fourteenth aspects of the invention can be used in the treatment of alcohol addiction.

Any of the methods according to the second, third, sixth, eighth, tenth, twelfth and fourteenth aspects of the invention can be used in the treatment of Alzheimer's disease.

Any of the methods according to the second, third, sixth, eighth, tenth, twelfth and fourteenth aspects of the invention can be used in the treatment of anxiety.

Any of the methods according to the second, third, sixth, eighth, tenth, twelfth and fourteenth aspects of the invention can be used in the treatment of aphasia.

Any of the methods according to the second, third, sixth, eighth, tenth, twelfth and fourteenth aspects of the invention can be used in the treatment of autism.

Any of the methods according to the second, third, sixth, eighth, tenth, twelfth and fourteenth aspects of the invention can be used in the treatment of auditory disorders.

Any of the methods according to the second, third, sixth, eighth, tenth, twelfth and fourteenth aspects of the invention can be used in the treatment of bipolar disorder.

Any of the methods according to the second, third, sixth, eighth, tenth, twelfth and fourteenth aspects of the invention can be used in the treatment of cerebral palsy.

Any of the methods according to the second, third, sixth, eighth, tenth, twelfth and fourteenth aspects of the invention can be used in the treatment of dysphagia.

Any of the methods according to the second, third, sixth, eighth, tenth, twelfth and fourteenth aspects of the invention can be used in the treatment of fibromyalgia.

Any of the methods according to the second, third, sixth, eighth, tenth, twelfth and fourteenth aspects of the invention can be used in the treatment of hemiparesis.

Any of the methods according to the second, third, sixth, eighth, tenth, twelfth and fourteenth aspects of the invention can be used in the treatment of impairment.

Any of the methods according to the second, third, sixth, eighth, tenth, twelfth and fourteenth aspects of the invention can be used in the treatment of injury.

Any of the methods according to the second, third, sixth, eighth, tenth, twelfth and fourteenth aspects of the invention can be used in the treatment of multiple sclerosis.

Any of the methods according to the second, third, sixth, eighth, tenth, twelfth and fourteenth aspects of the invention can be used in the treatment of nicotine addiction.

Any of the methods according to the second, third, sixth, eighth, tenth, twelfth and fourteenth aspects of the invention can be used in the treatment of obesity.

Any of the methods according to the second, third, sixth, eighth, tenth, twelfth and fourteenth aspects of the invention can be used in the treatment of post traumatic stress disorder.

Any of the methods according to the second, third, sixth, eighth, tenth, twelfth and fourteenth aspects of the invention can be used in the treatment of schizophrenia.

Any of the methods according to the second, third, sixth, eighth, tenth, twelfth and fourteenth aspects of the invention can be used in the treatment of stroke.

Any of the methods according to the second, third, sixth, eighth, tenth, twelfth and fourteenth aspects of the invention can be used in the treatment of tinnitus.

Any of the methods according to the second, third, sixth, eighth, tenth, twelfth and fourteenth aspects of the invention can be used in the treatment of Tourette's syndrome.

The preferred and optional features of each aspect of the invention disclosed herein are preferred and optional features of each other aspect of the invention to which they are applicable. For the avoidance of doubt, the preferred and optional features of each aspect of the invention are also preferred and optional features of all of the other aspects of the invention, where applicable.

It will be understood that each aspect of the invention disclosed herein is compatible with each of the other aspects of the invention disclosed herein. Accordingly, the invention extends to any combination of any of the aspects of the invention disclosed herein.

### DESCRIPTION OF THE DRAWINGS

An example embodiment of the present invention will now be illustrated with reference to the following Figures in which:

FIGS. 1A and 1B are perspective views of the prior-art in Transcranial Electrical Stimulation (TES) showing electrode assemblies using alternatively sponge and saline (FIG. 1A) and conductive rubber and electro-paste (FIG. 1B) electrolytes;

FIGS. 2A to 2C show different electrodes used in Electroencephalography (EEG), including: a wet-electrode for resistive sensing (FIG. 2A); dry-electrodes for capacitive sensing (FIG. 2B); and an electrode with active amplification (FIG. 2C).

FIG. 3 illustrates the typical process of application of electro-gel onto the scalp through the holes of a typical rubber EEG electrode cap and before electrode placement;

FIG. 4 shows a perspective view of the prior art in brain Electrical Impedance Tomography (EIT);

FIG. 5 is a representation of a mathematical model of a human head, which includes assumptions of representative geometry and electrical conductivity and impedance of the various tissue layers;

FIG. 6A illustrates transcranial electrical stimulation being applied to a human subject under the supervision of a clinician or supervisor, the subject wearing a headset to position two electrode modules for applying electrical stimulation to a target treatment region of the brain of the human subject;

FIG. 6B is a close-up view of one of the electrode modules of FIG. 6A;

FIG. 7A is a perspective schematic view of an electrode module comprising an electrode array for applying electrical stimulation to a human subject by way of an electrode-to-skin interface and various control electronics;

FIG. 7B is a magnified view showing multiple electrodes, electrolyte passages and impedance measuring vectors;

FIGS. 8A-8D show four sectioned, side elevation views showing various alternative electrode arrays and together with the skin interface;

FIG. 9 is a flow diagram of a control algorithm implemented by the control electronics;

FIG. 10 is a flow diagram of an algorithm for characterising the impedance between the electrode modules of FIGS. 7A, 7B;

FIG. 11 is a flow diagram of an algorithm for characterising the impedance in electrolyte application regions defined by the electrode modules of FIGS. 7A, 7B in more detail;

FIGS. 12A and 12B are plan views of alternative undersides of the electrode modules;

FIG. 13 is a flow diagram of an algorithm for calculating a dosage of electrical stimulation impinging on the target treatment region;

FIG. 14 is a partially cut away three quarter view above and to the side of the head of a human subject, showing the position of a target treatment region within the human brain;

FIG. 15A is a cut-away view of a bio-impedance model of a human head to which transcranial electrical stimulation is being applied by the electrode modules of FIGS. 7A, 7B, and a four-conductor method for measuring the current shunted over the skin between the two electrode modules;

FIG. 15B is an equivalent electrical circuit showing two parallel paths between the electrode modules of FIG. 15A which can be used to estimate the current shunted over the skin between the two electrode modules, and thus the current flowing within the cranium;

FIG. 15C is a similar equivalent electrical circuit to FIG. 15B, but with the shunt measurement conductor(s) of the second electrode module omitted;

FIGS. 16A-16D are perspective views of an electrode module of FIGS. 7A, &B having alternative arrangements of shunt measurement conductors provided on its underside (the electrodes are not shown in detail in FIGS. 16A-16D);

FIG. 17 is a flow diagram of an algorithm for calculating the current shunted across the skin between the electrode modules of FIGS. 7A, 7B;

FIG. 18 shows apparatus for detecting side-effects (or one or more pre-ictal or pre-migraine aura states of) the human patient caused by the application of electrical stimulation and a control system for taking corrective action in response thereto;

FIG. 19 is a flow diagram of an algorithm performed by the controller in response to data from the apparatus for detecting side effects of FIG. 18;

FIGS. 20-22 are perspective views of various electrolyte dispensing apparatus for controlling delivery of electrolyte to the electrolyte application region, provided as part of an electrode module of FIGS. 7A, 7B;

FIGS. 23A-23D are perspective views of an electrode module of FIGS. 7A, 7B, each view showing a different electrolyte containment apparatus;

FIGS. 24 and 25 are flow diagrams of a closed-loop algorithm for applying electrolyte to the electrolyte application region of an electrode module of FIGS. 7A, 7B;

FIG. 26 is a flow diagram of an open loop algorithm for applying electrolyte to the electrolyte application region of an electrode module of FIGS. 7A, 7B; and

FIG. 27 is a flow diagram of an algorithm for adjusting stimulation applied to the body portion.

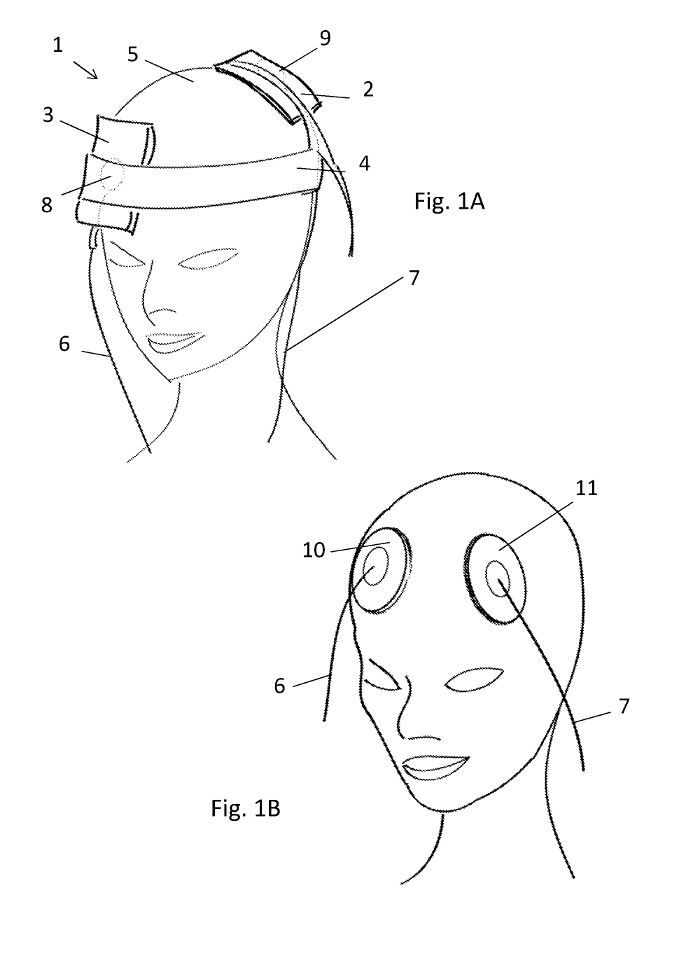

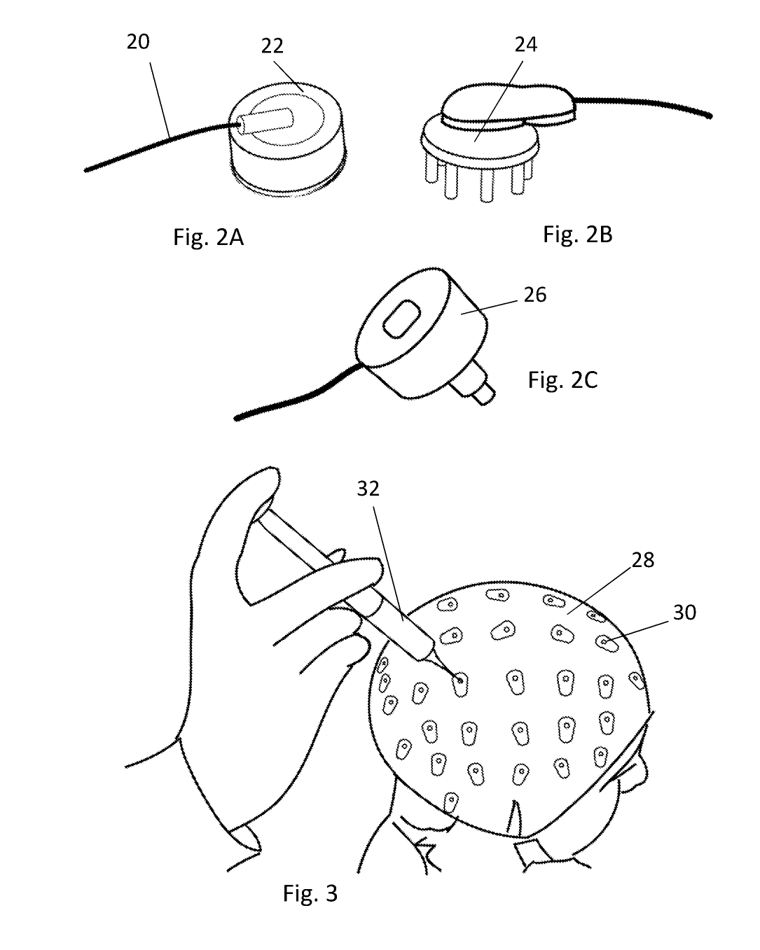

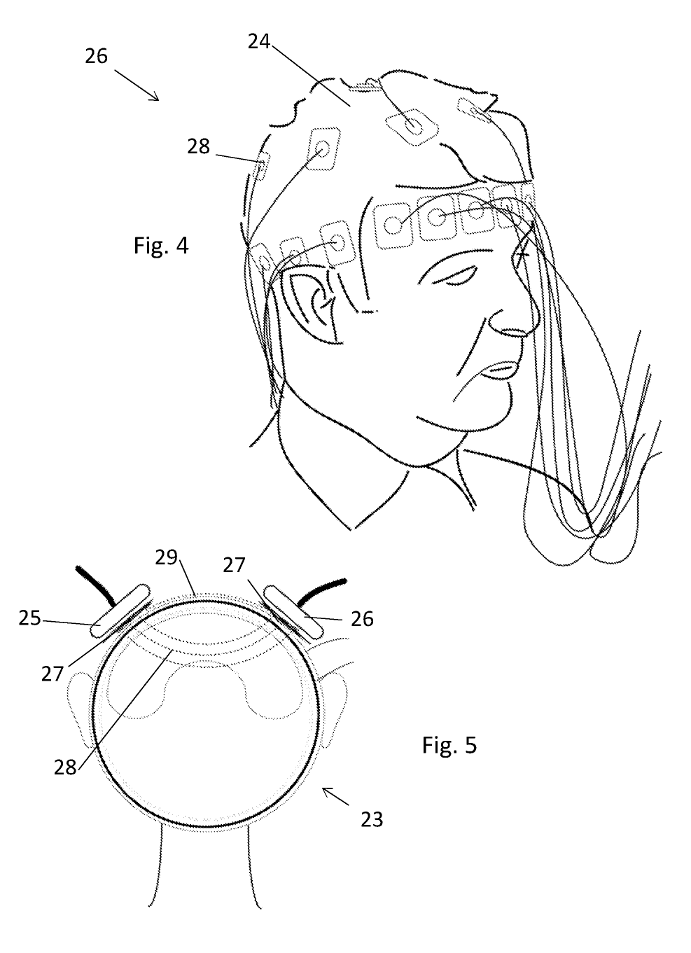

### DETAILED DESCRIPTION OF AN EXAMPLE EMBODIMENT

FIG. 4 illustrates an application of electrical impedance tomography to determine an image 23 of the head 24 of a subject 26 using a plurality of point electrodes 28 attached to the head 24. Typically, a plurality of 2D images are determined from measurements made using the electrodes 28, and the 2D images are combined for form a ‘real’ 3D geometric brain/head model of the impedance of the head, where the greyscale value represents the reconstructed impedance value from the measured data. FIG. 5 illustrates a simplified conceptual mathematical model 23 of the impedance of the human head for transcranial electrical stimulation, the model 23 comprising several tissue/impedance layers inside the head, two monolithic electrodes 25, 26, electrolyte 27 and electrical current flow 28, 29 through the head and shunted across the scalp.

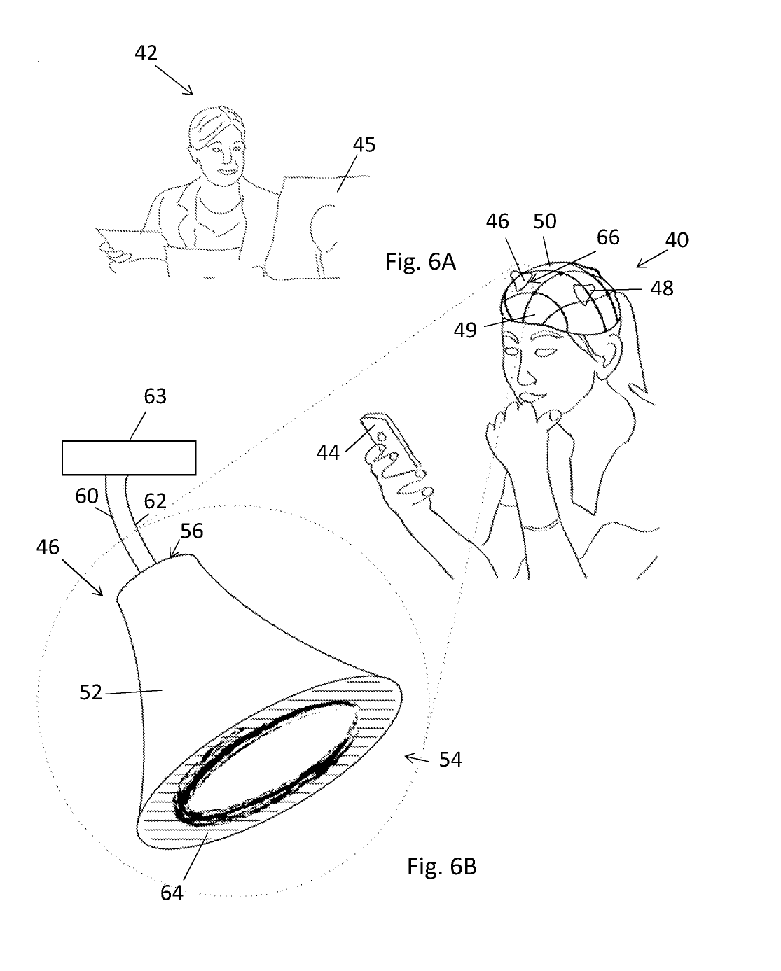

FIG. 6A illustrates a transcranial electrical stimulation session of a human subject 40, typically under the control of a clinician 42 (although it may be that the stimulation session is performed by the subject without the clinician being present, in which case the clinician sets up the session in advance but the subject 40 may start the session at his/her convenience). The human subject 40 and clinician 42 both use (typically internet-connected) electronic communication devices 44, 45 (which may be portable or mobile electronic communications devices such as mobile smartphones, tablets, laptop computers and so on) to control the stimulation session. These devices 44, 45 are used to: set-up parameters for the stimulation session; control the stimulation session while it is in progress; and/or to report feedback before, during and after the session.

Alternating current (AC) electrical transcranial stimulation is non-invasively applied to a target treatment region (e.g. left dorsolateral prefrontal cortex (DLPFC) of the brain for treatment of depression) internal to the head of the subject 40 by applying electrical signals across or between electrodes of two identical electrode modules 46, 48 which are spaced apart from each other (on opposite sides of the subject's head) and retainably mounted to a skin interface 49 of the subject's head by way of a cap 50. Any other alternative means (such as a head band or headset) may be used to hold the electrode modules 46, 48 in place or the electrodes may be held in place by the viscosity of an electrolyte provided between the modules 46, 48 and the head. As electrode modules 46, 48 are identical to each other, only electrode module 46 will be described in detail for brevity.

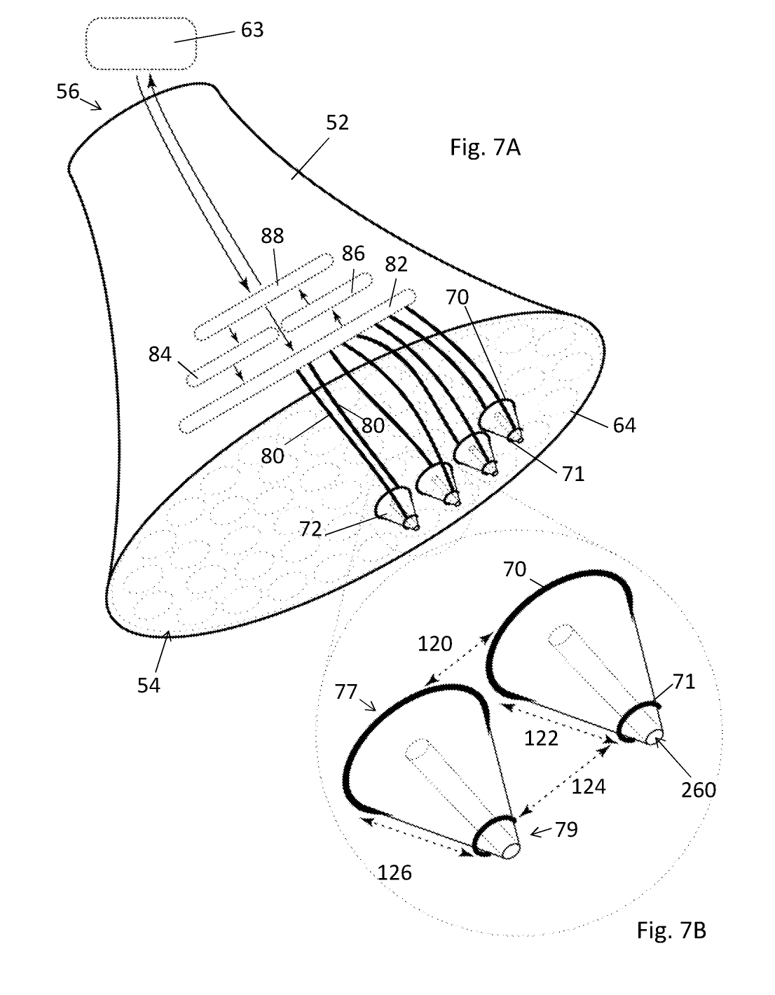

As shown in more detail in FIG. 6B, the electrode module 46 comprises a bell-shaped electrode housing 52 (although it is noted that the electrode housing 52 could be any suitable alternative shape) having opposing first and second ends 54, 56 between which the housing 52 extends, the first end 54 having a greater than diameter than the second end 56, the housing 52 increasing in diameter as it extends from the second end 56 to the first end 54. Two conductors 60, 62 extend from the second end 56 of the electrode module 46, the conductors 60, 62 being connected to a control module 63, at least part of which is external to the housing 52 in this illustrated embodiment (although this is not necessarily the case) and being configured to deliver electrical signals to a plurality of electrodes (not shown in FIG. 6A or 6B—see FIGS. 7A, 7B described below) of the electrode module 46 by way of the conductors 60, 62. The first end 54 of the electrode housing has a surface 64 which mirrors the curvature of the head and which defines an electrolyte application region 66 between the electrode module 46 and the skin interface 49.

FIG. 7A is a schematic perspective view of the electrode module 46 showing a three dimensional array of electrodes at the first end 54 of the electrode module 46. A plurality of (typically non-electrically-conductive) frusto-conical axial members 72 are provided which extend from (and optionally through) the surface 64 of the first end 54 of the electrode housing 52 at positions distributed across the surface 64, and two individual annular electrodes 70, 71 are provided on external surfaces of each of the axial members 72, the annular electrodes 70, 71 on each axial member 72 being axially offset from each other in a direction parallel to the longitudinal axis of the axial member 72 (and in a direction parallel to the line of shortest distance between the first and second ends 54, 56 of the electrode housing 52). There is no fixed electrical coupling between the electrodes of the electrode module (albeit the electrodes are typically brought into electrical communication with each other by electrolyte provided in the electrolyte application region, and albeit the same electrical signals may under some circumstances be applied to each of the electrodes of the electrode modules 46, 48). Accordingly, electrical signals applied to each of the electrodes of the electrode modules 46, 48 can be individually and selectively adjusted. Indeed the electrical potentials of each of the electrodes can be set independently of the electrical potentials of the other electrodes.

The electrode module 46 may be manufactured by 3D printing or injection moulding (for example). In this case, the axial members 72 are typically integrally formed with the housing 52. The electrodes 70, 71 may be retainably mounted in slots provided on the external surfaces of the axial members 72. It may be that the electrodes 70, 71 do not extend all the way around the axial members 72 (e.g. similar to a circlip). Additionally or alternatively, the electrodes may be held in place by conductors 80 (see below). It may be that the electrodes 70, 71 are glued or melted (assuming the axial members are thermoplastic) into the external surfaces of the axial members 72. Alternatively, the end 54 of the electrode module 46 may be manufactured using printed circuit board manufacturing techniques, in which case it may be that the electrodes 70, 71 are created as conductive tracks in the PCB and electrolyte delivered to the electrolyte application region through holes in the PCB (rather than through the axial members 72—see below).

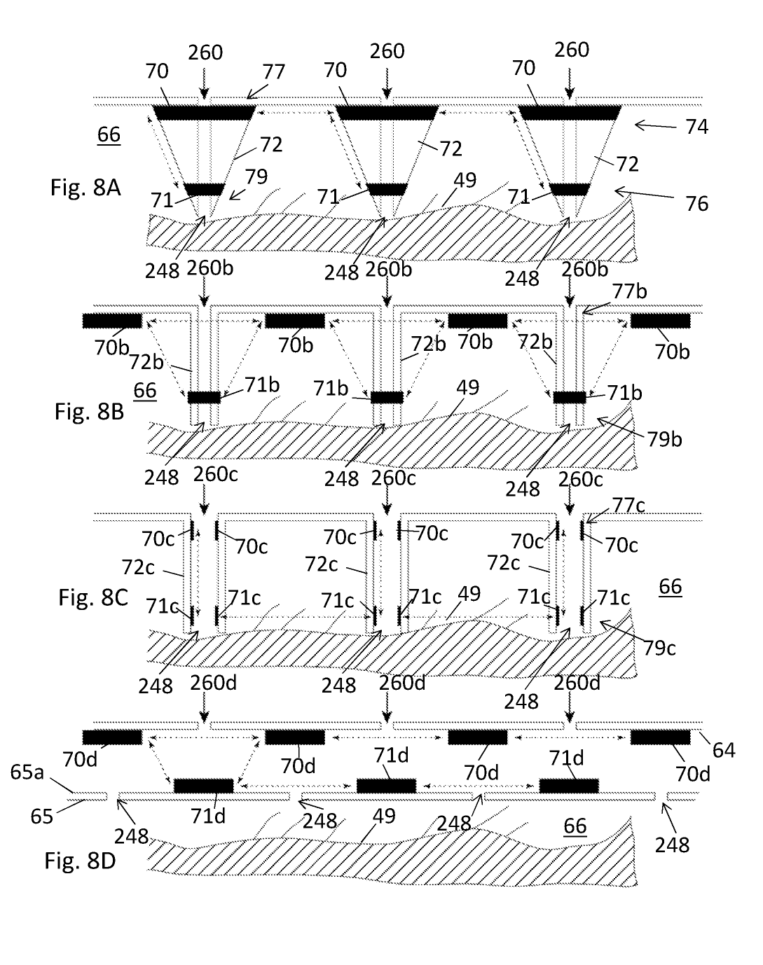

FIG. 8A is a side sectional view showing three of the axial members 72 and six of the electrodes 70, 71 of the electrode module 46 of FIG. 7A installed on the skin interface 49. As can be seen from FIGS. 7A, 7B and 8A, the frusto-conical axial members 72 have a greater diameter, proximal end 77 and a lower diameter, distal end 79 and the electrodes 70 are retainably mounted on the greater diameter ends 77 of the axial members 72, while the electrodes 71 are retainably mounted to the axial members 72 at positions closer to the lower diameter ends 79 of the axial members than to the greater diameter ends 77. Some of the electrodes and axial members are omitted from FIGS. 7-8 for clarity.

The electrodes are thus arranged into first and second two dimensional arrays 74, 76, first two dimensional array 74 comprising the electrodes 70 and the second two dimensional array 76 comprising the electrodes 71. Within each of the two dimensional arrays 74, 76, the electrodes are spaced from each other in a plane parallel to the plane of the surface 64 of the first end 54 of the electrode housing. The two dimensional arrays 74, 76 are axially offset from each other in a direction parallel to the line of shortest distance between the first and second ends 54, 56 of the electrode housing 52. The electrodes 70, 71 of the electrode module 46 are in electrical communication with each other and the skin interface 49 by way of electrolyte provided in the electrolyte application region between the surface 64 of the module 46 and the skin interface 49. Each electrode 70, 71 may be from a fraction of a millimetre to several millimetres in diameter (it being understood that the electrodes 70 are all typically the same size as each other, and the electrodes 71 are all typically the same size as each other).

As shown in FIGS. 8B-8D, the electrodes do not need to be annular, nor do they need to be mounted on axial members 72 by way of annuluses. In FIG. 8B, electrodes 70b are provided in place of electrodes 70, the electrodes 70b being mounted to the surface 64 of the first end 54 of the electrode module 46 which defines the electrolyte application region 66 between the electrode module 46 and the skin interface 49. Cylindrical axial members 72b, having proximal ends 77b which extend from the surface 64 of the first end 54 of the electrode module 46 at positions between adjacent electrodes 70b, are provided instead of the frusto-conical axial members 72 of FIG. 8A, and alternative annular electrodes 71b are provided instead of the electrodes 71 of FIG. 8A, the annular electrodes 71b being mounted on external surfaces of the respective axial members 72b near distal ends 79b thereof by way of their annuluses. Pairs of electrodes 70b and 71b are offset from each other in a direction parallel to the longitudinal axis of the axial member 72b on which they are provided.

In FIG. 8C, alternative cylindrical axial members 72c which have proximal ends 77c extending from the surface 64 of the first end 54 of the electrode module 46, are provided instead of the frusto-conical axial members 72 of FIG. 8A while alternative electrodes 70c are provided (i.e. alternatives to electrodes 70), each electrode 70c being mounted to an internal surface of a cylindrical axial member 72c at an axial position closer to the proximal end 77c of the cylindrical axial member 72c than to the distal end 79c thereof. Alternative electrodes 71c are also provided (i.e. alternatives to electrodes 71), each being mounted to an internal surface of a cylindrical axial member 72c at an axial position closer to the distal end 79c of the cylindrical axial member 72c than to the proximal end 77c thereof.

In FIG. 8D, electrodes 70d are provided instead of the electrodes 70 of FIG. 8A, the electrodes 70d being mounted to the surface 64 of the first end 54 of the electrode module 46 which defines the electrolyte application region between the electrode module 46 and the skin interface 49. In addition, electrodes 71d are provided instead of the electrodes 71 of FIG. 8A, the electrodes 71d being mounted to an internal surface 65a of a plate 65 mechanically coupled to the surface 64 and being parallel to the plane of surface 64, but axially offset therefrom towards the skin interface 49 in a direction parallel to the line of shortest distance between the first and second ends 54, 56 of the electrode housing 52. The electrodes 70d, 71d are offset from each other in directions parallel and perpendicular to the line of shortest distance between the first and second ends 54, 56 of the electrode housing 52. In the case of FIG. 8, the electrolyte application region extends between the surfaces 64, 65a and between the plate 65 and the skin interface 49.

Unless otherwise stated, the following description assumes that the electrode arrangement of FIGS. 7A, 7B and FIG. 8A are employed, but it will be appreciated that any of the alternative arrangements of FIGS. 8B-D could be used instead.

As shown in FIG. 7A, each of the electrodes 70, 71 are connected through respective conductors 80 (typically at least one per electrode) to a switch matrix 82, which controls a selective connection between each individual electrode 70, 71 of the electrode module 46 and a multi-channel signal generator 84. This allows the electrical potential of each electrode to be individually selected. The switch matrix 82 also controls a selective connection between each individual electrode 70, 71 of the electrode module 46 and a multi-channel impedance calculator 86. The signal generator 84 and impedance calculator 86 are provided in communication with a modelling module 88 which is itself in communication with the control module 63. As described in detail below, the switch matrix 82, multi-channel signal generator 84, multi-channel impedance calculator 86, modelling module 88 and control module 63 of the first and second electrode modules 46, 48 together function as a controller for controlling electrical stimulation applied to the targeted treatment region by the electrode modules 46, 48.

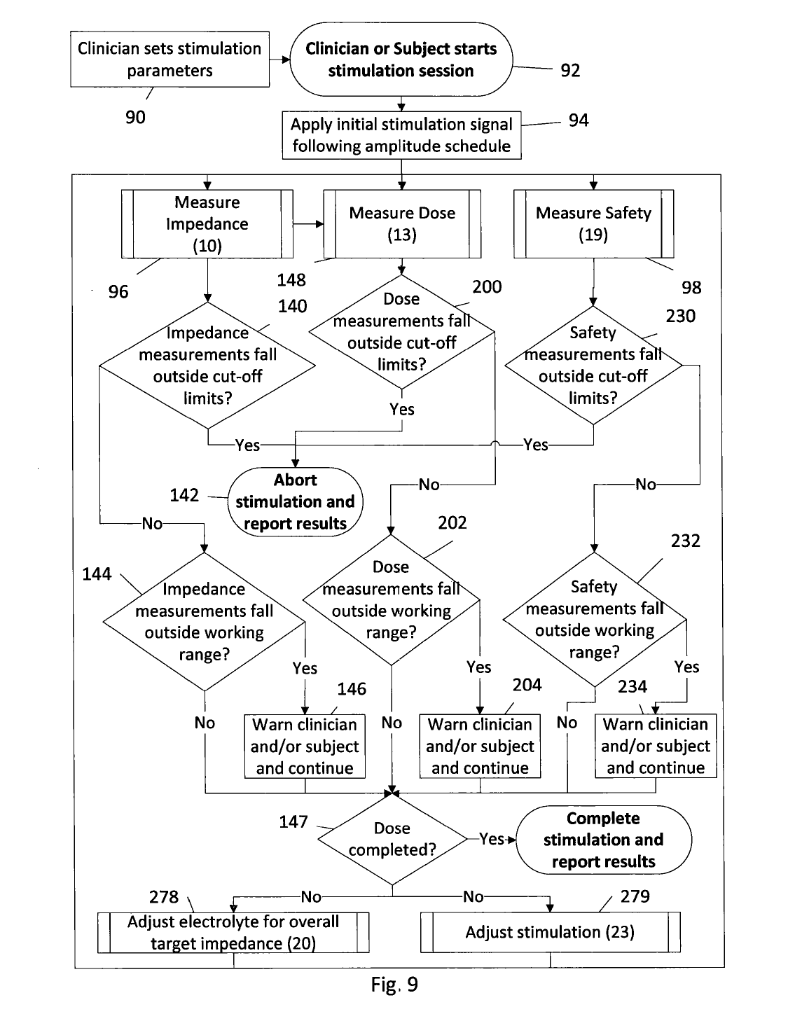

FIG. 9 is a flow diagram illustrating a control algorithm performed by the control module 63. In a preliminary step 90, the clinician 42 sets a number of stimulation parameters, including some or all of the following: an electrical stimulation dosage to be applied to the subject 40, which may be provided in the form of a current amplitude schedule to be applied to the skin interface by the electrode modules 46, 48 over a stimulation session and/or a target instantaneous and/or total dosage to be applied to the subject 40; safety limits, which may be provided in the form of acceptable ranges of impedance between one or more electrodes of the electrode modules 46, 48 and the skin interface 49 (which ranges may be defined with reference to one or more threshold values or limits), acceptable instantaneous and/or total dosage ranges (which may be defined with reference to one or more threshold values or limits) and/or acceptable ranges of values of one or more physiological stress indicators indicative of a physiological stress of the subject 40 or of a function of two or more said physiological stress indicators; geometry data representing a geometry (e.g. size and/or shape) of the head of the subject (such as that shown in FIG. 5), which may be provided in the form of standard geometry data which is not specific to the human subject, typically together with physical measurements of the subject's head (which may be provided by way of an image of the subject's head produced by measurement or imaging (e.g. EIT (see FIGS. 4, 5), 3D scanning, CAT scanning, MRI imaging or similar); predetermined data indicative of the position of the target treatment region within the head; subject's (e.g. manually, e.g. verbally) reported physiological stress indicators, such as skin sensitivity or anxiety levels (this is typically an ongoing input into the algorithm); objective functions and/or target accuracy levels of modelling to be performed by the modelling module 88; impedance data indicative of (typically electrical) impedances or resistances of one or more type (typically two or more different types) of tissue of the human head and/or permittivity properties or similar. Some of these parameters may alternatively be pre-loaded into the control module 63 (e.g. during manufacture). Typically the impedance data comprises typical impedances (e.g. as a function of frequency) of the tissue types external to the head (e.g. skin, hair) as well as internal to the head (e.g. bone, (different types of) brain tissue). The determination of these parameters may be assisted by measurements from previous stimulation sessions and/or from measurements taken before the stimulation is started for this session.

In a next step 92, the clinician 42 or subject 40 starts the stimulation session. Next, in step 94, initial alternating current (AC) electrical stimulation signals are applied to the electrodes 70, 71 in accordance with the current amplitude schedule set by the clinician.

A problem observed in the application of transcranial stimulation to a subject using known electrode apparatus (which typically provides monolithic electrodes with large contact surface areas, such as those shown in FIG. 5) is that the spatial distribution of electrical current between electrodes and the skin interface is subject to significant variation, particularly because of the leakage and/or drying out of electrolyte between the electrode contact area and the skin interface. These variations cause local increases in the current density at the skin interface, which can cause irritation and pain to the subject. These variations cannot typically be detected until the subject complains of irritation or pain, and so significant manual effort is required to monitor the region between the electrodes and the skin interface to ensure that there is sufficient electrolyte. An aim of the present invention is to (automatically) determine the spatial distribution of current within the electrolyte application regions between the electrode modules 46, 48 and the skin interface, in order that local variations in current density can be identified prior to the subject experiencing irritation or pain, and to permit corrective action (e.g. dispensing additional electrolyte or adjusting stimulation applied to the electrodes) to be taken to prevent the said irritation or pain from occurring.

Accordingly, in two parallel next steps 96, 98, the algorithm: generates a three dimensional, dynamically updated mathematical model of the impedance between the electrode modules 46, 48 and the skin interface 49, and within the head, as a function of position; and measures one or more physiological stress indicators to determine whether the subject 40 is experiencing or is likely to experience a form of physiological stress responsive to the stimulation being applied to the head by the electrode modules 46, 48. These parallel steps 96, 98 are now explained in more detail in turn.

By virtue of the fact that the electrode modules 46, 48 comprise a number of individual electrodes (which are typically not electrically coupled to each other within the electrode modules themselves), a detailed model of the impedance between the electrode modules 46, 48 and the skin interface can be determined which indicates local variations in the impedance within the electrolyte application regions. Local variations in the impedance within the electrolyte application regions are typically indicative of local variations in the current density within the electrolyte application region. This is because both the impedance and the current density are responsive to whether there is sufficient electrolyte between the electrodes and the skin interface, and between the electrodes themselves, within the electrolyte application region. If there is sufficient electrolyte between the electrode modules 46, 48 and the skin interface 49, there should not be much variation between the calculated impedances in respect of each pair of electrodes, other than variations caused by varying distances between the electrodes of the pairs. However, if there is insufficient electrolyte between one or more pairs of electrodes, the impedance between the electrodes of those pairs will be greater. By measuring the impedance between electrodes which are spaced from each other across the electrolyte application region it can be determined whether (and where) there are any dry patches (i.e. patches with no or little electrolyte) between them (e.g. if the impedance is high). It can also be determined whether there are dry patches between the electrodes and the skin interface. Accordingly, by building up a model of the impedance between the electrode modules 46, 48 and the skin interface 49, the spatial distribution of current within the electrolyte application regions can be determined.

Although the following description provides a mathematical model (e.g. 2D mathematical surface or 3D mathematical volume) representing the impedance variations within the electrolyte application region, it may be that the measured impedance variations within the electrolyte application region are stored with reference to any suitable alternative framework.

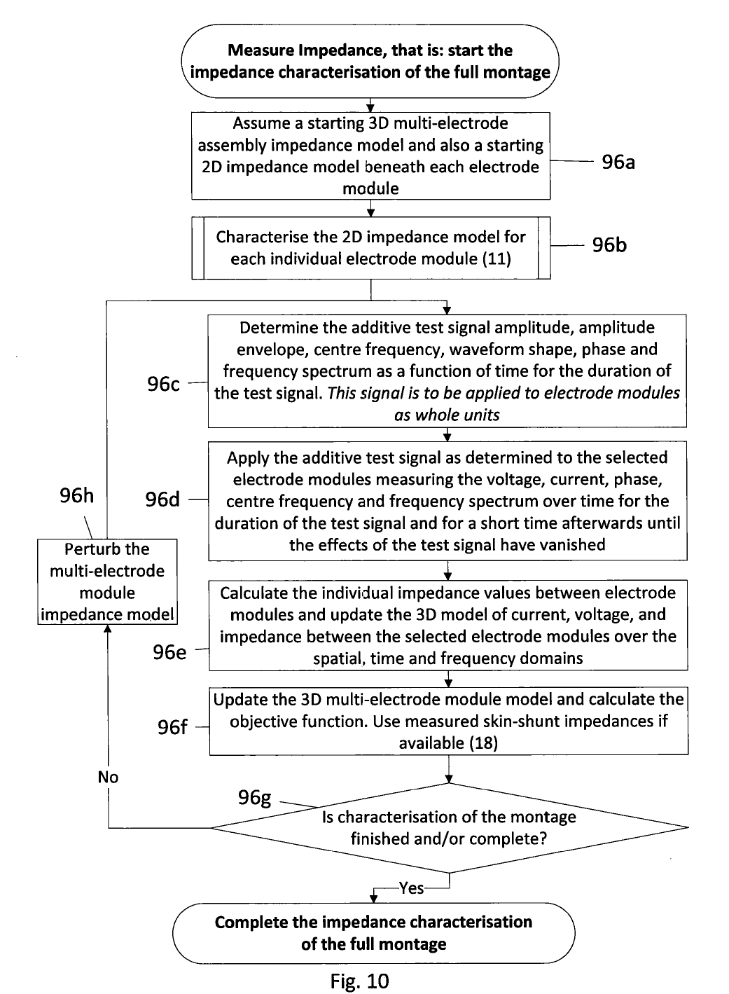

As illustrated by the flow diagram of FIG. 10, in order to generate a three dimensional model of the impedance as a function of position within the electrolyte application regions, and within the head, an initial three dimensional impedance model as a function of position is generated in step 96a using the geometry data and the impedance data provided by the clinician. The model may be an analytical model, such as the following (“4-shell model”) where the geometry data assumes a simplified geometry of the head comprising four concentric spheres including a first sphere representing the cortex, a second sphere representing the intermediate layers (typically including the cerebral spinal fluid) between the cortex and the skull, a third sphere representing the skull and a fourth sphere representing the scalp:

Zₜₒₜₐₗ=Z_{cortex}·Min(r,r_{cortex})+Z_{intermediate layers}·Max(Min(r−r_{cortex},r_{intermediate layers}−r_{cortex}),0)+Zₛₖᵤₗₗ·Max(Min(r-r_{intermediate layers},rₛₖᵤₗₗr_{intermediate layers}),0)+Zₛₖᵢₙ·Max(Min(r−rₛₖᵤₗₗ,rₛₖᵢₙrₛₖᵤₗₗ),0)

where: Zₜₒₜₐₗ is the (total) impedance as a function of r.
r is the scalar distance from the centre of the head;
Z_{cortex} is the impedance of the cortex per unit distance;
r_{cortex} is the radius of the cortex from the centre of the head;
Z_{intermediate}_{_}_{layers} is the impedance of the intermediate layers of the head between the skin and the skull per unit distance;
r_{intermediate}_{_}_{layers} is the radius of the intermediate layers from the centre of the head;
Zₛₖᵤₗₗ is the impedance of the skull per unit distance;
rₛₖᵤₗₗ is the radius of the skull from the centre of the head;
Zₛₖᵢₙ is the impedance of the skin per unit distance; and
rₛₖᵢₙ is the radius of the skin from the centre of the head.

It may be that the radius of the cortex r_{cortex} is assumed to be 0.03 m, the radius of the intermediate layers r_{intermediate}_{_}_{layers} is assumed to be 0.05 m, the radius of the skull rₛₖᵤₗₗ is assumed to be 0.065 m and the radius of the scalp r_{scalp} is assumed to be 0.084 m. It may be that the conductivity values (from which impedance values can be derived) are assumed to be 0.44 (S/m, scalp), 0.018 (S/m, skull), 1.79 (S/m, intermediate layers), and 0.250 (S/m, brain). Preferably these values are scaled to the actual size of the subject's head to provide a better approximation. It will be understood that, by assuming a spherical model of the head, a more computationally efficient analytical solution can be found.

As an alternative to such a simplified geometry, a more realistic, anatomically correct geometry may be provided. For example, an anatomically accurate model of soft head tissues can be derived from T1-weighted magnetic resonance (MR) and diffusion tensor (DT) images of the head of the subject 40 recorded by an MRI (magnetic resonance imaging) scanner. A model of the bone structure of the head of the subject 40 can be derived from a CT (computed tomography) scan. It may be that the acquisition matrices of the MRI and CT scans have a voxel size of around 1 mm³. The models of the soft head tissues and the bone structure can be used to provide an isotropic head geometry by segmenting the T1 MRI images into (for example) seven tissue types (brain grey matter, brain white matter, CSF, scalp, eyeballs, air, and skull) which are then co-registered, and combined with the CT images using segmentation and image processing (for example as described in U.S. Pat. No. 8,478,011 which is incorporated in full herein by reference). Alternatively, the geometry data can be determined by diffusion tensor imaging (DTI) of the body portion. As another alternative, a mixed adjusted average head model could be used (e.g. the MNI-152 head by Fonov et al (Fonov V, Evans A C, Botteron K, Almli C R, McKinstry R C, Collins D L. Unbiased average age-appropriate atlases for pediatric studies. Neuroimage. 2011; 54(1):313-327 which is incorporated in full herein by reference) which is an MRI volume obtained by averaging MRI images of 152 individuals). It may be that each of the determined tissue types of the more complex geometry is allocated an impedance value (e.g. some or all of the seven tissue types may be defined as mentioned above with respect to the T1 MRI images, and allocated impedance values).

In the discussion below, it will be assumed that the simplified geometry discussed above is employed, but it will be understood that any suitable more complex geometry could be (and is typically preferably) employed instead. For the more complex geometries, more computationally intensive numerical approaches (e.g. finite element analysis) may be required to derive the impedance model within the head, rather than the analytical approach made possible with the simplified geometry.

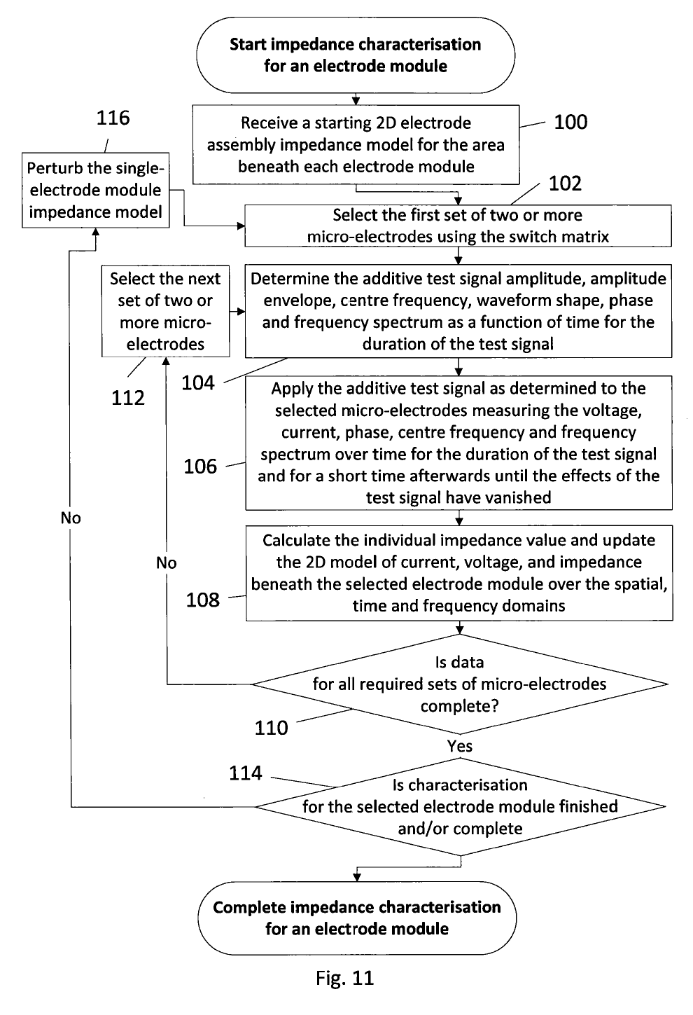

The initial impedance model considers only the impedances of the head. The impedances of the localised sub-regions within the electrolyte application region are characterised in a next step 96b. This is described below with reference to FIG. 11 with respect to electrode module 46, but the steps of FIG. 11 are repeated for electrode module 48.

In an initial step 100 of FIG. 11, the impedance is initially assumed to be uniform across the electrolyte application region 66. This uniform impedance may be represented as a (e.g. two dimensional) mathematical surface (or in some cases a three dimensional mathematical volume) which in turn is a (e.g. two or three dimensional) representation of the impedance across the electrolyte application region. The mathematical surface is initially flat (or the mathematical volume is initially uniform).

In a next step 102, a first set of two (or more) electrodes of the electrode module 46 are selected using the switch matrix 82, the set of two electrodes of the electrode module 46 comprising a test electrode and a pairing electrode. The control module 63 determines, in step 104, properties of a test signal (e.g. test signal amplitude (which may be low, say 1 mA peak), amplitude envelope, centre frequency (which may be low, say between 10 Hz and 50 Hz), waveform shape, phase and frequency spectrum as a function of time) to apply between the selected electrodes of the electrode module 46. The properties of the test signal are dependent on whether the signal generator 84 is a constant current source or a constant voltage source. If the signal generator 84 is a constant current source, then the test signal is applied by adjusting the driving voltage of the constant current source. If the signal generator is a constant voltage source, then the test signal is applied by adjusting the current output by the constant voltage source.

The determined test signal is then applied between the selected electrodes of the electrode module 46 in a next step 106, typically by superimposing the test signals on (that is, by adjusting) the stimulation signals being applied to the target treatment region of the subject 40 by way of the electrodes 70, 71 as set by step 94 (although it will be understood that test signals may alternatively be applied between the selected electrodes when no stimulation signal is being applied, for example before or after a stimulation session, or a stimulation session may be paused temporarily for the test signals to be applied). The voltage across and/or current flowing between the selected electrodes are measured by the impedance calculator 86 (which typically comprises a memory) via conductors 80 and the switch matrix 82 for the duration of the test signal and for a short time afterwards until the effects of the test signal have vanished. If the signal generator 84 is a constant current source, then at least the voltage between the selected electrodes is measured. The current may be assumed to be the driving current output by the constant current source. Alternatively, the current is also measured. If the signal generator 84 is a constant voltage source, then at least the current flowing between the selected electrodes is measured. The voltage may be assumed to be the driving voltage between the selected electrodes provided by the constant voltage source. Alternatively, the voltage across the selected electrodes is also measured.

In a next step 108, the impedance calculator 86 calculates the impedance of the electrical path between the selected electrodes from the said voltages and currents (i.e. by dividing the voltage across the selected electrodes by the current flowing between them), and stores the result in a memory (e.g. a memory of the impedance calculator 86 or of the modelling module 88), typically with reference to an impedance framework (e.g. an equivalent electrical circuit). The impedance calculator 86 will generally store the voltages measured across and currents measured flowing between the electrodes (where applicable).

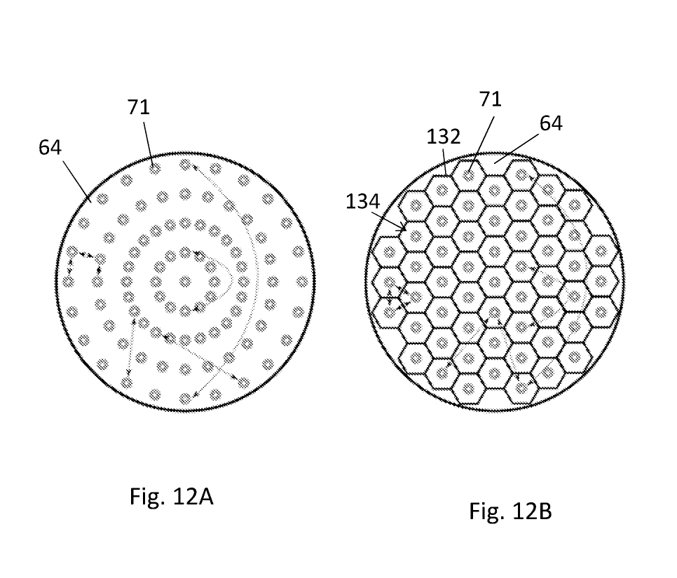

As illustrated in FIG. 12B, the electrolyte application region 66 is divided into a plurality of localised sub-regions 134, each localised sub-region 134 comprising an axial member on which two electrodes 70, 71 are mounted. The localised sub-regions 134 are physically (and electrically, within the electrolyte application region) segregated by (e.g. rubber) electrically insulating walls 132 (which in the example of FIG. 12B are hexagonal when viewed in plan from beneath) extending from the surface 64 of the first end 54 of the electrode housing and surrounding the respective segments. The walls 132 (not shown in previous figured for clarity) are configured to form a seal with the skin interface 49 when the electrode module is installed on the head. By segregating the localised sub-regions in this way, the electrolyte pathway between selected electrodes is predictable. That is, because it can be assumed that current cannot flow through the walls 132, it can be assumed that the current flows through the upper layers of the head (mostly through the skin) between the electrodes. This helps the controller to determine the current density between the electrodes of the electrode module and the skin interface. By providing walls which divide the electrolyte application region into a plurality of localised sub-regions, the electrolyte and current leakage between localised sub-regions 134 is also significantly reduced. In other embodiments (see FIG. 12A for example), the walls 132 may be omitted.

The electrodes of the first electrode module 46 can additionally or alternatively be paired with electrodes of the second electrode module 48 in order to characterise the impedance between the first end of the first electrode module 46 and the skin interface 49 using the algorithm of FIG. 11. Accordingly, it may be that each test electrode is also paired with each of the individual electrodes of the other electrode module in turn. In this case, the dominant electrical path between the electrode and the pairing electrode typically extends through a portion of the electrolyte application region of the first electrode module 46, the head and a portion of the electrolyte application region of the second electrode module 48. The impedance of each localised sub-region can be determined (e.g. by the impedance calculator 86 in step 108) in this case by pairing each said electrode with each of a plurality of different electrodes of the other electrode module, measuring the impedance of the electrical path extending between them (e.g. by measuring the voltage across the electrodes and/or current flowing between them) and comparing the measured impedances. For the purposes of characterising the impedances of the electrolyte application region, it may be that the impedance through the head between the electrodes of the first and second electrode modules 46, 48 is considered to be constant for each electrode pair between the first and second modules 46, 48. Accordingly, differences in the measured impedances are typically considered to be indicative of differences of the impedances of the localised sub-regions of the electrolyte application region comprising the respective electrodes.

It will be understood that the impedances between electrodes of the electrode module 46 (and/or the electrode module 48) and between the electrode modules 46, 48 and the skin interface may be characterised using any suitable alternative algorithm. For example, electrical stimulation signals may be applied across or between all of the electrodes of the first electrode module 46 together and all of the electrodes of the second electrode module 48 together (i.e. the electrodes of the first electrode module 46 being treated as a single electrode and the electrodes of the second electrode module being treated as a single electrode). Next, a pair of stimulating electrodes 70, 71 mounted on the same axial member in a particular segment of the electrode module 46 may be disconnected by the switch matrix 82 such that they no longer carry any current, and the change in the applied voltage (in the case of the signal generator 84 acting as a current source) or in the total current flowing between the first and second modules 46, 48 (in the case of the signal generator 84 acting as a voltage source) is measured. In the former case, a voltage increase of around V/N, where V is the original applied voltage between the first and second electrode modules 46, 48 and N is the number of segments, indicates that the impedance between the disconnected electrodes and the skin interface was average among the segments. A voltage increase which is significantly larger than V/N indicates that the current density at the stimulation segment comprising the said axial member and the said disconnected electrodes was too high in proportion to the rest of the segments. This is typically an indication that another of the segments is carrying too little current. A voltage increase which is significantly lower than V/N indicates that minimal (or no) current was being carried by that segment (localised sub-region), which is indicative that the current being carried by another segment (localised sub-region) is too high.

The measured voltage or current changes can be used to determine the impedance of the segment (localised sub-region) comprising the electrodes which were disconnected. For example, for a constant current source, the measured voltage change divided by the current flowing between the electrode modules is indicative of the impedance of the segment (localised sub-region) comprising the electrodes which were disconnected. For a constant voltage source, the applied voltage between electrode modules divided by the measured current change is indicative of the impedance of the segment (localised sub-region) comprising the electrodes which were disconnected. By (e.g. the impedance calculator 86) comparing the determined impedance with a predetermined maximum impedance of the segment (typically a maximum impedance of the electrolyte application region divided by the number of segments), it can be determined whether the current density within that segment is within an acceptable range.

It may be that the control module 63 is configured to determine a frequency response of the impedance measured between the selected electrodes. Accordingly, control module 63 may specify in step 104 that test signals of varying frequency should be applied between the selected electrodes. In one example, sawtooth signals are applied between the electrodes and the frequency response is derived from the time response of the impedance to the sawtooth signals. In addition, the phase, centre frequency and frequency content of the voltage and/or current signals are also measured by the impedance calculator 86. The frequency response of the impedance can be used to better determine which types of material are provided between the selected electrodes. For example, saline electrolyte typically has a different frequency response from human hair or air which may unintentionally be provided between selected pairs of electrodes. Information is typically provided regarding the expected frequency responses of one or more (typically two or more) types of human tissue (e.g. human hair, skin, skull, different portions of the head and/or brain), of air, and of the electrolyte in the impedance data provided by the clinician (alternatively this information may be pre-set, e.g. during manufacture). This is typically taken into account by the modelling module 88 to determine which types of material are provided between the selected electrodes.

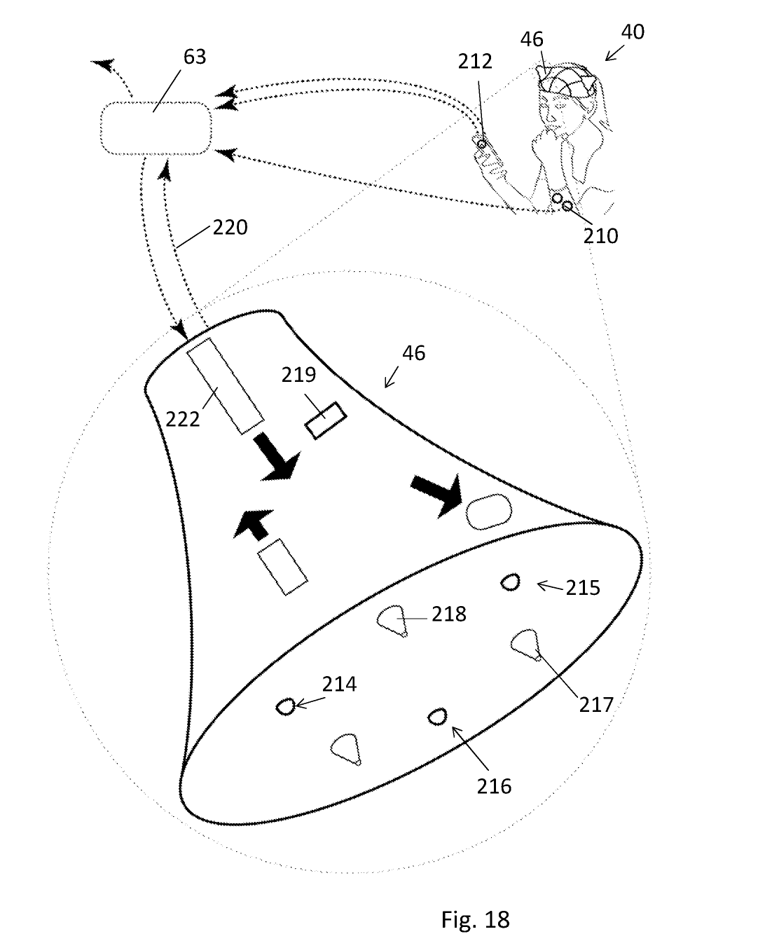

A force actuator 222 (such as a solenoid, worm drive, stepper drive, shape memory actuator, piezo actuator, MEMS thermal or magnetic actuator) may be provided (see FIG. 18), typically in the housing 52 of the electrode module 56, in order to ensure good contact between the sensors provided on the end 54 of the electrode module 46 and the skin interface 49. This can be important to prevent poor contact between the sensors and the skin interface, which may for example be caused by hair of the subject biasing the electrode module 46 away from the skin interface 49. Indeed, it may be that the control module 63 is configured to actuate (or increase the force exerted by) the force actuator 222 to push the electrode module towards the skin interface responsive to a detection from the frequency response of the determined impedances that there is hair (for example) between the electrode module and the skin interface.

Some exemplary (non-exhaustive) electrode pairings within electrode module 46 are illustrated in FIGS. 7B, 12A and 12B, FIG. 7B showing impedance vectors extending both in the plane of the surface 64 of the first end 54 of the electrode module 46 and in an axial direction parallel to the line of shortest distance between the first and second ends 54, 56 of the housing 52 of the electrode module 46, FIGS. 12A and 12B (being plan views) showing impedance vectors extending only in the plane of the surface 64 of the first end 54 of the electrode module 46. As shown by the dotted lines terminated in arrow-heads of FIG. 7B, which illustrate example impedance paths 120-126 between various pairs of electrodes 70, 71 of the electrode module 46, it is possible to perform impedance measurements in three dimensions (because the electrodes of the electrode module 46 are horizontally and vertically distributed). By measuring impedances between electrodes which are spaced from each other in an axial direction, impedances between those electrodes (and between those electrodes and the skin interface 49) can be determined. High impedance values may be indicative of dry patches, which cause electrolyte adjacent to the dry patches to carry more current than intended. Accordingly, these measurements are typically indicative of whether the current density at the skin interface in each localised sub-region is at an acceptable level, or whether it is at a level which should be reduced (e.g. by adding more electrolyte or reducing stimulation). Although a detailed three dimensional model of impedance (and voltage and current) variations as a function of position throughout the electrolyte application region 66 can be determined in this way, in the present example a simpler two dimensional model (or mathematical surface) of impedance (and voltage and current) variations across the electrolyte application region 66 as a function of position is provided (in a plane parallel to the plane of the surface 64 of the first end of the electrode module 46 which defines the electrolyte application region 66).

In a next step 110, it is determined whether the impedance data has been obtained for all required sets of micro-electrodes. Typically, for a full characterisation, each electrode is selected as the test electrode in turn and, in each case, the test electrode is paired with each of the other electrodes of the electrode module 46 in turn (and optionally with each of the electrodes of the electrode module 46). If not every required pair has been tested, the next pair of electrodes is selected in step 112 and then steps 104 to 110 are repeated for that pair. If impedance data from all required pairs of electrodes has been obtained, the impedances calculated from the voltage and/or current measurements (and/or the voltage and/or current measurements themselves) are provided to the modelling module 88 and a check is then made in step 114 as to whether the impedance model of the electrolyte application region (starting from the initial impedance model which assumes that the impedance is uniform across the electrolyte application region) conforms to the impedances calculated from the current and/or voltage measurements. This may be done by checking whether an (e.g. least squares) objective function between the impedances predicted by the model and the impedances calculated from the measurements meet one or more accuracy criteria (e.g. whether the error satisfies a completion value of the objective function). If the impedance model does not conform to the impedance measurements, the modelling module 88 perturbs the impedance model by adjusting the flat impedance surface of electrolyte application region in a next step 116 to better match the impedance measurements. The impedance surface may be adjusted to increase the impedance of one or more localised sub-regions of the surface, and/or to decrease the impedance of one or more localised sub-regions. Any suitable mathematical optimisation technique may be used (e.g. downhill simplex, conjugate gradient, finite mesh) and the nature of the perturbations is specific to the mathematical optimisation method used. Then, steps 102-114 are repeated iteratively until the impedance model of the electrolyte application region matches the measurements to a sufficient degree of accuracy (i.e. until the objective function meets one or more accuracy criteria—e.g. until the value of the objective function is less than a predetermined threshold).

Thus, by measuring impedances between multiple pairs of electrodes (both within each electrode module and between electrode modules), the modelling process can determine the impedance within a plurality of localised sub-regions of each of the electrolyte application regions between the electrode modules 46, 48 to a high level of accuracy. This also provides higher resolution impedance data than can be achieved with existing electrode modules which comprise only a single electrode. This allows dry patches to be determined at localised sub-regions of the electrolyte application regions which would not otherwise be detectable, and corrective action can then be taken (see below).

A model of current density in the electrolyte application region 66 (and optionally within the head) may be generated together with the impedance model (e.g. derived from the same measurements used to determine the impedance model) or derived from the impedance model.

Referring back now to FIG. 10, when the impedances between each of the electrode modules 46, 48 and the skin interface 49 have been characterised using the algorithm of FIG. 11, in a next step 96c the control module 63 determines properties of a test signal (e.g. test signal amplitude, amplitude envelope, centre frequency, waveform shape, phase and frequency spectrum as a function of time) to apply between the electrode assemblies 46, 48 as a whole (such that the electrodes of the first electrode module 46 can be treated as a single electrode, and the electrodes of the second electrode module 48 can be treated as a single electrode). The determined test signals are then applied between the electrode modules 46, 48 in a next step 96d, typically by superimposing the test signals on (that is, by adjusting) the stimulation signals being applied to the target treatment region of the subject 40 by way of the electrodes of the electrode modules 46, 48 (although it will be understood that test signals may alternatively be applied between electrode modules when no stimulation signal is being applied). The voltage across and/or current flowing between the electrodes of the electrode modules 46, 48 are measured (in bulk—i.e. the voltage across the electrode modules 46, 48 as a whole and the current flowing between the electrode modules 46, 48 as a whole) by the impedance calculator 86 for the duration of the test signal and for a short time afterwards until the effects of the test signal have vanished. Typically, the phase, centre frequency and frequency content of the voltage and/or current signals are also measured by the impedance calculator 86.

As above, the test signal is typically applied by the signal generator 84 in a current source mode, but alternatively it may be that the test signals are applied by the signal generator 84 in a voltage source mode.

The majority of the test signals applied between the electrode modules 46, 48 typically flow from one module to the other through the head (and typically the target treatment region inside the brain), but it may be that a portion of the electrical current is shunted across the skin interface 49 between the electrode modules 46, 48 (without passing through the head).

In a next step 96e, the impedance calculator 86 of the first electrode module 46 (or alternatively the impedance calculator 86 of the second electrode module 48, or an impedance calculator 86 provided externally to the first and second electrode modules 46, 48) calculates the impedance of the electrical path between the electrode modules 46, 48, and stores the result in a memory in step 96f (e.g. a memory of the impedance calculator 86 or of the modelling module 88). If a measure of the shunt impedance across the skin interface is available (see below), that will be taken into account in step 96e.

In a next step 96g, the impedance calculated from the measured voltages and/or currents is provided to the modelling module 88 of the first electrode module 46 (or alternatively the modelling module of the second electrode module 48 or of a modelling module external to the first and second electrode modules 46, 48) and a check is made as to whether the impedance model (i.e. comprising both the impedance model of the head and the impedance models of the electrolyte application regions between the electrode modules 46, 48 and the skin interface 49) conforms with the impedance calculated from the measured currents and/or voltages. This may be done by checking whether an (e.g. least squares) objective function between the impedance predicted by the model and the impedance calculated from the voltage and/or current measurements meet one or more accuracy criteria. If the impedance model does not conform to the measurements, the impedance model is perturbed by the modelling module 88 in a next step 96h to better match the voltage and current measurements. In this case, the perturbation of the model may comprise adjustments to any one or more of the parameters of the analytical expression provided above for the impedance through the head (Zₜₒₜₐₗ). Any suitable mathematical optimisation technique may be used (e.g. downhill simplex, conjugate gradient, finite mesh) and the nature of the perturbations is specific to the mathematical optimisation method used. Then, steps 96c to 96h are repeated iteratively until the model meets the accuracy criteria.

As indicated above, if one or a plurality of the impedances within an electrolyte application region is outside of a predetermined safe range, it may be indicative that the current density at another part of the electrolyte application region exceeds a safe range. Accordingly, referring back to FIG. 9, in a next step 140, the control module 63 determines whether any of the impedances calculated from the said voltage and/or current measurements are outside of a predetermined safe range (e.g. below a predetermined safe threshold). If one or more of the impedances are outside of the predetermined safe range, it may be that the control module 63 aborts the stimulation being applied to the subject 40 as a safety precaution in step 142. If none (or fewer than a threshold number) of the impedances in the electrolyte application region calculated from the said voltage and/or current measurements are outside of the predetermined safe range, it is then checked in step 144 whether any of the impedances of the electrolyte application regions are outside a predetermined working range (which is different from the predetermined safe range, e.g. a lower predetermined threshold). Typically impedances between the upper limit of the working range and the upper limit of the safety range do not present a safety risk, but are undesirable. Accordingly, if any of the impedances of the electrolyte application regions are outside of the predetermined working range, this is reported to the clinician in step 146 (e.g. by way of an audible, tactile or visual alarm) and the algorithm proceeds to step 147 (see below). If none of the impedances of the electrolyte application regions are outside of the predetermined working range, the algorithm proceeds straight to step 147.

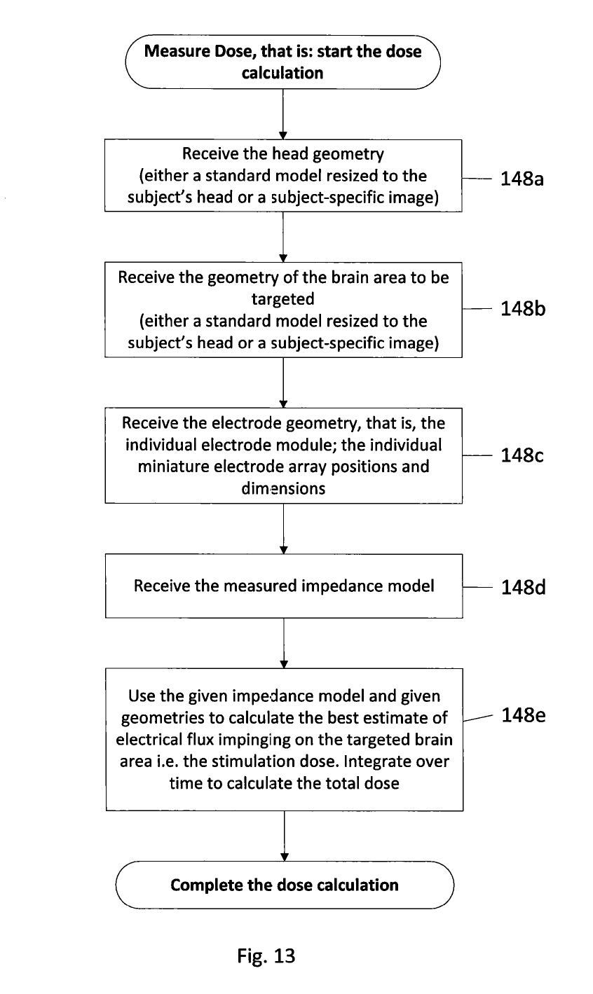

In parallel with steps 140-146, the control module 63 is configured to calculate, in step 148, a dosage of electrical stimulation applied to the target treatment region inside the brain by way of the stimulation signals applied to the skin interface by the electrodes of the electrode modules 46, 48. Step 148 is described in more detail by the flow diagram of FIG. 13.

In step 148a, the control module 63 receives the head geometry (which as discussed above may be a standard simplified geometry of the head resized with the actual size of the head of the subject 40, determined by measurement or from an image of the subject's head, or a subject-specific image of the head) and in step 148b the control module 63 receives the predetermined data indicative of the position of the target treatment region within the head (which, again, may be standard predetermined data which is not specific to the subject resized with the actual size of the head of the subject 40, determined by measurement or from an image of the subject's head, or a subject-specific image of the head including the target treatment region), both of which were typically input by the clinician.

As discussed above, it may be that the geometry data assumes a simplified geometry of the head, for example the geometry data may assume that the head comprises four concentric spheres, each sphere representing a different portion of the head (one for the cortex, one for the intermediate layers between cortex and skull, one for the skull and one for the scalp). Alternatively the geometry data may represent a more complex, anatomically correct geometry. Whether or not the geometry data describes a simplified or more accurate geometry of the head, an anatomically accurate image (e.g. MRI scan) of the head of the subject 40 may be used together with the predetermined data to more accurately identify the location of the target treatment region within the head of the subject 40. For example, in order to identify the location of the target treatment region of the head, an MRI image of the head of the subject 40 may be processed with a semi-automated image segmenting tool based on expert neuroanatomist rules (e.g. Fallon-Kindermann rules), such as the semi-automatic segmenter described in Al-Hakim, Ramsey, et al. “A dorsolateral prefrontal cortex semi-automatic segmenter.” Medical Imaging. International Society for Optics and Photonics, 2006 which is incorporated herein in full by reference.

In a next step 148c, the control module 63 receives the electrode geometry, i.e. the positions of the electrode modules 46, 48 on the head and the positions and dimensions of the electrodes 70, 71 within the electrode arrays of the first and second modules 46, 48 (typically relative to the received head geometry). This may be indicated by the clinician or by the subject; alternatively the electrode modules 46, 48 may be positioned at a predetermined location on the head which is pre-set in the control module 63. In a next step 148d, the control module 63 receives the impedance model (comprising the impedance models of the electrolyte application regions and the impedance model of the head) determined in step 96. Then, in step 148e, the control module 63 scales the received impedance model in accordance with the received head geometry (which is typically sized in accordance with the patient's head) to better tailor the impedance model to the subject 40. For example, the values of the radii of the cortex, the intermediate layers, the skull and the skin may be scaled in accordance with the corresponding measured radii of the subject. The control module 63 then models the electrical field applied through the head portion comprising the target treatment region as a result of the electrical stimulation (in accordance with the schedule defined by the clinician) being applied at the received positions of the electrode modules 46, 48 and electrodes 70, 71 using the scaled impedance model. From the electric field model, the control module 63 determines the instantaneous electrical stimulation dosage impinging on the target treatment region by performing a volume integration of the electric field through the target treatment region.

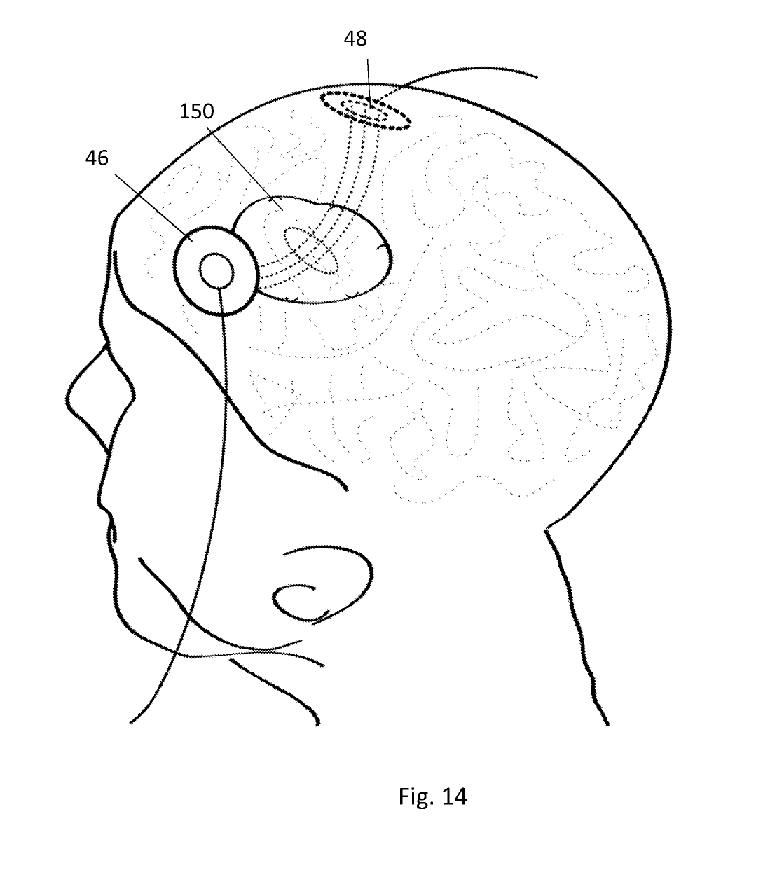

Step 148e is illustrated in FIG. 14, which is a visual representation of the electric field model, and shows the target treatment region 150 (which in this case is the left dorsolateral prefrontal cortex (DLPFC), which can be targeted for the treatment of depression) and the current flowing between the electrode modules 46, 48 (shown schematically in FIG. 14) which impinges on the target treatment region 150.

The instantaneous dosage calculated in step 148e may be integrated over time to calculate a total dosage applied to the target treatment region.

As discussed, it may be that a portion of the current applied to the head does not penetrate the skin interface 49 and is instead shunted across the skin interface 49 between the first and second electrode modules 46, 48. It may be that this shunt current is measured (see below) and taken into account in the calculation of the dosage of electrical stimulation impinging on the target treatment region. For example, the calculated dosage may be scaled in accordance with the proportion of the current applied to the skin interface 49 by the electrodes of the electrode modules 46, 48 which is shunted across the skin interface 49.

As discussed, different types of human tissue (e.g. skin, hair, bone, different portions of the head and/or brain) have different frequency responses and, typically, the impedance data provided by the clinician comprises data regarding the expected frequency responses of different types of human tissue. It may be that step 96 of FIG. 10 further comprises applying test signals between the electrode modules 46, 48 of different frequencies between different selected pairs of electrodes and it may be that the modelling module 88 is configured to calculate the impedances of different types of tissue within the head of the subject from the response of the measured impedances to test signals of different frequencies, taking into account the impedance data.

As an alternative to calculating the dosage of electrical stimulation applied to the head of the subject using the method described with reference to FIG. 13, the following method may be performed (e.g. by the control module 63). Indeed, the following method may be used to estimate the dosage of electrical stimulation applied to a target treatment region between electrode modules which each comprise only a single electrode (because it does not rely on the detailed impedance characterisations of the electrolyte application regions discussed above). The method comprises mathematically modelling an electric field applied through the head as a function of position responsive to an electrical stimulation applied to the skin interface 49 by the electrodes of electrode modules 46, 48, using the said geometry data and the impedance data (and typically the predetermined data indicative of the position of the target treatment region within the head).

As discussed above, it may be that the geometry data assumes a simplified geometry of the head (e.g. the “4-shell model” described above), or a more realistic, anatomically correct geometry may be provided.

The impedance data may assume that the impedance of the tissue of the head is homogeneous between the electrode modules 46, 48, but preferably the impedance data comprises impedance information relating to a plurality of different types of tissue in the electrical path through the head between the electrode modules. Typically the impedance data is related to the geometry data (e.g. different impedance values are associated with different regions of the head defined by the geometry data).

The location of the target treatment region is determined within the head geometry from the said predetermined data indicative of the position of the target treatment region within the head.

As the stimulation frequencies are typically low (typically <100 Hz), the stimulation physics are described adequately by the quasi-static conduction (QSC) approximation to Maxwell's equations:

Δ×E=0

Δ×H=J

where E is the electric field, H is the magnetic field, J is the current density and Δ x is the Maxwell curl operator, of which those skilled in the art would be aware (see e.g. “Quasi-static approximations of Maxwell equations”, G. Rubinacci, F. Villone, March 2002)

To relate the stimulation current applied to the scalp between the electrode modules 46, 48 to the electric field applied through the head as a result of the said applied stimulation currents, the “forward problem” of the above equations needs to be solved to determine the electric field (voltage) and current distributions within the head. There are a number of standard solutions to the “forward problem” which would be known to one skilled in the art, including: perturbation analysis of quasi-analytical solutions (see G. Nolte and G. Curio, Perturbative solutions of the electric forward problem for realistic volume conductors, J. Appl. Phys. 86(5), 1999, pp. 2800-2812, which is incorporated herein in full by reference); boundary element methods (BEM) (see J. Kybic, M. Clerc, T. Abboud, O. Faugeras, R. Keriven and T. Papadopoulo, A common formalism for the integral formulations of the forward EEG problem, IEEE Trans. Med. Imag., 24(1), 2005, pp. 12-18, which is incorporated herein in full by reference); and 3D discretization methods like Finite Difference (FD), Finite Volume (FV) and Finite Element (FE) methods (see, for example, S. Lew, C. H. Wolters, T. Dierkes, C. Mier, and R. S. MacLeod, Accuracy and run-time comparison for different potential approaches and iterative solvers in finite element method based EEG source analysis, Applied Numerical Mathematics, 59(8), 2009, pp. 1970-1988, which is incorporated herein in full by reference). As these approaches would be known to one skilled in the art, they are not set out again here for the sake of brevity.

Each of these techniques takes the geometry data and the impedance data as inputs. Boundary conditions are also typically provided, including any one or more of: the measured voltages and currents at each of the electrodes; any known voltages and currents determined during measurement of the current shunted across the surface of the scalp (see below); an assumption that no current flows from the skin into the surrounding air; and an assumption that no current disperses from the head and into the neck. Also typically provided as inputs are the locations of the electrode modules 46, 48; alternatively, the positions of the electrode modules 46, 48 may be pre-defined by the solution to the forward problem.

Typically for simpler head geometries, analytical or quasi-analytical approaches can be used to solve the forward problem, and for more complex geometry data, more computationally intensive numerical approaches (such as finite element analysis) are required.

Other relevant literature in this field includes: Turovets S, Volkov V, Zherdetsky A, Prakonina A, Malony A D. A 3D Finite-Difference BiCG Iterative Solver with the Fourier-Jacobi Preconditioner for the Anisotropic EIT/EEG Forward Problem. Computational and Mathematical Methods in Medicine. 2014; 2014:426902. doi:10.1155/2014/426902; Soleimani, Manuchehr, et al. “Electrical impedance tomography guided cryosurgery for the brain using a temporally correlated image reconstruction.” XXIX General Assembly of the International Union of Radio/Union Radio Scientifique Internationale (2008); Baillet, Sylvain, John C. Mosher, and Richard M. Leahy. “Electromagnetic brain mapping.” Signal Processing Magazine, IEEE 18.6 (2001): 14-30; da Silva Caeiros, Jorge Manuel. “Electromagnetic Tomography: Real-Time Imaging using Linear Approaches.” (2010); Turovets S, Volkov V, Zherdetsky A, Prakonina A, Malony A D. A 3D Finite-Difference BiCG Iterative Solver with the Fourier-Jacobi Preconditioner for the Anisotropic EIT/EEG Forward Problem. Computational and Mathematical Methods in Medicine. 2014; 2014:426902. doi:10.1155/2014/426902; and Windhoff, Mirko, Alexander Opitz, and Axel Thielscher. “Electric field calculations in brain stimulation based on finite elements: an optimized processing pipeline for the generation and usage of accurate individual head models.” Human Brain Mapping 34.4 (2013): 923-935, all of which are incorporated herein in full by reference.

In order to estimate the dosage of electrical stimulation being applied to the target treatment region, a volume integration of the determined electric field through the target treatment region is performed, for example:

Dᵢ(t)=∫∫∫I(x,y,z)·1_{Target}(x,y,z)dx dy dz

where the volume integration is over the head volume; I(x, y, z) is the current density as a function of position and 1_{T}(x, y, z) is an indicator function indicating that we are in the chosen target treatment area (e.g. the dIPFC).

Additionally, by integrating this value over the treatment session time, the total dosage applied to the target treatment region can be determined, for example:

D=∫[∫∫∫I(x,y,z,t)·1_{Target}(x,y,z)dx dy dz]dt

where I in this case is also a function of time.

The measured skin shunt current shunted across the skin interface between the electrode modules 46, 48 may be taken into account when determining the electric field through the head (e.g. the electric field intensity may be scaled according to the proportion of the applied stimulation lost as the skin shunt current); alternatively, the measured skin shunt current shunted across the skin interface between the electrode modules 46, 48 may be taken into account when determining the dosage of electrical stimulation applied to the target treatment region. For example, the dosage of electrical stimulation applied to the target treatment region may be determined from the electric field data, and the dosage value may be scaled according to the proportion of the applied stimulation lost as the skin shunt current.

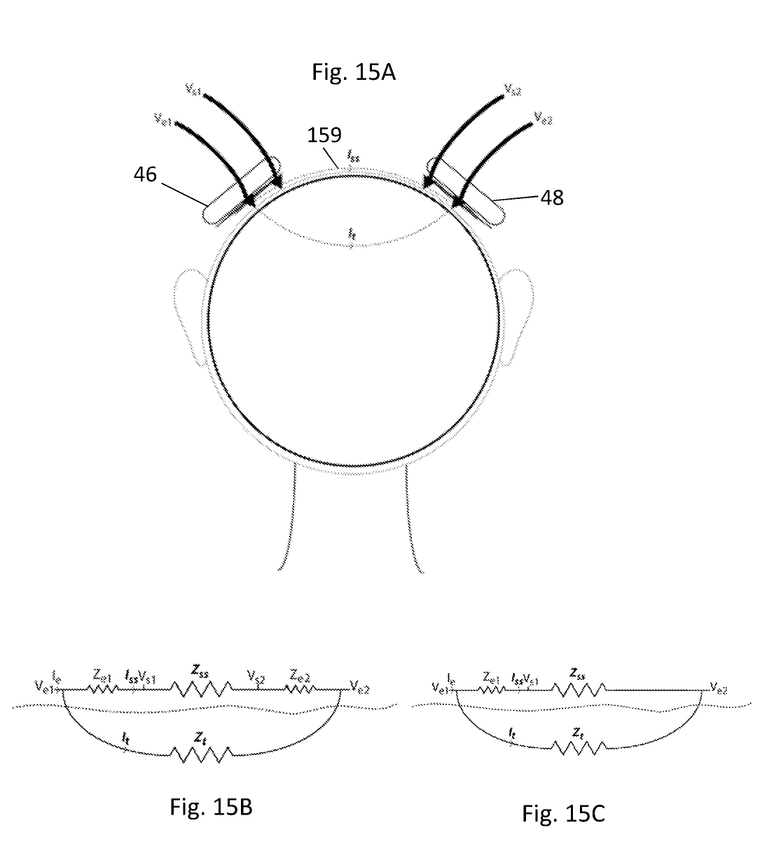

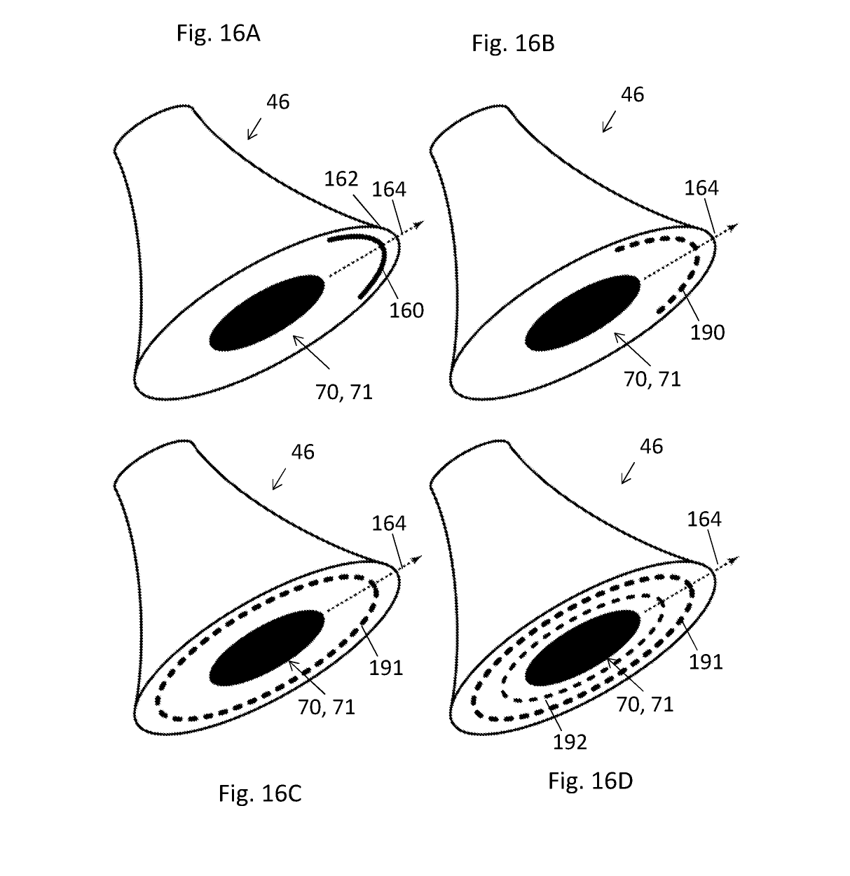

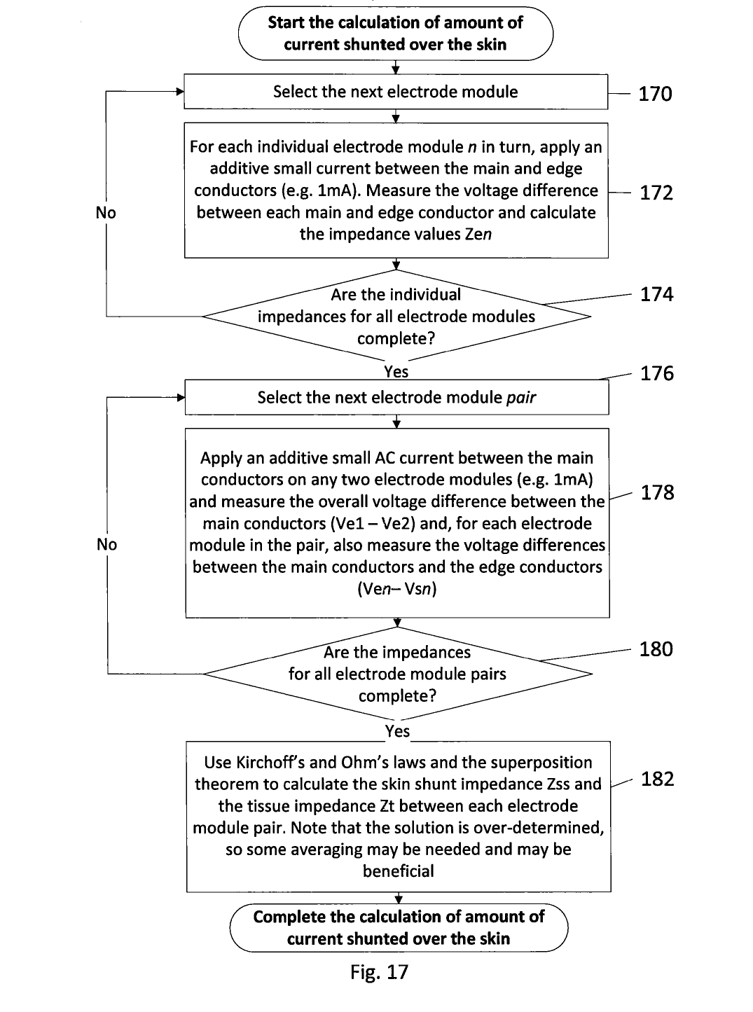

The current 159 shunted across the skin interface 49 between the first and second electrode modules 46, 48 (see FIG. 15A) can be measured by providing one or more shunt measurement conductors on a shunt path extending along the skin interface between the modules 46, 48. As shown in FIG. 16A, which is a schematic perspective view of the electrode module 46, an arced shunt measurement conductor 160 extends partially around the electrodes 70, 71 (which are illustrated by a black circle in FIG. 16A) and is positioned adjacent to an edge 162 of the perimeter of the surface 64 of the first end 54 of the electrode module 46 which defines the electrolyte application region between the electrode module 46 and the skin interface 49. A dotted line terminated with an arrow 164 in FIG. 16A illustrates the direction of shunt current flow across the skin interface between the electrode module 46 and electrode module 48. Typically the second electrode module 48 also has a shunt measurement conductor 160, which is positioned adjacent to an edge of the first end of the second electrode module 48 closest to the first electrode module 46. An algorithm for measuring the current shunted across the skin interface 49 between the first and second electrode modules is illustrated in FIG. 17.

In a first step 170, one of the electrode modules, say the first electrode module 46, is selected. In a next step 172, an AC test current is applied by the control module 63 between the electrodes of the electrode module 46 and the shunt measurement conductor 160 of the first electrode module 46, and the voltage difference between the electrodes and the shunt measurement conductor 160 (of the first electrode module 46) responsive to the test signal is measured. The test current may be applied as a momentary incremental change to the stimulation signals applied by the electrodes during a stimulation session. The voltage difference is divided by the test current to determine the impedance between the electrodes 70, 71 and the shunt measurement conductor 160. In a next step 174, it is determined whether steps 170, 172 have been performed in respect of each of the electrode modules 46, 48 (and any other electrode modules provided). If not, steps 170, 172 are repeated for the next electrode module. If steps 170, 172 have been performed for each of the electrode modules 46, 48, the control module 63 selects a pair of electrode modules 46, 48. In a next step 178, an AC test current is applied between the electrodes of the first electrode module 46 and the second electrode module 48 and the overall voltage difference between the modules 46, 48 responsive to the test signal is measured. Again, the test current may be applied as a momentary incremental change to the stimulation signals applied during a stimulation session. The voltage differences between the electrodes of the first electrode module 46 and the shunt conductor 160 of the first electrode module, and between the electrodes of the second electrode module 48 and the shunt conductor 160 of the second electrode module 48 responsive to the test current are also measured. In step 180, it is determined whether steps 176, 178 have been performed for each pair of electrode modules. In this example, only one pair of electrode modules 46, 48 is provided, but it will be understood that if any additional electrode modules were provided, steps 176, 178 would be repeated for each possible pair of electrode modules. When steps 176, 178 have been performed for all possible pairs of electrode modules, a skin shunt impedance Zss and a tissue impedance Zt between each pair of electrode modules can be determined, for example as follows.

In order to determine the skin shunt impedance Zss and tissue impedance Zt between the electrode modules 46, 48, the equivalent electrical circuit of FIG. 15B can be considered. Ve1 and Ve2 in the equivalent circuit of FIG. 15B refer to the potentials at the electrodes of the first electrode module 46 (Ve1) and at the electrodes of the second electrode module 48 (Ve2). Vs1 is the potential at the shunt measurement conductor 160 of the first electrode module 46 and Vs2 is the potential at the shunt measurement conductor 160 of the second electrode module 48. Ze1 is the impedance between the electrodes and the shunt measurement conductor 160 of the first electrode module 46 and Ze2 is the impedance between the electrodes and the shunt measurement conductor 160 of the second electrode module 48. Ie is the current applied between the first and second electrode modules 46, 48.

Ohm's law can be applied as follows:

(Vₑ₁−Vₛ₁)/Zₑ₁=Iₛₛ

(Vₛ₂−Vₑ₂)/Zₑ₂=Iₛₛ

where Iₛₛ is the current shunted across the skin interface 49 between the first and second electrode modules 46, 48.

As all of Vₑ₁, Vₑ₂, Vₛ₁, Vₛ₂, Zₑ₁ and Zₑ₂ are known, Iss can be determined from either or both of the above equations. Typically, Iss is determined from both equations and an average (e.g. mean) value taken.

Kirchoff's current law can also be applied as follows:

Iₑ=Iₛₛ+Iₜ

where Iₜ is the current flowing through the head

As Iₑ and Iₛₛ are known, Iₜ can be determined from this equation.

Zₛₛ can be calculated as follows:

(Vₛ₁−Vₛ₂)/Iₛₛ

Zₜ can be calculated as follows:

(Vₑ₁−Vₑ₂)/Iₜ

In other embodiments, it may be that only one of the first and second electrode modules 46, 48 is provided with a shunt measurement conductor 160. For example, it may be that only the first electrode module is provided with a shunt measurement conductor 160. In this case, steps 170, 172 are performed only in respect of the first electrode module 46 and the impedance Ze1 between the electrodes and the shunt measurement of the first electrode module 46 is calculated. In addition, in step 178, the voltage difference between the electrodes of the electrode modules 46, 48 responsive to the test current signal is measured as before, but the voltage between electrodes and shunt measurement conductor is only measured in respect of the first electrode module 46. In this case, the alternative equivalent electrical circuit shown in FIG. 15C can be used in step 182 to determine the shunt current Iss, the shunt impedance Zₛₛ and the tissue impedance Zₜ. Ohm's law can be applied as follows:

(Vₑ₁−Vₛ₁)/Zₑ₁=Iₛₛ

As all of Vₑ₁, Vₛ₁, and Zₑ₁ are known, I_{SS} can be determined from the above equation.

Kirchoff's current law can also be applied as follows:

Iₑ=Iₛₛ+Iₜ

As Iₑ and Iₛₛ are known, Iₜ can be determined from this equation.

Zₛₛ can be calculated as follows:

Zₛₛ=(Vₛ₁−Vₑ₂)/Iₛₛ

Zₜ can be calculated as follows:

Zₜ=(Vₑ₁−Vₑ₂)/Iₜ

In each case, the characterisation algorithm of FIG. 17 may be run iteratively for different frequencies; waveforms; amplitudes or other dimensions to derive a multi-dimensional characterisation of the skin shunt effect. Since the algorithm may be run very quickly, with thousands of iterations per second, this multi-dimensional characterisation can easily be achieved with fine granularity in less than a second.

As shown in FIG. 16B, the shunt measurement conductor 160 of FIG. 16A may be replaced by a plurality of shunt measurement conductors 190 spaced apart from each other in an arced arrangement similar to that of the shunt measurement conductor 160 of FIG. 16A. By spacing the shunt measurement conductors apart from each other, the shunt measurement conductors can be made smaller so that they have less effect on the shunt current flowing between the first and second electrode modules 46, 48 (whereas the larger conductor 160 may cause an effective equipotential on its surface which affects the shunt current). In addition, by providing multiple shunt measurement conductors, finer resolution measurements can be made and an indication of the shunted current direction can be determined (e.g. by detecting current flowing through one or more of the conductors but not through one or more other said conductors).

FIG. 16C shows another alternative shunt measurement conductor arrangement, namely that a plurality of shunt measurement conductors 191 spaced apart from each other and arranged around the electrodes in a circle. A benefit of this arrangement is that the general direction of the shunt current does not need to be estimated in advance, and it can instead be determined by measurement. This may be particularly advantageous if more than two electrode modules are provided and shunt current flows in more than one direction across the skin interface between different pairs of electrode modules.

FIG. 16D shows the shunt measurement conductor arrangement of FIG. 16C as a first shunt measurement conductor arrangement 191 but with a second circular measurement conductor arrangement 192 provided around the electrodes but having a smaller diameter than the first shunt measurement conductor arrangement. This allows finer-grained modelling and more accurate calculation of the shunt current and the shunt impedance. Alternatively, the current flowing between the first and second shunt measurement conductor arrangements can be used to estimate the current shunted across the skin interface between the first and second electrode modules 46, 48 without having to perform the detailed calculations described above.

Referring back to FIG. 9, when the electrical stimulation dosage impinging on the target treatment region 150 has been calculated in step 148 (preferably taking into account the measured current shunted across the skin interface between the electrode modules 46, 48), in a next step 200 a check is made to determine whether the dosage of electrical stimulation impinging on the target treatment region 150 exceeds a safe range. If so, the algorithm aborts the stimulation in step 142. If not, a further check is made in step 202 to determine whether the calculated electrical stimulation dosage falls outside a working range. These checks may be made for either or both of the instantaneous and cumulative dosage. An electrical stimulation dosage between the upper limit of the working range and the upper limit of the safe range does not present a safety risk, but is undesirable. Accordingly, the algorithm provides a (e.g. tactile, visual and/or audible) warning to the clinician and/or the subject in step 204 and proceeds to step 147 (see below). If the dosage does not fall outside the working range, the algorithm proceeds straight to step 147.

As indicated above, in parallel with step 96, the control module 63 is configured to measure in step 98 one or more physiological stress indicators to determine whether the subject 40 is experiencing or is likely to experience a form of physiological stress (e.g. a fit) responsive to the stimulation being applied to the head by the electrode modules 46, 48. In particular, early stress indicators of which the subject 40 is not yet aware can be detected by appropriate sensors, which can enable the prevention of side effects. With reference to FIG. 18, a plurality of sensors is provided, each of which is configured to measure a physiological stress indicator of the subject 40. The plurality of sensors comprises an optical heart rate monitor 210 mounted to the subject's wrist by way of a wrist band; an accelerometer and/or gyroscope 212 provided in the subject's mobile device 44; three colourimeters 214, 215, 216 comprising an LED and photodetector provided on (and being spaced apart from each other at) the said end 54 of the housing 52 of the electrode module 46; a temperature sensor 217 provided on the said end 54 of the housing 52 of the electrode module 46; and a pH sensor 218, also provided on the said end 54 of the housing 52 of the electrode module 46 and being spaced apart from the temperature sensor 217 and the colourimeters 214-216. Additionally or alternatively, an accelerometer and/or gyroscope 219 may also be provided in the electrode module 46. A pulse oximeter may also be mounted to the user's wrist or fingertip. The sensors may be in wireless data communication with the control module 63 via a wireless (e.g. Bluetooth, wi-fi) link 220; alternatively the connection between the sensors and the control module is wired (e.g. using conductors 60, 62 or alternative conductors).

The optical heart rate monitor 210 is configured to determine the (or changes in the) heart rate of the subject 40, which (e.g. if the heart rate or changes in heart rate are unusually high) may be an indicator of physiological discomfort or of a pre-ictal state of the subject 40 as a side effect of electrical stimulation being applied to the subject 40 by the electrode modules 46, 48. It may be that the measured heart rate is used to calculate a heart rate variability statistic, and it may be that the heart rate variability statistic (e.g. beyond a limit) provides the said indicator of physiological discomfort or pre-ictal state (e.g. a sudden change in heart rate may be a possible pre-cursor to a fit—this may be determined from the heart rate variability (HRV) statistic).

The accelerometer and/or gyroscope 212 provided in the subject's mobile device 44 and/or the accelerometer and/or gyroscope provided in the electrode module 46 are configured to detect movements of the subject 40 indicative of a physiological stress of the subject, in particular movements which indicate a pre-ictal state of the human subject (e.g. pre-epileptic fit) or movements which indicate that the human subject is slumping or having a fit or a seizure or small tremor movements (which can occur as a side-effect of electrical stimulation being applied to the subject 40 by the electrode modules 46, 48) which provide a physiological warning indicator.

The colourimeters 214-216 are configured to measure one or more physiological stress indicators indicative of a skin sensitivity of the human subject, which may be affected by, for example, the current density impinging on the skin interface. The colourimeters are configured to measure a parameter indicative of a colour of the body portion (e.g. of the skin interface) by directing light from the LED which has a wavelength in the region 620 nm to 750 nm (red light), or in the infrared region, towards, and using the photodetector of the colourimeter to receive reflected light from, the skin interface, the quantity of light received by the photodetector being indicative of a redness of the skin. The redness of the skin is typically an indicator of skin sensitivity. It may also be that skin redness is a pre-cursor to skin lesions forming. Accordingly, a measure of skin redness exceeding a threshold value may be an indicator of a skin sensitivity of the subject 40 (which can occur as a side-effect of electrical stimulation being applied to the subject 40 by the electrode modules 46, 48).

The temperature sensor 217 is configured to measure a temperature of the skin interface 49. A high skin temperature (e.g. a skin temperature exceeding a threshold value) or quick changes in the skin temperature may be an indication of skin sensitivity of the human subject which occurs as a side-effect of electrical stimulation being applied to the subject 40 by the electrode modules 46, 48.

The pH sensor 218 is configured to measure a pH of the skin interface. A skin pH which lies outside of an acceptable range may be an indicator of a physiological stress of the human subject which occurs as a side-effect of electrical stimulation being applied to the subject 40 by the electrode modules 46, 48.

The pulse oximeter (where provided) is configured to measure the blood oxygen saturation at the skin interface, a blood oxygen saturation which lies outside of an acceptable range being an indicator of skin sensitivity of the human subject which occurs as a side-effect of electrical stimulation being applied to the subject 40 by the electrode modules 46, 48.

It may be that the electrodes of the electrode module 46 can be configured (e.g. by the control module 63) to operate in an electroencephalography (EEG) mode. In this case, it may be that the electrodes of the electrode module can operate as an EEG sensor for measuring one or more physiological stress indicators of the subject (e.g. by detecting a pre-migraine aura from EEG markers).

By detecting one or more physiological stress indicators indicative of a side effect of the human subject due to transcranial stimulation, or at an early stage of discomfort experienced by the subject, corrective action can be taken early (see below).

By providing a plurality of sensors spaced out over the said first end 54 of the electrode module 46, one or more physiological stress indicators can be measured at each of a plurality of different localised sub-regions of the electrolyte application region 66. This information can be used to take corrective action specific to the localised sub-region where a physiological stress indicator meeting one or more physiological stress criteria (e.g. the skin redness measured by one of the skin colourimeters exceeds a threshold value) was measured.

By providing a plurality of different types of sensors, different types of physiological stress can be detected. It may be that one type of physiological stress occurs before others (or in the absence of others). Accordingly, detecting different types of physiological stress allows physiological stress to be detected earlier than might otherwise be the case and/or allows a fuller picture of stress and total side effect to be obtained and acted upon.

The control module 63 is also configured to receive input entered by the subject on the mobile device 44 indicative of any physiological stress they are experiencing. For example, the mobile device 44 may be running an application which allows the subject to enter input using a visual analogue scale (VAS) or similar for several variables including pain, irritation, discomfort, stress and pre-migraine aura.

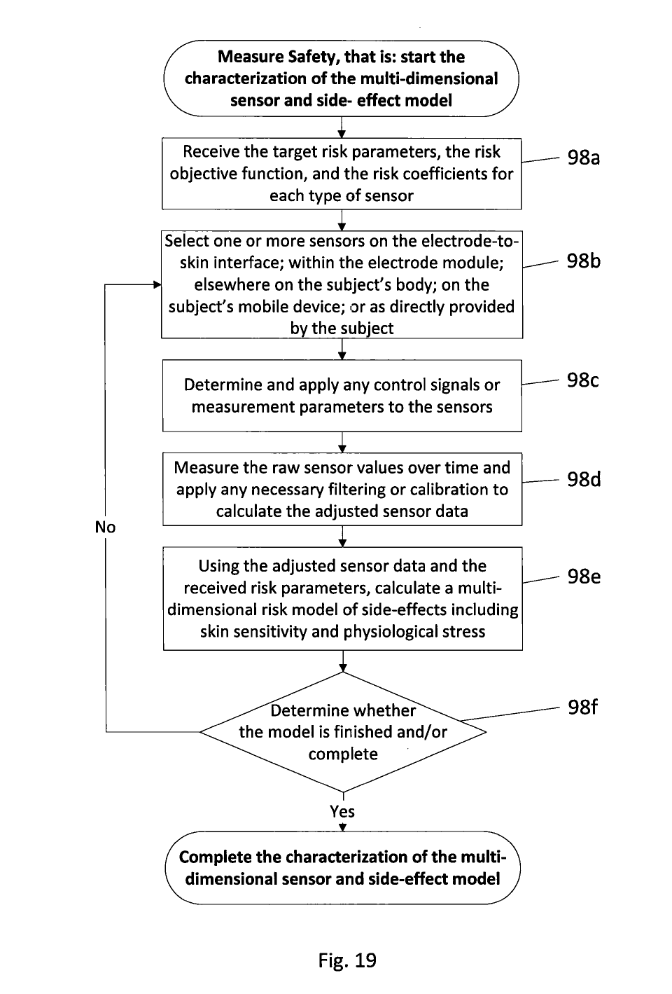

As illustrated by the flow diagram of FIG. 19, the control module 63 analyses inputs from the sensors, together with the input from the subject, and constructs a safety (mathematical) model of the side effects and risks of electrical stimulation applied between the electrode modules 46, 48, and an overall measure of safety and risk. In one example, a measure of safety and risk is a function of sensor data readings received from each of a plurality of the sensors (e.g. sensor A, sensor B and sensor C). That is:

risk=f(sensor_{A},sensor_{B},sensor_{C} . . . )

The function, f, may be a simple linear function comprising linear coefficients which operate on the received sensor data (which may also be normalised). The value of “risk” may be a simple scalar value determined from the function, f, and the sensor readings which can then be tested against the warning limit and abort limits (the abort limit being greater than the warning limit). If the risk value is greater than the warning limit but less than the abort limit, the control module 63 issues a warning, and if the risk value is greater than the abort limit, stimulation of the subject is aborted. The warning and abort limits can be determined from standard deviation limits for the sensor data readings from each of the sensors and the function, f.

More generally, with reference to FIG. 19, in step 98a the controller receives target risk parameters (which in the above example are the warning and abort limits), a risk objective function (which in the above example is the function, f) and risk coefficients (which in the above example are the linear coefficients used to construct f) for the respective sensors which are used by the risk objective function to weight the sensor data from the respective sensors. The target risk parameters define acceptable limits of the risk objective function. The risk objective function defines how to process the sensor data to determine whether it is indicative of a high or low level of safety risk. Risk coefficients are typically provided for each sensor. In some cases, risk parameters (e.g. warning and abort levels) may be provided for each sensor.

In a next step 98b, one or more of the sensors are selected and any control signals which need to be sent to the selected sensors (e.g. a signal which causes the LED of a colourimiter to direct light towards the skin interface) are determined and applied to the selected sensors in a next step 98c. In a next step 98d, raw sensor data from the selected sensors is detected, and may be filtered, scaled and/or calibrated to provide adjusted sensor data. The adjusted sensor data is then processed together with the relevant risk coefficient of the objective function. If the model is complete, the output of the risk objective function is calculated in a next step 98e. It may be that the sensor data from each of the sensors is also stored individually so that a location of a safety risk can be determined (e.g. by comparison of sensor data from each sensor individually with risk parameters provided for the respective sensors).

In a next step 98f, a determination is made as to whether the model is complete (e.g. whether data from all available sensors has been considered). If the model has not been completed, steps 98b to 98f are repeated. If all sensors have been considered, the model is considered to be complete and the risk objective function is calculated.

Referring back to FIG. 9, the risk objective function has been calculated, in a next step 230 the output of the risk objective function is compared to a safety limit (defined by one or more of the said risk parameters) to determine whether the level of stimulation being applied to the subject 40 is unsafe (e.g. the value of the risk objective function exceeds a limit set by a function risk parameter). If the risk objective function indicates that the stimulation being applied to the subject 40 is unsafe, the algorithm proceeds straight to step 142 and the stimulation is aborted. If the output of the risk objective function is within safe limits, it is then checked in a next step 232 against a warning limit (also defined by one or more of the said risk parameters) to determine whether the level of stimulation is undesirably high. If the output of the risk objective function exceeds the warning limit, the clinician and optionally the subject is warned in step 234 and the algorithm proceeds to step 147. In this case, the stimulation parameters may be adjusted manually by the clinician or according to a pre-determined algorithm determined by the clinician. Typically, this action would cause the stimulation amplitude to be reduced and optionally the stimulation session to be lengthened in order to achieve the same cumulative dosage. If the output of the risk objective function does not exceed the warning limit, the algorithm proceeds straight to step 147.

In some cases, if the output of the risk objective function exceeds the warning limit (but is within the safety limit), it may be that the control module 63 then compares each of the individual sensor readings to their associated individual risk parameters to determine which of the sensors are indicative of an undesirably high level of stimulation being applied to the subject. In this case (for the temperature, pH and colourimeter sensors), it may be that the localised sub-regions of the electrolyte sub-region where safety risks have been detected can be isolated and corrective action taken to reduce the level of stimulation being applied to that sub-region or additional electrolyte being dispensed to that sub-region.

As an alternative to a linear objective function, it may be that the risk objective function comprises a more complex neural network model derived from data from previous subjects (or from the same subject in previous stimulation sessions) together with their reported side effects.

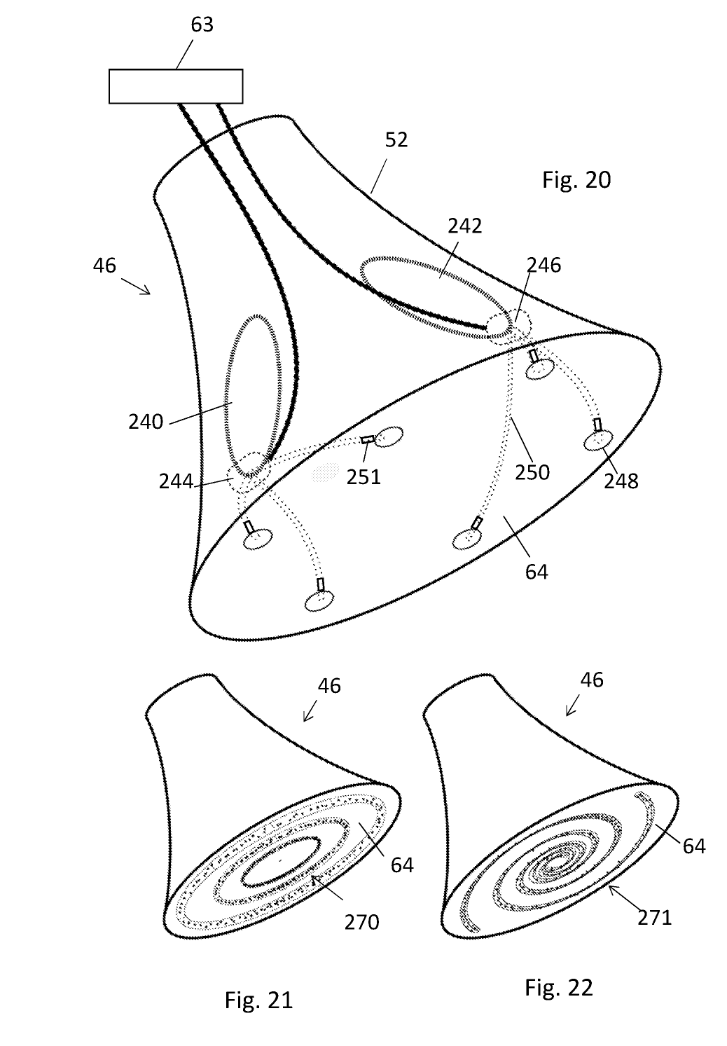

FIG. 20 is a perspective cut-away view of the electrode module 46 showing two electrolyte reservoirs 240, 242 contained within the electrode housing 52 and two piezo-electric electrolyte pumps 244, 246 configured to selectively dispense electrolyte from the electrolyte reservoirs 240, 242 to the electrolyte application region through a plurality of ducts 248 distributed across the surface 64 of the first end 54 of the electrode module 46 by way of electrolyte delivery lines 250 extending from the reservoirs 240, 242 to the ducts 248. The pumps 244, 246 are individually and selectively controlled by the control module 63 either wirelessly or by wire 252 to dispense electrolyte from the electrolyte reservoirs 240, 242 to the electrolyte application region 66. The pumps 244, 246 are also typically reversible so that they can suck electrolyte from the electrolyte application region back into the reservoirs 240, 242.

Electronically controlled valves 251 (e.g. MEMS microvalves, such as DMQ's silQflo microvalve) are provided in (or at an end of) each delivery line 250, the valves having open positions in which electrolyte can flow between one or more of the reservoirs and a respective duct 248, and closed positions in which electrolyte is blocked from flowing between the reservoirs and the respective duct 248. The control module 63 is in communication with the valves, and selectively controls whether each of the valves is in its open position or its closed position. By opening/closing valves 251, electrolyte can be selectively dispensed through individual ducts 248 so as to provide selective localised delivery or removal of electrolyte to one or more sub-regions of the electrolyte application region. This helps the control module 63 to provide the minimum quantity of electrolyte to the electrolyte application region necessary in order to provide good electrical contact between the electrodes and the skin interface, without causing excess mess on the head of the subject (and without causing safety issues or skin sensitivity). Although the valves 251 are shown in FIG. 20 adjacent to the ducts 258, it will be understood that they may be provided anywhere along (or at the ends of) the delivery lines 250.

As illustrated in FIG. 8A-8C discussed above, some or all of the electrodes of the electrode module 46 may be mounted on axial members 72, 72b, 72c extending from surface 64 of the electrode module 46. As also shown in FIGS. 8A-8C, it may be that the axial members 72, 72b, 72c are hollow such that they comprise internal axial channels 260, 260b, 260c which terminate in ducts 248.

As illustrated in FIG. 8D discussed above, in other embodiments it may be that the electrodes are mounted on the surface 64 of the electrode module 46 and on a plate 65 coupled and extending parallel to the said surface 64. In this case, axial channels 260d may be provided through the surface 64 and the plate 65 may comprise the ducts 248.

Typically the axial channels 260, 260b, 260c, 260d are provided in communication with respective electrolyte delivery lines 250 such that electrolyte from the electrolyte reservoirs 240, 242 can be delivered to the electrolyte application region 66 through the electrolyte delivery lines, axial channels 260, 260b, 260c, 260d and ducts 248.

The electrolyte reservoirs 240, 242 can be replenished between stimulation treatments. Alternatively, the reservoirs may be disposable, and new reservoirs 240, 242 may be installed at the beginning of each treatment session. As discussed above, a force actuator may be used to increase the electrode module-to-skin pressure in order to reduce the distance the electrolyte has to bridge. This is particularly important when hair is acting as a spring mechanism holding the electrode module 46 away from the skin.

FIGS. 21, 22 show alternative physical arrangements of electrolyte ducts. In these embodiments, the ducts are small permeable pipe, in concentric 270 or spiral arrangements 271 located on the surface 64 of the electrode module 46 and fed from the electrolyte reservoirs 240, 242.

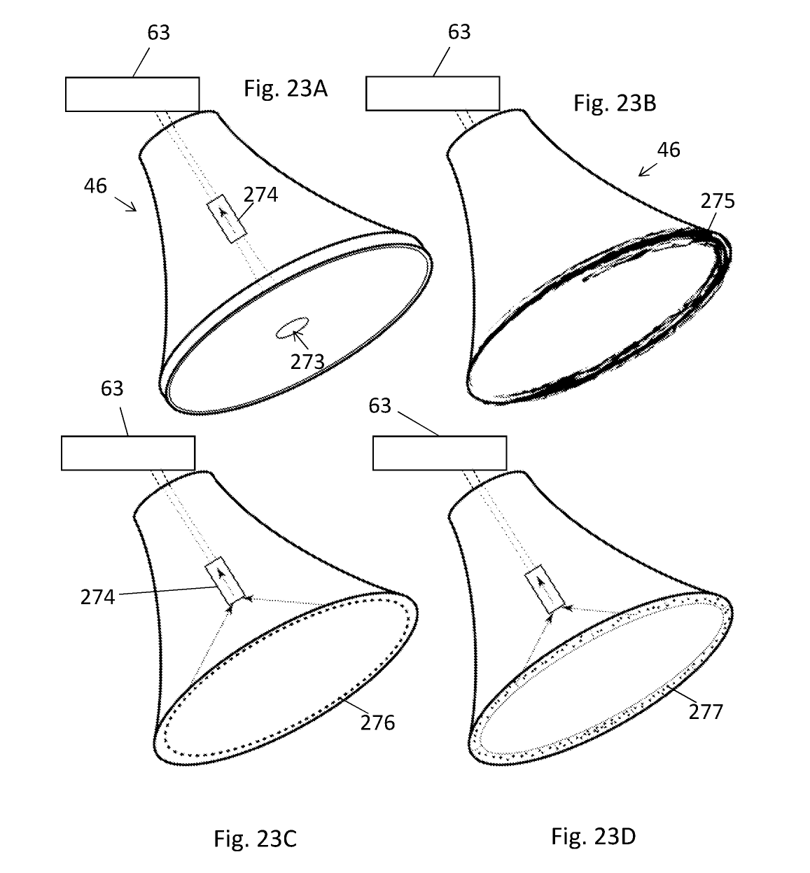

FIGS. 23A-23D illustrate apparatus for containing electrolyte in the electrolyte application region. As shown in FIG. 23A, a rubber rim 272 is provided around the perimeter of the end 54 of the electrode module 46 to contain electrolyte within the electrolyte application region 66. The rim 272 engages the scalp in use, thereby forming a seal which helps to prevent the electrolyte from flowing out of the electrolyte application region (and for example down the neck of the subject 40). FIG. 23A also provides an electrolyte removal duct 273 in communication with the electrolyte application region 66 and a vacuum (or negative pressure gradient) source 274 in communication with the electrolyte removal duct 273. The vacuum source is in electronic communication with the control module 63 which is configured to selectively activate the vacuum source 274 so as to remove electrolyte from the electrolyte application region 66 through the electrolyte removal duct 273. Typically the vacuum source 274 is configured to recycle electrolyte removed from the electrolyte application region into one or both of the reservoirs 240, 242 so that it can be re-used later.

As shown in FIG. 23B, an electrolyte absorber 275 (such as a paper washer) may be provided around the circumference of the end 54 of the electrode module 46 to help absorb excess electrolyte, thereby again helping to prevent the electrolyte from leaking out of the electrolyte application region and causing mess.

As shown in FIG. 23C, as an alternative to providing a single, central electrolyte removal duct 274 in communication with the vacuum source, a plurality of electrolyte removal ducts 276 may be provided, in this case distributed around the circumference of the first end 54 of the electrode module 46.

As shown in FIG. 23D, a porous rubber tube 277 may be provided around the circumference of the first end 54 of the electrode module 46, the porous rubber tube 277 forming a seal between the first end 54 and the skin interface 49 when the electrode module 46 is in use. Furthermore, the tube 277 is in communication with the vacuum source 274 such that the vacuum source can be selectively activated to remove excess electrolyte from the tube 277.

It will be understood that the vacuum source 274 and removal ducts 273, 276 could be omitted and the pumps 244, 246 could be used to apply a negative pressure gradient to suck electrolyte out of the electrolyte application region through ducts 248 instead.

This electrolyte containment apparatus helps to reduce electrolyte mess, and therefore makes treatment easier, more comfortable and quicker (including clean up). This also helps to reduce electrolyte drying, thereby helping to keep impedance constant throughout a stimulation session.

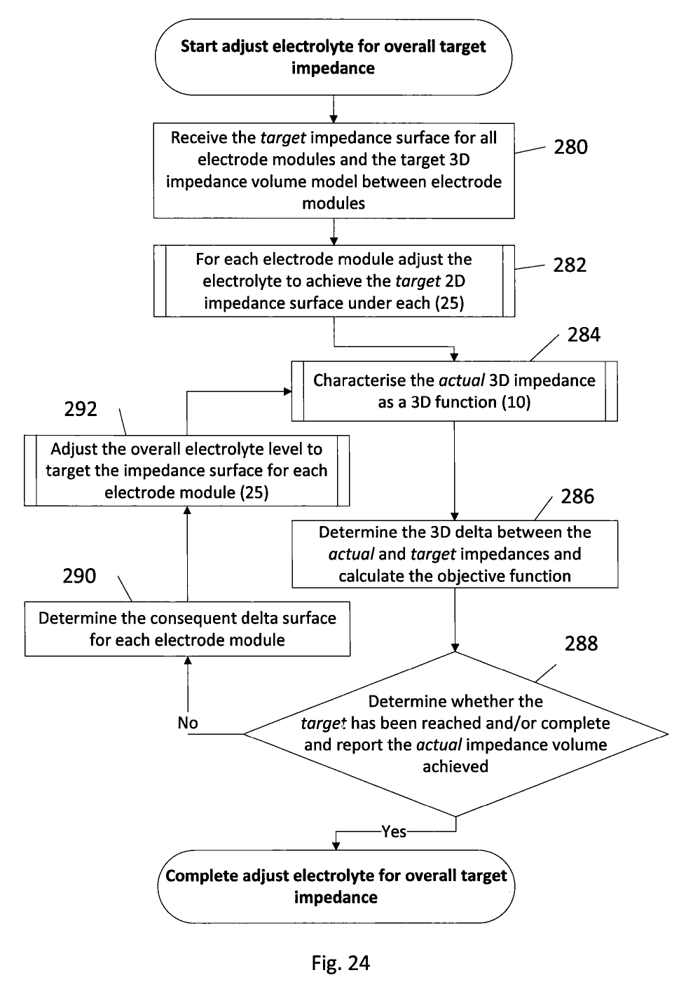

Referring back to the control algorithm of FIG. 9, at step 147 a check is made as to whether the full dosage has been applied to the target treatment region. If so, the stimulation session is complete. If not, two steps 278 and 279 are performed in parallel. In step 278, the electrolyte within the electrolyte application regions between the electrode modules 46, 48 and the skin interface 49 is adjusted using the algorithm of FIG. 24.

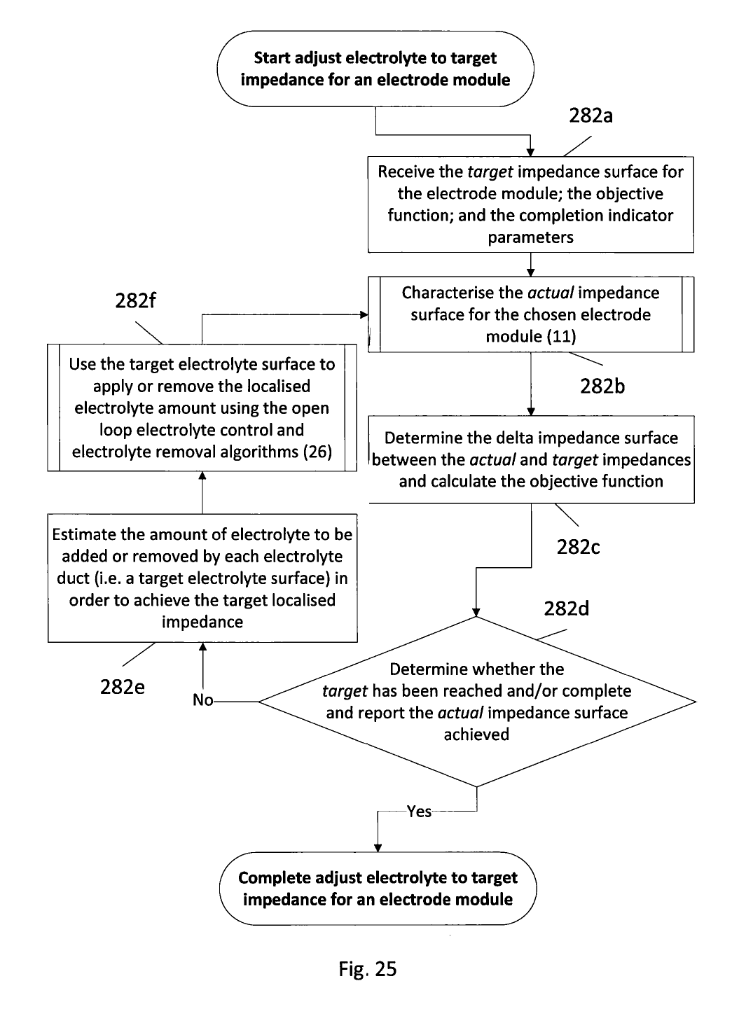

In a first step 280 a two dimensional target (mathematical) impedance surface is provided for each of the electrode modules 46, 48 (and others if provided) and a three dimensional target impedance volume model between the electrode modules 46, 48. In next step 282, for each electrode module 46, 48, the electrolyte provided in the electrolyte application region is adjusted in order to match the target two dimensional impedance surface using the algorithm described in FIG. 25.

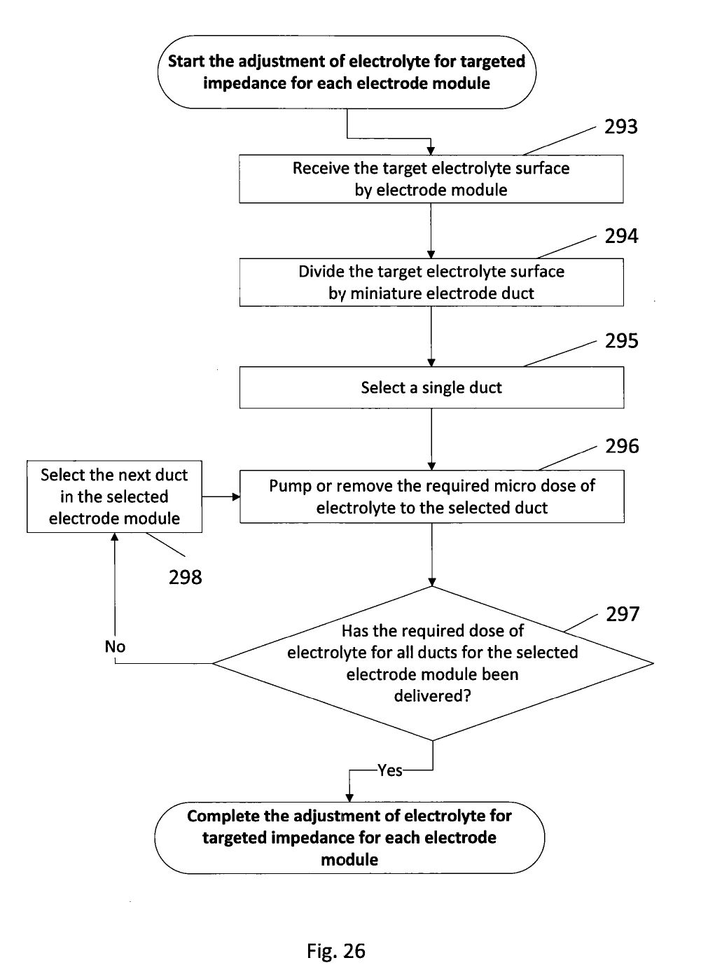

In a first step 282a, the target impedance mathematical surface (or volume) is received for the electrolyte application region of the first electrode module 46 together with an objective function (e.g. least squares error) and a set of completion values (e.g. target times or accuracies), which may be obtained from the clinician but more typically are set by the manufacturer. In a next step 282b, the impedance in the electrolyte application region 66 between the electrode module 46 and the skin interface is characterised using the algorithm of FIG. 11 to determine an impedance model of the electrolyte application region 66 as a function of position. In a next step 282c, the impedance model of the electrolyte application region 66 is compared to the target impedance (mathematical surface or volume) to determine a delta (mathematical surface or volume) indicative of the difference between them. In addition, the objective function is calculated from the delta. Next, in step 282d it is determined whether the objective function meets one or more accuracy criteria defined by the completion values (e.g. whether the least squares error objective function is less than a completion limit). If the objective function meets the completion values, the target is reached and the clinician notified. If not, an estimate is made in step 282e of the amount of electrolyte to be added to or removed from the electrolyte application region by way of each electrolyte duct 248 in the form of a target electrolyte mathematical surface. Next, the said estimated quantity of electrolyte is added to or removed from the electrolyte application region by way of each electrolyte duct 248 in step 282f using the algorithm of FIG. 26 and steps 282a to 282d are repeated.

As illustrated in FIG. 26, in a first step 293 the algorithm receives the estimate of electrolyte to be added to or removed from the electrolyte application region by way of each electrolyte duct 248 in the form of a target electrolyte mathematical surface. In a next step 294, the target electrolyte surface is divided by electrode duct 248 of the electrode module 46. In a next step 295, a single duct 248 of the electrode module 46 is selected. In a next step 296, the required quantity of electrolyte is pumped into or removed from the electrolyte application region through the selected duct 248 using the piezo-pump 244 in accordance with the portion of the target electrolyte surface comprising the selected duct 248 (and the valve 251 associated with that duct 248 is opened).

In a next step 297 a check is made as to whether the required quantity of electrolyte has been applied (or removed) for all ducts for the first electrode module 46. If not, the next duct is selected in step 298 and steps 296 and 297 are repeated for that duct. If the required quantity of electrolyte has been applied (or removed) for all ducts for the first electrode module 46, the algorithms of FIGS. 25 and 26 are repeated for electrode module 48 (and for any other modules provided).

Referring back to FIG. 24, following completion of step 282, in a next step 284, the three dimensional model of impedance (comprising the head impedance model and the impedance models of the electrolyte application regions) as a function of position is updated as discussed above with reference to FIGS. 10, 11. Next, in step 286, the three dimensional model of impedance as a function of position is compared to the target three dimensional volume impedance in order to determine a difference between them. An objective function (e.g. least squares error) is calculated from the difference and compared with an objective function completion value in step 288 to determine whether the three dimensional model of impedance as a function of position matches the target three dimensional volume impedance. If the two do not match, a delta is determined in step 290 for the electrolyte application regions of each electrode module which is determined from the difference between the three dimensional model of impedance and the target three dimensional volume impedance between electrode modules in respect of the electrolyte application region beneath that module. This delta is fed as an input to the algorithm of FIG. 25 (instead of the target two dimensional impedance surface initially used in the algorithm of FIG. 25) which is then repeated in step 292. Next, steps 284 to 292 are repeated until the three dimensional model of impedance as a function of position matches the target three dimensional volume impedance.

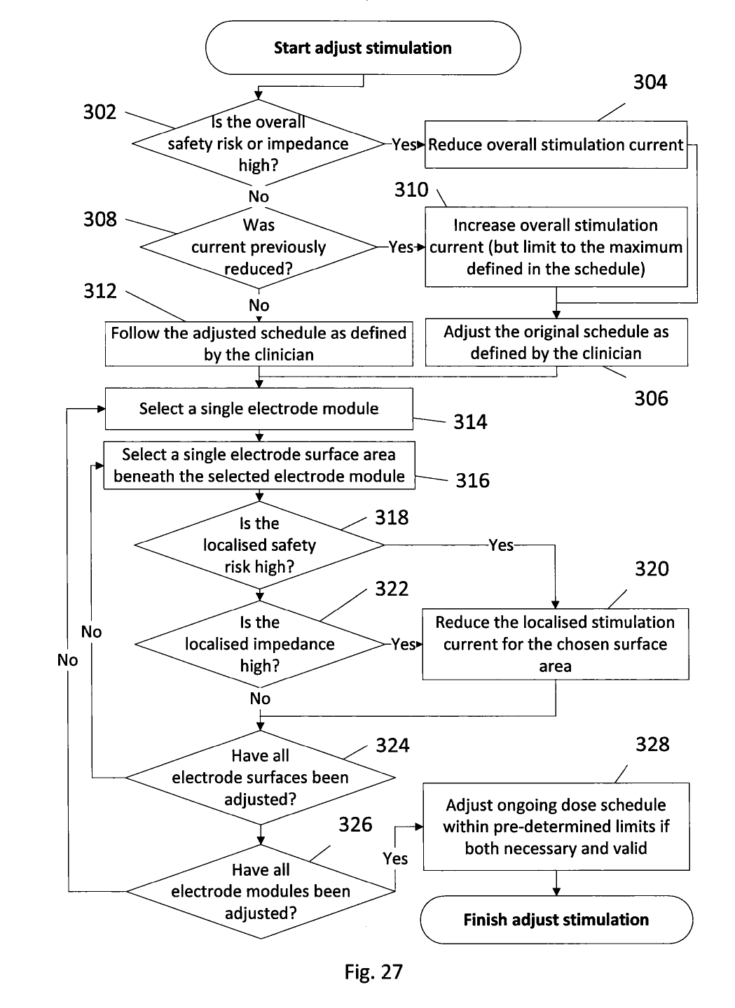

Referring back to FIG. 9, in step 279, the stimulation applied to the head is adjusted in accordance with the algorithm of FIG. 27. In a first step 302 the overall safety risk (i.e. the value of the risk objective function) determined using the algorithm of FIG. 19 and the impedances calculated using the algorithm of FIG. 10 are checked to determine whether either are above respective predetermined thresholds. If so, the overall stimulation current is reduced proportionally to the said overall safety risk or impedance level in step 304 and the original electrical stimulation schedule is adjusted in step 306. If neither the overall safety risk nor the impedances are above their respective predetermined thresholds, a check is made in step 308 as to whether the stimulation current was previously reduced in the current stimulation session. If so, the overall stimulation current applied to the head by the electrode modules 46, 48 is increased in step 310 taking into account the safety risk/impedances (but the increase is limited to the maximum defined in the schedule) and the original schedule defined by the clinician is adjusted in step 306. If the current was not previously reduced, the algorithm proceeds to step 312 whereby the stimulation follows the schedule as originally defined by the clinician or as otherwise adjusted by the clinician.

In next steps 314 to 326 it is determined as to whether the stimulation applied to the head should be locally adjusted as a result of a safety risk or (high) impedance in any localised sub-regions of the electrolyte application regions of the electrode modules 46, 48. In step 314, the first electrode module 46 is selected. Next, a localised sub-region (typically between a first electrode of the first electrode module 46 and the skin interface 49) of the electrolyte application region between the first electrode module 46 and the skin interface 49 is selected in step 316. Next, the localised safety risk determined in the algorithm of FIG. 19 at that localised sub-region (e.g. from a sensor such as a colourimeter in that localised sub-region) is checked in step 318 to determine whether it is above a threshold. If so, the localised stimulation current is reduced in step 320 for that localised sub-region, typically by reducing electrical signals applied between the first electrode and the second electrode module. If the localised safety risk for the localised sub-region is not above the threshold, a check is made in step 322 as to whether the localised impedance in the localised sub-region is above a threshold. If so, the localised stimulation current is reduced in step 320 for that localised sub-region as before. If the localised impedance in the localised sub-region is not above the threshold, a check is made in step 324 as to whether localised safety risk and localised impedances have been checked for all localised sub-regions of the electrolyte application region (typically each localised sub-region is between an electrode of the electrode module and the skin interface). If not, steps 316 to 324 are repeated for each localised sub-region. If the localised safety risk and localised impedances have been checked for all localised sub-regions of the electrolyte application region, the second electrode module 48 is selected in step 326 and steps 316 to 324 are repeated for the localised sub-regions of the second electrode module 48. When all electrode modules 46, 48 have been checked, the ongoing dose schedule is adjusted in step 328 within pre-determined limits set by the clinician if it is clinically necessary and valid to do so.

It will be understood that as part of the algorithm of FIG. 27, other parameters determined during step 96 can be tested against predetermined risk parameters, and the stimulation adjusted as a result. For example, current density in the localised sub-regions of the electrolyte application regions may be modelled during step 96 (or otherwise determined, e.g. derived from the impedance model) and tested in the algorithm of FIG. 27 to determine whether it exceeds a safety threshold at any of the localised sub-regions. In response to a determination that the current density in a localised sub-region exceeds a safety threshold, it may be that stimulation is aborted, or the current being supplied to that localised sub-region is reduced (or turned off). Additionally or alternatively, if the current density is lower than the safety threshold but exceeds a working threshold, it may be that electrolyte is selectively dispensed to that localised sub-region to reduce the current density in that sub-region (e.g. using the algorithm of FIG. 25).

The algorithm of FIG. 9 is then repeated until the stimulation session is complete. Accordingly, the various models are dynamically updated during a stimulation session.

Further modifications and variations may be made within the scope of the invention herein disclosed.

For example, current density in the electrolyte application regions may be modelled in addition to (e.g. in parallel with) the modelling of impedance in step 96; alternatively, current density in the electrolyte application regions may be modelled instead of the modelling of impedance in step 96 (the current density being indicative of impedance). Indeed, it is typically assumed that the bulk impedance of the electrolyte is constant, in which case one of the impedance and current density models can be determined from the other.

In another example, adjusting stimulation may involve reducing or increasing the amplitude of electrical stimulation signals applied between the electrodes of the first and second electrode modules 46, 48. Alternatively, adjusting stimulation may involve adjusting the shape of the electric field to better focus the dosage on the target treatment region (e.g. by selecting a particular sub-set of electrodes to deliver stimulation to the skin interface). Alternatively, adjusting stimulation may involve adjusting the shape of the electric field to reduce the current shunted across the skin interface between the modules 46, 48. Additionally or alternatively, the control module may be configured to reduce the physiological stress of the subject by adjusting a current distribution between electrodes of one both electrode modules. Additionally or alternatively, it may be that the control module 63 is configured to adjust the electrical signals applied to one or more or each of the electrodes of one or both of the electrode modules 46, 48 by adjusting any one or more of the following aspects of the electrical signals applied to one or more of the electrodes: the waveform; frequency content; and polarisation (e.g. by applying a DC offset) to thereby reduce physiological stress of the subject 40

In another example, the control module 63 may additionally or alternatively adjust the level of electrolyte in the electrolyte application regions between the electrode modules 46, 48 and the skin interface 49 responsive to one or more physiological stress indicators meeting one or more physiological stress criteria. For example, it may be that the control module 63 is configured, in step 234, to selectively dispense electrolyte to the electrolyte application region responsive to a determination that one or more of the said physiological stress indicators meet the first physiological stress criteria (and to adjust (e.g. reduce the amplitude of) electrical stimulation applied to the body portion by the electrodes responsive to a determination that one or more of the said physiological stress indicators meet the second physiological stress criteria as described). It may be that the controller is configured to reduce a physiological stress (e.g. skin sensitivity) specific to a localised sub-region of the electrolyte application region by individually (and typically selectively) dispensing electrolyte, or reducing electrical stimulation applied by one or more of the electrodes (e.g. by individually adjusting electrical signals applied to one or more electrodes), to a localised sub-region of the electrolyte application region responsive to a determination that one or more of the said physiological stress indicators specific to that sub-region meet one or more physiological stress criteria (e.g. are outside of an acceptable range).

Electrolyte may instead be dispensed to the electrolyte application regions by way of an open loop algorithm (such as that described in FIG. 26, with an open loop schedule of electrolyte dispensation being provided as the input instead of the estimates from the algorithm of FIG. 25).

Although the description above refers to the electrodes of the electrode modules 46, 48 being treated either individually or as a whole, it will be understood that one or more sub-sets of the electrodes may be grouped together (such that they can be treated as a single electrode) and signals applied across or between groups, or across or between a group and a single electrode or all of the electrodes of another module. Preferably, the hexagonal walls 132 between localised sub-regions illustrated in FIG. 12B are provided so as to help force stimulation signals through the head.

Although the above concepts are discussed in the context of the electrode modules 46, 48 which each have a plurality of electrodes, it will be understood that the concepts relating to controlling the quantity of electrolyte in the electrolyte application region, electrolyte containment, estimation of stimulation dosage, detection of current shunted across the skin interface and physiological stress indicator detection can each be applied to electrode modules having single electrodes. Similarly each of the aspects of the invention herein disclosed can be performed with one of the electrode modules having a plurality of electrodes spaced from each other as discussed, paired with a single pairing electrode (rather than a second electrode module having a plurality of electrodes as discussed), or paired with the electrodes of the second electrode module electrically coupled together such that they can be treated as a single pairing electrode (e.g. the same voltage being applied to each of the electrodes of the second electrode module).

The algorithms, apparatus and methods discussed herein, while described primarily for use in transcranial electrical stimulation, are also suitable, where applicable, for non-invasively applying electrical signals to or detecting electrical signals from other body portions of the subject.

Each of the features of the controller may implemented, for example, in software, hardware or a combination of software and hardware. The controller is typically distributed across a plurality of devices. It may be that at least part of the controller is provided in one or both of the electrode modules 46, 48. It may be that at least part of the controller is provided external to the electrode modules 46, 48 (e.g. in a laptop, desktop or tablet computer of the clinician or subject).

Various aspects of the invention are described by the numbered clauses below:
1. A method of non-invasively applying electrical stimulation to a body portion of a human subject by way of a skin interface, the method comprising: providing an electrode module having an end and a plurality of electrodes, the electrodes being spaced apart from each other; defining an electrolyte application region between the electrode module and the skin interface using the said end of the electrode module; electrically coupling the said electrodes to the skin interface by providing electrolyte in the said electrolyte application region; and individually adjusting electrical signals across or between each of the said electrodes and each of one or more pairing electrodes.
2. A method of non-invasively applying a dosage of electrical stimulation to a body portion of a human subject by way of a skin interface, the method comprising: providing an electrode module having an end and a plurality of electrodes, the electrodes being spaced apart from each other; defining an electrolyte application region between the electrode module and the skin interface using the said end of the electrode module; electrically coupling the said electrodes to the skin interface by providing electrolyte in the said electrolyte application region; applying a dosage of electrical stimulation to the body portion by applying electrical signals to each of the said electrodes; and individually adjusting electrical signals across or between each of the said electrodes and each of one or more pairing electrodes.
3. Electrode apparatus for non-invasively applying electrical stimulation to or detecting electrical signals from a body portion of a human subject by way of a skin interface, the electrode apparatus comprising:
   an electrode module having: an end for defining an electrolyte application region between the electrode module and the skin interface; and one or more electrodes which are electrically couplable or electrically coupled to the skin interface by way of an electrolyte in the said electrolyte application region;
   one or more electrolyte reservoirs containing electrolyte for electrically coupling the electrode(s) to the skin interface; and
   a controller configured to selectively dispense electrolyte from the electrolyte reservoir(s) to the electrolyte application region and/or to selectively remove electrolyte from the electrolyte application region.
4. The electrode apparatus according to clause 3 wherein the controller is configured to employ a closed-loop control system to control the selective dispensation of electrolyte from the electrolyte reservoir(s) to, and/or the selective removal of electrolyte from, the electrolyte application region.
5. The electrode apparatus according to clause 3 or clause 4 wherein the controller is provided with feedback, the controller being configured to selectively dispense electrolyte to, and/or selectively remove electrolyte from, the electrolyte application region responsive to the said feedback.
6. The electrode apparatus according to clause 5 wherein the controller is configured to selectively dispense electrolyte from the electrolyte reservoir(s) to the electrolyte application region responsive to a determination from the said feedback that an impedance or resistance of the electrolyte application region is outside of an acceptable range.
7. The electrode apparatus according to any one of clauses 3 to 6 wherein the controller is configured to dispense electrolyte from the electrolyte reservoir(s) to, and/or remove electrolyte from, the electrolyte application region by way of one or more electrolyte ducts extending to or through the said end of the electrode module.
8. The electrode apparatus according to clause 7 wherein the controller is configured to selectively dispense electrolyte from the electrolyte reservoir(s) to, and/or to selectively remove electrolyte from, the electrolyte application region through each of the said electrolyte ducts individually.
9. The electrode apparatus according to any one of clauses 3 to 8 wherein the controller is configured to selectively dispense electrolyte from the electrolyte reservoir(s) to, and or to selectively remove electrolyte from, each of a plurality of localised sub-regions within the electrolyte application region individually.
10. The electrode apparatus according to any one of clauses 3 to 9 wherein the electrode module comprises a plurality of electrodes spaced from each other across the said end of the electrode module.
11. The electrode apparatus according to clause 10 wherein each of a plurality of the electrodes of the electrode module are provided adjacent to a different electrolyte duct.
12. The electrolyte apparatus according to clause 5 or clause 6, or any one of clauses 7 to 11 as dependent on clause 5 or clause 6, wherein the controller is configured to selectively dispense electrolyte from the reservoir(s) to, and/or to selectively remove electrolyte from, one or more selected localised sub-regions of the electrolyte application region responsive to feedback specific to those sub-regions.
13. The electrode apparatus according to any of clauses 7 to 12 wherein the controller is configured to selectively dispense electrolyte into and/or selectively remove electrolyte from, the electrolyte application region by way of one or more electrolyte ducts, each of the electrolyte ducts being provided by a respective axial member extending to or through the said end of the electrode module.
14. The electrode apparatus according to clause 13 wherein at least one of the said one or more electrodes is mounted to a said axial member.
15. The electrode apparatus according to clause 14 wherein the said one or more electrodes comprises a plurality of electrodes, each of which is mounted to a said different one of the said axial members.
16. The electrode apparatus according to any one of clauses 7 to 15 further comprising one or more electrolyte flow directors in communication with the controller, the controller being configured to selectively dispense electrolyte from the electrolyte reservoir(s) to each of the electrolyte ducts individually by activating one or more of the electrolyte flow directors or a respective one of the electrolyte flow directors.
17. The electrolyte apparatus according to any one of clauses 3 to 16 wherein the one or more electrolyte reservoirs are re-fillable.
18. A method of non-invasively applying electrical stimulation to or detecting electrical signals from a body portion of a human subject by way of a skin interface, the method comprising: defining an electrolyte application region between an end of an electrode module and the skin interface, the said electrode module comprising one or more electrodes; providing one or more electrolyte reservoirs containing electrolyte for electrically coupling the electrode(s) to the skin interface; and electrically coupling the said one or more electrodes to the skin interface by selectively dispensing electrolyte from the electrolyte reservoir(s) to the electrolyte application region.
19. Electrode apparatus for non-invasively applying electrical stimulation to or detecting electrical signals from a body portion of a human subject by way of a skin interface, the electrode apparatus comprising:
   an electrode module having: an end for defining an electrolyte application region between the electrode module and the skin interface; one or more electrodes which are electrically couplable or electrically coupled to the skin interface by way of an electrolyte in the said electrolyte application region; and electrolyte containment apparatus configured to restrict leakage of electrolyte from the electrolyte application region.
20. The electrode apparatus according to clause 19 wherein the electrolyte containment apparatus comprises an electrolyte absorber provided on the said end of the electrode module.
21. The electrode apparatus according to clause 18 or clause 19 wherein the electrode module comprises a plurality of electrodes electrically couplable or electrically coupled to the skin interface by way of an electrolyte in the said electrolyte application region.
22. The electrode apparatus according to clause 21 wherein the electrolyte absorber at least partially surrounds at least some of the electrodes of the electrode module.
23. The electrode apparatus according to any one of clauses 19 to 22 wherein the electrolyte containment apparatus comprises a seal provided on the said end of the electrode module for restricting leakage of electrolyte from the electrolyte application region.
24. The electrode apparatus according to any one of clauses 19 to 23 wherein the electrolyte containment apparatus comprises a pressure gradient generator in fluid communication with the said end of the electrode module for restricting leakage of electrolyte from the electrolyte application region.
25. The electrode apparatus according to clause 24 wherein the pressure gradient generator is configured or configurable to apply a negative pressure gradient between the electrode module and the said end of the electrode module so as to restrict leakage of electrolyte from the electrolyte application region.
26. The electrode apparatus according to clause 24 or clause 25 wherein the electrolyte containment apparatus comprises a porous seal provided on the said end of the electrode module for restricting leakage of electrolyte from the electrolyte application region and a pressure gradient generator in communication with the said seal, the pressure gradient generator configured or configurable to apply a pressure gradient between one or more holes in the porous seal and the electrode module to thereby restrict leakage of electrolyte from the electrolyte application region.
27. The electrode apparatus according to any one of clauses 19 to 26 wherein the electrolyte containment apparatus comprises a plurality of walls provided at the end of the electrode module, the said walls defining localised sub-regions within the electrolyte application region and being configured to restrict electrolyte leakage from the said localised sub-regions when the said end of the electrode module is installed on the skin interface.
28. The electrode apparatus according to clause 27 wherein each of the localised sub-regions comprises one or more electrodes of the electrode module.
29. The electrode apparatus according to clause 28 wherein each of the localised sub-regions comprises one or more axial member on which one or more electrodes of the electrode module are mounted.
30. The electrode apparatus according to clause 28 or clause 29 wherein each of the localised sub-regions comprises one or more electrolyte duct through which electrolyte can be dispensed into the localised sub-region.
31. A method of non-invasively applying electrical stimulation to or detecting electrical signals from a body portion of a human subject by way of a skin interface, the method comprising: defining an electrolyte application region between an end of an electrode module and the skin interface, the said electrode module comprising one or more electrodes; electrically coupling the said electrode(s) to the skin interface by way of an electrolyte provided in the said electrolyte application region; and restricting leakage of electrolyte from the electrolyte application region.
32. Electrode apparatus for non-invasively applying electrical stimulation to a body portion of a human subject by way of a skin interface, the electrode apparatus comprising:
   an electrode module having: an end for defining an electrolyte application region between the electrode module and the skin interface; and one or more electrodes which are electrically couplable or electrically coupled to the skin interface by way of an electrolyte in the said electrolyte application region;
   a controller configured to apply electrical stimulation to the body portion by way of the one or more electrodes; and
   one or more sensors configured to measure one or more physiological stress indicators indicative of a physiological stress of the human subject,
   wherein the controller is further configured to: receive the said one or more measured stress indicators from the said sensors; determine whether one or more physiological stress criteria are met taking into account the measured physiological stress indicators; and provide an output responsive to a determination that the said physiological stress criteria are met.
33. The electrode apparatus according to clause 32 comprising first and second sensors, the first sensor being configured to measure a first said physiological stress indicator of the human subject and the second sensor being configured to measure a second said physiological stress indicator of the human subject different from the first physiological stress indicator.
34. The electrode apparatus according to clause 33 wherein the first said physiological stress indicator is an indicator of a first physiological stress of the subject and the second said physiological stress indicator is an indicator of a second physiological stress of the subject different from the first physiological stress.
35. The electrode apparatus according to any one of clauses 32 to 34 wherein the output provided responsive to the determination that the said physiological stress criteria are met comprises one or more signals for reducing the physiological stress of the human subject.
36. The electrode apparatus according to any one of clauses 32 to 35 wherein the output provided responsive to the determination that the said physiological stress criteria are met comprises one or more signals which adjust the electrical stimulation applied to the body portion by way of the one or more electrodes.
37. The electrode apparatus according to clause 36 wherein the electrical stimulation applied to the body portion is adjusted by reducing the amplitude of the electrical signals applied to one or more of the electrodes.
38. The electrode apparatus according to clause 36 or clause 37 wherein the electrode apparatus comprises a plurality of electrodes spaced apart from each other and wherein the controller is configured to adjust the electrical stimulation applied to the body portion by adjusting electrical signals applied to each of two or more of the electrodes.
39. The electrode apparatus according to any one of clauses 36 to 38 wherein the controller is configured to adjust the electrical stimulation applied to the body portion by adjusting any one or more of the following aspects of the electrical signals applied to one or more of the electrodes: the waveform; frequency content; and polarisation.
40. The electrode apparatus according to any one of clauses 32 to 39 wherein the output provided responsive to the determination that the said physiological stress criteria are met comprises a signal which causes electrical stimulation being applied to the body portion to be aborted.
41. The electrode apparatus according to any one of clauses 32 to 40 wherein the output provided responsive to the determination that the said physiological stress criteria are met comprises a signal which causes electrolyte to be selectively dispensed to the electrolyte application region.
42. The electrode apparatus according to any one of clauses 32 to 41 wherein the output provided responsive to a determination that first physiological stress criteria are met comprises a signal which causes electrolyte to be selectively dispensed to the electrolyte application region or a notification to be provided and the output provided responsive to a determination that second physiological stress criteria different from the first physiological stress criteria are met comprises a signal which causes the electrical stimulation applied to the body portion by the electrodes to be adjusted.
43. The electrode apparatus according to any one of clauses 32 to 42 wherein each of one or more of the said sensors are configured to measure a physiological stress indicator specific to a respective localised sub-region of the electrolyte application region, wherein the controller is configured to determine whether one or more localised physiological stress criteria are met taking into account the measured physiological stress indicator and to provide an output specific to the said localised sub-region responsive to a determination that said one or more localised physiological stress criteria specific to that sub-region are met.
44. The electrode apparatus according to any one of clauses 32 to 43 wherein the said one or more sensors comprise one or more sensors configured to measure a physiological stress indicator which comprises a physiological parameter of the body portion.
45. The electrode apparatus according to any one of clauses 32 to 44 wherein the one or more sensors comprise one or more sensors configured to measure one or more physiological stress indicators indicative of a skin sensitivity of the human subject.
46. The electrode apparatus according to any one of clauses 43 to 45 wherein the said one or more sensors comprise one or more colourimeters configured to measure a parameter indicative of a colour of the body portion.
47. The electrode apparatus of clause 46 wherein the said one or more colourimeters are configured to measure a parameter indicative of a red or infrared colour of the body portion.
48. The electrode apparatus according to any one of clauses 32 to 47 wherein the one or more sensors comprise one or more sensors configured to measure one or more physiological stress indicators indicative of a pre-ictal state of the subject.
49. The electrode apparatus according to any one of clauses 32 to 48 wherein the one or more sensors comprise one or more movement sensors.
50. The electrode apparatus according to any one of clauses 32 to 49 wherein the one or more sensors comprise one or more or each of the electrodes of the electrode module configured to operate in an electroencephalography (EEG) mode.
51. A method of non-invasively applying electrical stimulation to a body portion of a human subject by way of a skin interface, the method comprising: defining an electrolyte application region between an end of an electrode module and the skin interface, the electrode module comprising one or more electrodes; electrically coupling the one or more electrodes to the skin interface by way of an electrolyte provided in the said electrolyte application region; applying electrical stimulation to the body portion by way of the electrode(s); measuring one or more physiological stress indicators indicative of a physiological stress of the human subject; determining whether one or more physiological stress criteria are met taking into account the measured physiological stress indicators; and providing an output responsive to a determination that the said physiological stress criteria are met.
52. Electrode apparatus for non-invasively applying electrical stimulation to a body portion of a human subject by way of a skin interface, the electrode apparatus comprising:
   a first electrode module having: an end for defining a first electrolyte application region between the first electrode module and the skin interface, the first electrode module comprising one or more electrodes which are electrically couplable or electrically coupled to the skin interface by way of an electrolyte in the said first electrolyte application region;
   a second electrode module having: an end for defining a second electrolyte application region between the second electrode module and the skin interface, the second electrode module comprising one or more electrodes which are electrically couplable or electrically coupled to the skin interface by way of an electrolyte in the said second electrolyte application region;
   one or more shunt measurement conductors; and
   a controller configured to: determine one or more electrical parameters between one or more electrodes of the first electrode module and one or more of the shunt measurement conductors; and determine a current shunted across the skin interface between the first and second electrode modules taking into account the said one or more determined electrical parameters.
53. The electrode apparatus according to clause 52 wherein one or more or each of the shunt measurement conductors are provided on or adjacent to the said end of the first electrode module.
54. The electrode apparatus according to clause 52 or clause 53 wherein one or more or each of the shunt measurement conductors are provided between the electrode(s) of the first electrode module and an edge of the said end of the first electrode module.
55. The electrode apparatus according to any one of clauses 52 to 54 wherein the controller is configured to: apply one or more electrical test signals between one or more electrodes of the first electrode module and one or more of the shunt measurement conductors; determine one or more electrical parameters across or between the said electrodes of the first electrode module and the said shunt measurement conductors responsive to the said test signal; and determine the said current shunted across the skin interface between the first and second electrode modules taking into account the said one or more determined electrical parameters.
56. The electrode apparatus according to any one of clauses 52 to 55 wherein the controller is configured to: apply electrical test signals between the said electrodes of the first electrode module and the said electrodes of the second electrode module; determine one or more electrical parameters between the said one or more electrodes of the first electrode module and the one or more electrodes of the second electrode module; and determine the said current shunted across the skin interface between the first and second electrode modules further taking into account the said one or more electrical parameters determined across or between the said one or more electrodes of the first electrode module and the one or more electrodes of the second electrode module.
57. The electrode apparatus according to any one of clauses 52 to 56 wherein the one or more shunt measurement conductors comprises a plurality of shunt measurement conductors spaced apart from each other.
58. The electrode apparatus according to any one of clauses 52 to 57 wherein the one or more shunt measurement conductors comprises one or more first shunt measurement conductors and one or more second shunt measurement conductors, the first shunt measurement conductors being positioned closer to the electrodes of the first electrode module than the second shunt measurement conductors are to the electrodes of the first electrode module.
59. The electrode apparatus according to clause 58 wherein the controller is configured to determine the said current shunted across the skin interface of the said body portion by measuring an electrical parameter between or across one or more of the first shunt measurement conductors and one or more of the second shunt measurement conductors.
60. The electrode apparatus according to any one of clauses 52 to 59 wherein the controller is configured to estimate a dosage of electrical stimulation impinging on a or the target treatment region of the body portion in response to electrical signals applied between the electrode(s) of the first and second electrode modules taking into account the determined current shunted across the skin interface.
61. The electrode apparatus according to any one of clauses 52 to 60 wherein the second electrode module comprises one or more of the said shunt measurement conductor(s).
62. The electrode apparatus according to clause 61 wherein the controller is configured to determine one or more electrical parameters between one or more electrodes of the second electrode module and the shunt measurement conductors of the second electrode module; and determine the said current shunted across the skin interface between the first and second electrode modules taking into account the said one or more determined electrical parameters between one or more electrodes of the second electrode module and the shunt measurement conductors of the second electrode module.
63. The electrode apparatus according to any one of clauses 52 to 62 wherein the first and second electrode modules each comprise one or more shunt measurement conductor(s), and wherein the controller is configured to determine the said current shunted across the skin interface of the said body portion taking into account one or more electrical parameters determined across or between one or more shunt measurement conductors of the first electrode module and one or more shunt measurement conductors of the second electrode module.
64. The electrode apparatus according to any one of clauses 52 to 63 wherein the controller is configured to adjust electrical signals applied to one or more electrodes of one or both of the first and second electrode modules to thereby reduce the current shunted across the skin interface between the first and second electrode modules.
65. A method of non-invasively applying electrical stimulation to a body portion of a human subject by way of a skin interface, the method comprising: defining a first electrolyte application region between an end of a first electrode module and the skin interface, the first electrode module comprising one or more electrodes; electrically coupling the said one or more electrodes of the first electrode module to the skin interface by providing an electrolyte in the said first electrolyte application region; defining a second electrolyte application region between an end of a second electrode module and the skin interface, the second electrode module comprising one or more electrodes; electrically coupling the said one or more electrodes of the second electrode module to the skin interface by providing an electrolyte in the said first electrolyte application region; providing one or more shunt measurement conductors; measuring one or more electrical parameters between one or more electrodes of the first electrode module and one or more of the shunt measurement conductors; and determining a current shunted across the skin interface between the first and second electrode modules taking into account the said one or more measured electrical parameters.
66. Data processing apparatus comprising a computer processor, the data processing apparatus being configured to: receive geometry data representing a geometry of a human body portion comprising a target treatment region internal to the body portion; receive impedance data indicative of one or more impedances or resistances of the said body portion; determine electric field data representing an electrical field through the body portion, which is responsive to an electrical stimulation applied to the body portion by way of a skin interface of the body portion, taking into account the geometry data and the impedance data; and determine a dosage of electrical stimulation impinging on the target treatment region from the electric field data.
67. Data processing apparatus according to clause 66 wherein the geometry data comprises a mathematical model and/or image of the body portion.
68. Data processing apparatus according to clause 66 or clause 67 wherein the geometry data represents a three dimensional geometry of the human body portion.
69. Data processing apparatus according to any one of clauses 66 to 68 wherein the geometry data represents a geometry of both an external portion of the body portion and an internal portion of the body portion.
70. Data processing apparatus according to any one of clauses 66 to 69 wherein the impedance data comprises data indicative of an impedance or resistance of a first type of human tissue external to the body portion and data indicative of an impedance or resistance of a second type of human tissue internal to the body portion.
71. Data processing apparatus according to any one of clauses 66 to 70 further configured to: determine electric field data representing an electrical field applied through the body portion responsive to an electrical stimulation applied to the body portion by way of a skin interface of the body portion taking into account the geometry data and the impedance data by: using the said geometry data and the impedance data to mathematically model an electric field through the body portion responsive to the said electrical stimulation.
72. Data processing apparatus according to any one of clauses 66 to 71 further configured to determine a dosage of electrical stimulation impinging on the target treatment region from the determined electric field data using predetermined data indicative of the position of the target treatment region within the body portion.
73. Data processing apparatus according to any one of clauses 66 to 72 configured to: provide electrical signals between an electrode and a pairing electrode to thereby apply electrical stimulation to the body portion by way of the skin interface; determine electric field data representing the electrical field applied through the body portion responsive to the said electrical stimulation applied to the body portion taking into account the geometry data and the impedance data; and determine a dosage of electrical stimulation impinging on the target treatment region from the electric field data.
74. Data processing apparatus according to clause 73 further configured to adjust the electrical signals applied between the electrode and the said pairing electrode to thereby adjust the electrical stimulation applied to the body portion responsive to the determined dosage of electrical stimulation impinging on the target treatment region.
75. Data processing apparatus according to clause 73 or clause 74 further configured to receive an estimate of an electrical current shunted across the skin interface between the said electrode and the pairing electrode, the data processing apparatus being further configured to determine the dosage of electrical stimulation impinging on the target treatment region from the said determined electric field data taking into account the said estimate of the said electrical current shunted across the skin interface.
76. Data processing apparatus according to any one of clauses 66 to 75 configured to determine a dosage of electrical stimulation impinging on the target treatment region by volume integration of the determined electric field through the target treatment region.
77. A method of estimating a dosage of electrical stimulation impinging on a target treatment region internal to a human body portion, the method comprising: providing geometry data representing a geometry of the human body portion comprising the target treatment region internal to the body portion; providing impedance data indicative of one or more impedances or resistances of the said body portion; determining an electrical field applied through the body portion, which is responsive to an electrical stimulation applied to the body portion by way of a skin interface of the body portion, taking into account the geometry data and the impedance data; and determining a dosage of electrical stimulation impinging on the target treatment region from the determined electric field data.
78. The method according to clause 77 wherein the geometry data comprises a mathematical model and/or image of the body portion.
79. The method according to clause 77 or clause 78 wherein the geometry data represents a three dimensional geometry of the human body portion.
80. The method according to any one of clauses 77 to 79 wherein the impedance data is indicative of impedances or resistances of two or more different types of human tissue of the said body portion.
81. The method according to clause 80 wherein the impedance data comprises data indicative of an impedance or resistance of a first type of human tissue external to the body portion and data indicative of an impedance or resistance of a second type of human tissue internal to the body portion.
82. The method according to any one of clauses 77 to 81 further comprising: determining an electrical field applied through the body portion responsive to an electrical stimulation applied to the body portion taking into account the geometry data and the impedance data by using the said geometry data and the impedance data to mathematically model the electric field applied through the body portion responsive to the said electrical stimulation.
83. The method according to any one of clauses 77 to 82 further comprising determining a dosage of electrical stimulation impinging on the target treatment region from the determined electric field data using predetermined data indicative of the position of the target treatment region within the body portion.
84. The method according to any one of clauses 77 to 83 further comprising: providing electrical signals between an electrode and a pairing electrode to thereby apply electrical stimulation to the body portion by way of the skin interface; determining electric field data representing the electrical field applied through the body portion responsive to the electrical stimulation applied to the body portion by the electrodes taking into account the geometry data and the impedance data; and determining a dosage of electrical stimulation impinging on the target treatment region from the electric field data.
85. The method according to clause 84 further comprising adjusting the electrical signals applied between the electrode and the pairing electrode to thereby adjust the electrical stimulation applied to the body portion responsive to the determined dosage of electrical stimulation impinging on the target treatment region.
86. The method according to clause 84 or clause 85 further comprising receiving an estimate of an electrical current shunted across the skin interface between the said electrode and the pairing electrode; and determining the dosage of electrical stimulation impinging on the target treatment region from the said determined electric field data taking into account the said estimate of the electrical current shunted across the skin interface.
87. The method according to any one of clauses 77 to 86 further comprising determining a dosage of electrical stimulation impinging on the target treatment region by volume integration of the electric field data relating to the target treatment region.
88. An electrode module for non-invasively applying electrical stimulation to a body portion of a human subject by way of a skin interface, the electrode module having: an end for defining an electrolyte application region between the electrode module and the skin interface; one or more electrodes which are electrically couplable or electrically coupled to the skin interface by way of an electrolyte in the said electrolyte application region; and one or more shunt measurement conductors provided between the said electrode(s) and an edge of the said end of the electrode module.

1. Electrode apparatus for non-invasively applying electrical stimulation to a body portion of a human subject by way of a skin interface, the electrode apparatus comprising:
an electrode module having: an end for defining an electrolyte application region between the electrode module and the skin interface; and a plurality of electrodes which are electrically couplable or electrically coupled to the skin interface by way of an electrolyte in the said electrolyte application region, the electrodes being spaced apart from each other; and
a controller in communication with the electrodes, the controller being configured to individually adjust electrical signals across or between each of the said electrodes and each of one or more pairing electrodes.

2. The electrode apparatus according to claim 1 wherein the controller is configured to determine a spatial distribution of current flow within the said electrolyte application region by: individually adjusting electrical signals across or between each of two or more of the said electrodes of the electrode module and each of one or more pairing electrodes; determining one or more respective electrical parameters which are responsive to the adjusted electrical signals; and determining the spatial distribution of current flow within the said electrolyte application region from the said determined electrical parameters.

3. The electrode apparatus according to claim 2 wherein the controller is configured to determine the said spatial distribution of current flow within the said electrolyte application region by: determining a parameter indicative of at least the magnitude of the current flowing within each of a plurality of localised sub-regions of the electrolyte application region from the said determined electrical parameters.

4. The electrode apparatus according to claim 2 wherein the controller is configured to determine the spatial distribution of electrical current within the electrolyte application region by individually adjusting electrical signals already being applied between one or more of the electrodes of the electrode module and the said one or more pairing electrodes.

5. The electrode apparatus according to claim 1 wherein the controller is configured to determine an impedance or resistance of a localised sub-region of the electrolyte application region by: individually adjusting electrical signals across or between a said electrode of electrode module and each of one or more pairing electrodes; determining one or more electrical parameters which are responsive to the adjusted electrical signals; and determining the impedance or resistance of the localised sub-region from the said determined electrical parameters.

6. The electrode apparatus according to claim 1 wherein the controller is configured to determine the impedance or resistance of each of a plurality of localised sub-regions of the electrolyte application region by: individually adjusting electrical signals across or between each of the said plurality of the said electrodes of the electrode module and each of one or more pairing electrodes; determining one or more respective electrical parameters which are responsive to the adjusted electrical signals; and determining the impedance or resistance of each of the localised sub-regions from the respective determined electrical parameters.

7. The electrode apparatus according to claim 1 wherein the controller is configured to: receive geometry data representing a geometry of the body portion, the body portion comprising a target treatment region internal to the body portion; receive impedance data indicative of one or more impedances or resistances of the said body portion; determine electric field data representing an electric field through the body portion, which is responsive to an electrical stimulation applied by electrical signals between one or more of the electrodes of the electrode module and one or more pairing electrodes, taking into account the said geometry data and the impedance data; and determine a dosage of electrical stimulation impinging on the target treatment region from the electric field data.

8. The electrode apparatus according to claim 7 wherein at least a portion of the geometry data is determined by electrical impedance tomography or electrical impedance spectroscopy of the body portion using the electrodes of the electrode module.

9. The electrode apparatus according to claim 7 wherein the impedance data comprises data indicative of an impedance or resistance of a first type of human tissue external to the body portion and data indicative of an impedance or resistance of a second type of human tissue internal to the body portion.

10. The electrode apparatus according to claim 7 wherein the controller is configured to: determine electric field data indicative of an electrical field through the body portion responsive to an electrical stimulation applied to the body portion by the electrodes using the geometry data and the impedance data by: using the said geometry data and the impedance data to mathematically model the electric field applied through the body portion as a function of position responsive to the said electrical stimulation.

11. The electrode apparatus according to claim 1 wherein the controller is configured to determine an impedance model indicative of the impedance or resistance of the body portion as a function of position by: individually adjusting electrical signals across or between each of the said plurality of the said electrodes of the electrode module and each of one or more respective pairing electrodes; determining one or more electrical parameters indicative of one or more respective impedances or resistances of the body portion; and determining the impedance model from the said determined parameters.

12. The electrode apparatus according to claim 11 wherein the controller is configured to: determine electric field data representing an electric field through the body portion, which is responsive to an electrical stimulation applied by electrical signals between one or more of the electrodes of the electrode module and one or more pairing electrodes, taking into account the impedance model; and determine from the electric field data a dosage of electrical stimulation impinging on a target treatment region internal to the body portion responsive to an electrical stimulation applied to the skin interface by the said electrodes.

13. The electrode apparatus according to claim 11 wherein the controller is configured to: provide an initial impedance model; and adjust the initial impedance model by individually applying electrical signals between each of the said electrodes and each of one or more pairing electrodes, in each case measuring a voltage across and/or a current flowing between each of the said electrodes of the electrode module and the said one or more pairing electrodes, and adjusting the impedance model in accordance with the said measured voltages across and/or currents flowing between each of the said electrodes of the electrode module and the said one or more pairing electrodes.

14. The electrode apparatus according to claim 13 wherein the controller is configured to adjust the impedance model by: individually applying electrical signals between each of the said electrodes and each of one or more pairing electrodes, the electrical signals comprising electrical signals of different frequencies; determining a frequency response to the said electrical signals of a voltage across and/or a current flowing between each of the said electrodes of the electrode module and the said one or more pairing electrodes; and adjusting the impedance model in accordance with the said determined voltages across and/or currents flowing between each of the said electrodes of the electrode module and the said one or more pairing electrodes.

15. The electrode apparatus according to claim 7 wherein the controller is configured to determine a dosage of electrical stimulation impinging on the target treatment region using predetermined data indicative of the position of the target treatment region within the body portion.

16. The electrode apparatus according to claim 1 wherein the electrode module is a first electrode module and the electrode apparatus further comprises: a second electrode module having: an end for defining a second electrolyte application region between the second electrode module and the skin interface, the second electrode module comprising one or more electrodes which are electrically couplable or electrically coupled to the skin interface by way of an electrolyte in the said second electrolyte application region; and one or more shunt measurement conductors in communication with the controller, wherein the controller is configured to: measure one or more electrical parameters between one or more electrodes of the first electrode module and one or more of the shunt measurement conductors; and determine a current shunted across the skin interface between the first and second electrode modules taking into account the said one or more measured electrical parameters.

17. The electrode apparatus according to claim 16 wherein the controller is configured to: apply one or more electrical test signals between one or more electrodes of the first electrode module and one or more of the shunt measurement conductors; measure one or more electrical parameters across or between the said electrodes of the first electrode module and the said shunt measurement conductors responsive to the said test signal; and determine the said current shunted across the skin interface between the first and second electrode modules taking into account the said one or more measured electrical parameters.

18. The electrode apparatus according to claim 16 wherein the controller is configured to: apply one or more electrical test signals between the said electrodes of the first electrode module and the said electrodes of the second electrode module; measure one or more electrical parameters between the said one or more electrodes of the first electrode module and the one or more electrodes of the second electrode module; and determine the said current shunted across the skin interface between the first and second electrode modules further taking into account the said one or more electrical parameters measured across or between the said one or more electrodes of the first electrode module and the one or more electrodes of the second electrode module.

19. The electrode apparatus according to claim 16 wherein the one or more shunt measurement conductors comprises one or more first shunt measurement conductors and one or more second shunt measurement conductors, the first shunt measurement conductors being positioned closer to the electrodes of the first electrode module than the second shunt measurement conductors are to the electrodes of the first electrode module.

20. The electrode apparatus according to claim 19 wherein the controller is configured to determine the said current shunted across the skin interface of the said body portion by measuring an electrical parameter across or between one or more of the first shunt measurement conductors and one or more of the second shunt measurement conductors.

21. The electrode apparatus according to claim 16 wherein the controller is configured to estimate a dosage of electrical stimulation impinging on a or the target treatment region of the body portion in response to electrical signals applied between the electrode(s) of the first and second electrode modules taking into account the determined current shunted across the skin interface.

22. The electrode apparatus according to claim 16 wherein the second electrode module comprises one or more of the said shunt measurement conductor(s).

23. The electrode apparatus according to claim 1 wherein the controller is configured to selectively dispense electrolyte into the said electrolyte application region, and/or to selectively remove electrolyte from, the electrolyte application region.

24. The electrode apparatus according to claim 23 wherein the controller is configured to employ a closed-loop control system to control the selective dispensation the electrolyte reservoir(s) to and/or removal of electrolyte from the electrolyte application region.

25. The electrode apparatus according to claim 23 wherein the controller is provided with feedback, the controller being configured to selectively dispense electrolyte to, and/or remove electrolyte from, the electrolyte application region responsive to the said feedback.

26. The electrode apparatus according to claim 25 wherein the controller is configured to dispense electrolyte from the electrolyte reservoir(s) to the electrolyte application region responsive to a determination from the said feedback that an impedance or resistance or current density of the electrolyte application region is outside of an acceptable range.

27. The electrode apparatus according to claim 23 wherein the controller is configured to dispense electrolyte from the electrolyte reservoir(s) to, and/or remove electrolyte from, the electrolyte application region by way of one or more electrolyte ducts provided at, and/or extending through, the said end of the electrode module.

28. The electrode apparatus according to claim 23 wherein the controller is configured to dispense electrolyte to, and/or to remove electrolyte from, the electrolyte application region by way of a plurality of electrolyte ducts which are spaced apart from each other across the said end of the electrode apparatus.

29. The electrode apparatus according to claim 28 wherein the controller is configured to selectively dispense electrolyte from the electrolyte reservoir(s) to, and/or to selectively remove electrolyte from, the electrolyte application region through each of the said electrolyte ducts individually.

30. The electrode apparatus according to claim 23 wherein the controller is configured to selectively dispense electrolyte into, and/or selectively remove electrolyte from, the electrolyte application region by way of one or more electrolyte ducts, each of the electrolyte ducts being provided by a respective axial member extending to or through the said end of the electrode module.

31. The electrode apparatus according to claim 30 wherein the controller is configured to selectively dispense electrolyte into, and/or selectively remove electrolyte from, each of a plurality of localised sub-regions of the electrolyte application region individually by way of an electrolyte duct of a said axial member provided in or adjacent to the said localised sub-region.

32. The electrode apparatus according to claim 29 further comprising one or more electrolyte flow directors in communication with the controller, the controller being configured to selectively dispense electrolyte to, and/or selectively remove electrolyte from, the electrolyte application region by activating one or more of the electrolyte flow directors or a respective electrolyte flow director.

33. The electrode apparatus according to claim 1 wherein the controller is configured to adjust electrical signals applied across or between one or more selected electrodes of the electrode module and one or more pairing electrodes responsive to a determination that the impedance or resistance or a current density between one or more of the said electrodes of the electrode module and the skin interface exceeds a predetermined threshold.

34. The electrode apparatus according to claim 1 further comprising electrolyte containment apparatus for restricting leakage of electrolyte from the electrolyte application region.

35. The electrode apparatus according to claim 34 wherein the electrolyte containment apparatus comprises an electrolyte absorber provided on the said end of the electrode module.

36. The electrode apparatus according to claim 34 wherein the electrolyte containment apparatus comprises a seal provided on the said end of the electrode module for restricting leakage of electrolyte from the electrolyte application region.

37. The electrode apparatus according to claim 34 wherein the electrolyte containment apparatus comprises a pressure gradient generator in communication with the said end of the electrode module for restricting leakage of electrolyte from the electrolyte application region.

38. The electrode apparatus according to claim 37 wherein the pressure gradient generator is configured or configurable to apply a negative pressure gradient between an internal portion of the electrode module and the said end of the electrode module so as to restrict leakage of electrolyte from the electrolyte application region.

39. The electrode apparatus according to claim 1 further comprising one or more sensors configured to measure one or more physiological stress indicators indicative of a physiological stress of the human subject, wherein the controller is configured to: receive the said one or more measured stress indicators from the said sensors; determine whether one or more physiological stress criteria are met taking into account the measured physiological stress indicators; and provide an output responsive to a determination that the said physiological stress criteria are met.

40. The electrode apparatus according to claim 39 wherein the output provided responsive to the determination that the said physiological stress criteria are met comprises one or more signals which adjust the electrical stimulation applied to the body portion by way of the said electrodes.

41. The electrode apparatus according to claim 39 wherein the output provided responsive to the determination that the said physiological stress criteria are met comprises a signal which causes electrolyte to be selectively dispensed to the electrolyte application region.

42. The electrode apparatus according to claim 1 wherein each of one or more of the said sensors are configured to measure a physiological stress indicator specific to a respective localised sub-region of the electrolyte application region, wherein the controller is configured to determine whether one or more localised physiological stress criteria are met taking into account the measured physiological stress indicator and to provide an output specific to the said localised sub-region responsive to a determination that said one or more localised physiological stress criteria specific to that sub-region are met.

43. A non-transitory computer-readable medium computer readable carrier storing computer program code for individually adjusting electrical signals across or between each of the said electrodes of the electrode apparatus of claim 1 and each of one or more pairing electrodes.

44. An electrode module for non-invasively applying electrical stimulation to a body portion of a human subject by way of a skin interface, the electrode module having: an end for defining an electrolyte application region between the electrode module and the skin interface; and a plurality of electrodes which are electrically couplable or electrically coupled to the skin interface by way of an electrolyte in the said electrolyte application region, the electrodes being spaced apart from each other, wherein the said electrodes are configured so that electrical signals to each of the said electrodes can be adjusted individually.

45. A method of non-invasively applying electrical stimulation to a body portion of a human subject by way of a skin interface, the method comprising: providing an electrode module having an end and a plurality of electrodes, the electrodes being spaced apart from each other; defining an electrolyte application region between the electrode module and the skin interface using the said end of the electrode module; electrically coupling the said electrodes to the skin interface by providing electrolyte in the said electrolyte application region; and individually adjusting electrical signals across or between each of the said electrodes and each of one or more pairing electrodes.

46. A method of non-invasively applying a dosage of electrical stimulation to a body portion of a human subject by way of a skin interface, the method comprising: providing an electrode module having an end and a plurality of electrodes, the electrodes being spaced apart from each other; defining an electrolyte application region between the electrode module and the skin interface using the said end of the electrode module; electrically coupling the said electrodes to the skin interface by providing electrolyte in the said electrolyte application region; applying a dosage of electrical stimulation to the body portion by applying electrical signals to each of the said electrodes; and individually adjusting electrical signals across or between each of the said electrodes and each of one or more pairing electrodes.
